(19) United States
(12) Patent Application Publication
(10) Pub. No.: US 2024/0052436 A1
(43) Pub. Date: Feb. 15, 2024

(54) CRISPR-BASED SARS-COV-2 DETECTION

(71) Applicant: SHERLOCK BIOSCIENCES, Brighton, MA (US)

(72) Inventors: Xiang Li, Malden, MA (US); Mary Katherine Wilson, Burlington, MA (US); Christine Marie Coticchia, Brookline, MA (US); Brendan John Manning, Brighton, MA (US); William Jeremy Blake, Winchester, MA (US); Elizabeth Mae Selleck Fiore, Brighton, MA (US)

(21) Appl. No.: 18/009,832

(22) PCT Filed: Jun. 11, 2021
(86) PCT No.: PCT/US21/36925

§ 371 Date: Dec. 12, 2022

Related U.S. Application Data
(60) Provisional application No. 63/185,268, filed on May 6, 2021.
(60) Provisional application No. 63/139,268, filed on Jan. 19, 2021.
(60) Provisional application No. 63/068,817, filed on Aug. 21, 2020.
(60) Provisional application No. 63/056,523, filed on Jul. 24, 2020.
(60) Provisional application No. 63/054,214, filed on Jul. 20, 2020.
(60) Provisional application No. 63/038,715, filed on Jun. 12, 2020.

(51) Int. Cl. C12Q 1/70

(52) U.S. Cl. CPC C12Q 1/701; C12Q 2600/16

(57) ABSTRACT

The present disclosure provides methods and compositions for the detection of SARS-CoV-2 by CRISPR/Cas collateral RNAse activity.

### CROSS REFERENCE TO RELATED APPLICATIONS

This application claims priority to each of U.S. Provisional Patent Application Nos. 63/038,715 filed Jun. 12, 2020; 63/054,214 filed Jul. 20, 2020; 63/056,523 filed Jul. 24, 2020; 63/068,817 filed Aug. 21, 2020; 63/139,268 filed Jan. 19, 2021; 63/185,268 filed May 6, 2021 the entire contents of each of which are hereby incorporated by reference.

### BACKGROUND

SARS-CoV-2, first identified in humans in December 2019, causes coronavirus disease 2019 (COVID-19), and was declared a global pandemic by the World Health Organization on Mar. 11, 2020. The hallmark of productive public health management of any and all outbreaks is the ability to test for individuals to identify their infection status. There is a present desperate need for improved detection and diagnostic technologies.

### SUMMARY

The present disclosure provides compositions and methods for the detection and diagnosis of SARS-CoV-2.

### BRIEF DESCRIPTION OF DRAWING

FIG. 1 presents open reading frames of SARS-CoV-2; SARS-CoV; and MERS-CoV.

FIG. 2 provides an exemplary workflow for detection of SARS-CoV-2 described herein.

FIG. 3: Plotted means of three replicates (n=3) of sample detection of 30 cp/μL of target 1 (orf1ab) and 6 cp/μL of target 2 (N), along with positive (5000 cp/μL) and negative controls (0 cp/μL).

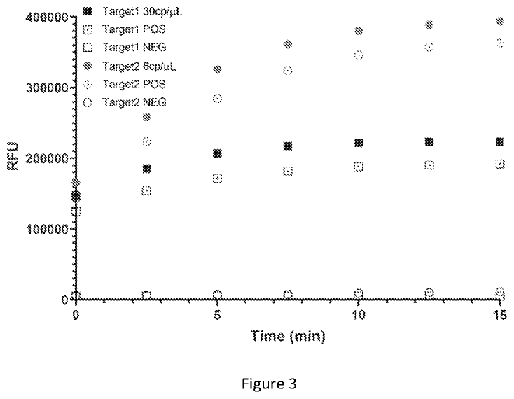

FIG. 4: Signal-to-background ratio linear scale plot at 15 minutes (T15/T0) of individual replicates for target 1 (orf1ab) and target 2 (N).

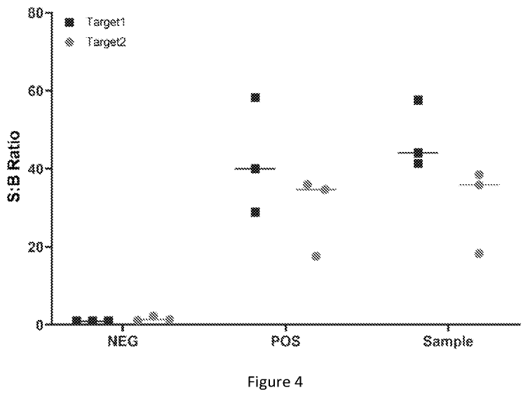

FIG. 5: Signal-to-background ratio linear scale plot at 10 minutes (T10/T0) of individual replicates for target 1 (orf1ab) and target 2 (N).

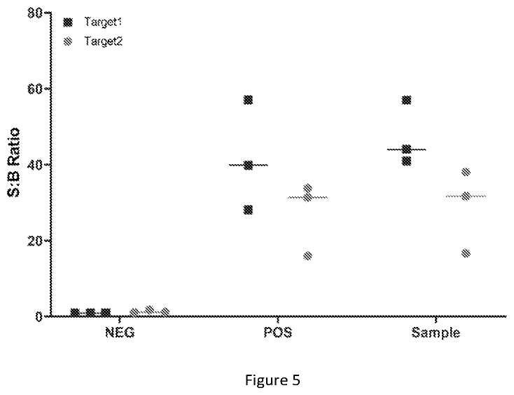

FIG. 6: Signal-to-background ratio log scale plot at 15 minutes (T15/T0) of individual replicates for target 1 (orf1ab) and target 2 (N).

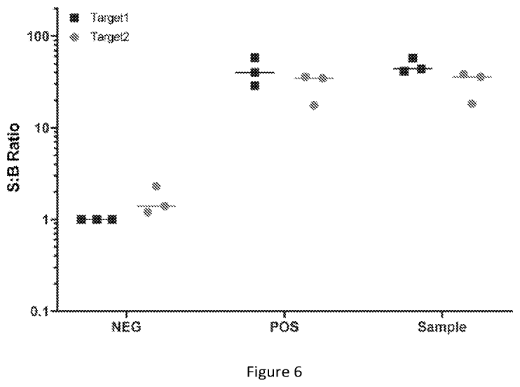

FIG. 7: Signal-to-background ratio log scale plot at 10 minutes (T10/T0) of individual replicates for target 1 (orf1ab) and target 2 (N).

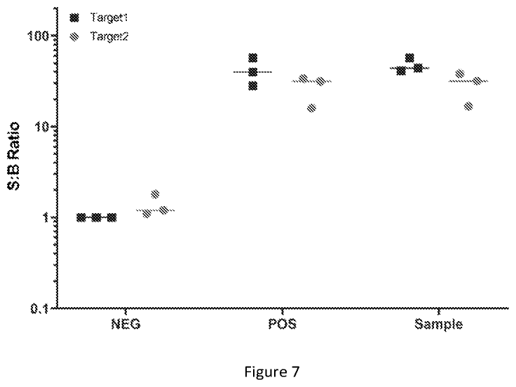

FIG. 8: Plotted means of twenty replicates (n=20) of sample detection of 45 cp/μL of target 1 (orf1ab) and 9 cp/μL of target 2 (N), along with positive (5000 cp/μL) and negative controls (0 cp/μL).

FIG. 9: Signal-to-background ratio log scale plot at 15 minutes (T15/T0) of individual replicates target 1 (orf1ab) and target 2 (N). Results are considered to be negative if the S:B ratio for the sample is <5.0.

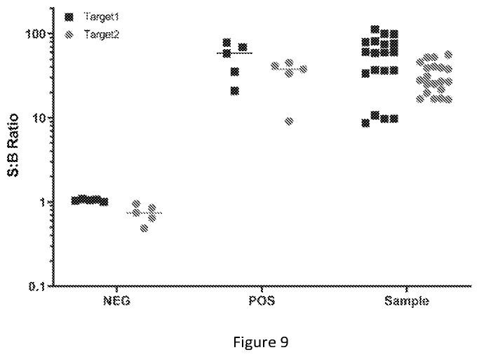

FIG. 10: Signal-to-background ratio log scale plot at 10 minutes (T10/T0) of individual replicates for target 1 (orf1ab) and target 2 (N). Results are considered to be negative if the S:B ratio for the sample is <5.0.

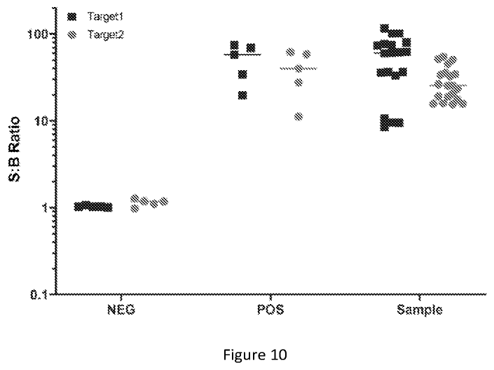

FIG. 11 shows an exemplary workflow of a combined workflow as described herein.

FIG. 12 shows results of detecting SARS-CoV-2 (N and Orf1ab (“O”) using a combined “automated” workflow as described herein.

FIG. 13 shows a comparison of RFUs of the combined workflow relative to a standard workflow.

FIG. 14 shows a comparison of SARS-CoV-2 containing saliva samples extracted using methods described herein and assayed using the combined workflow (“new workflow”).

FIG. 15 further demonstrates the sensitivity of detection using SARS-CoV-2 containing saliva samples extracted using methods described herein and assayed using the combined workflow.

FIG. 16 demonstrates the ability of the duplexed system (DARTSv1) to detect both SARS-CoV-2 and RP simultaneously.

FIG. 17 shows evaluation of the limit of detection of DARTSv1.

FIG. 18 demonstrates the ability of the duplexed system (DARTSv2) to detect both SARS-CoV-2 and RP simultaneously.

FIG. 19 shows evaluation of the limit of detection of DARTSv2.

FIG. 20 shows concordance of DARTSv2 with PCR SARS-CoV-2 detection results on unextracted NP swab clinical samples.

FIG. 21 shows concordance of DARTSv2 with PCR SARS-CoV-2 detection results on extracted clinical samples.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Designed: As used herein, the term “designed” refers to an agent (i) whose structure is or was selected by the hand of man; (ii) that is produced by a process requiring the hand of man; and/or (iii) that is distinct from natural substances and other known agents.

Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gel: As used herein, the term “gel” refers to viscoelastic materials whose rheological properties distinguish them from solutions, solids, etc. In some embodiments, a composition is considered to be a gel if its storage modulus (G′) is larger than its modulus (G″). In some embodiments, a composition is considered to be a gel if there are chemical or physical cross-linked networks in solution, which is distinguished from entangled molecules in viscous solution.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

In vitro: The term “in vitro” as used herein refers to events that occur in an artificial environment, e.g., in a test tube or reaction vessel, in cell culture, etc., rather than within a multi-cellular organism.

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g, clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, and/or condition displays one or more symptoms of a disease, disorder, and/or condition and/or has been diagnosed with the disease, disorder, or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

### Detailed Description of Certain Embodiments

### SARS-CoV-2

In some embodiments, the present disclosure provides compositions and methods for detection and/or diagnosis of SARS-CoV-2. SARS-CoV-2 is the causative agent of COVID-19. According to the United States Centers for Disease Control (“CDC”), early symptoms of COVID-19 often include one or more of: fever/chills, cough, shortness of breath or difficulty breathing, fatigue, muscle or body aches, headache, new loss of taste or smell, sore throat, congestion or runny nose, nausea or vomiting, and/or diarrhea. More serious symptoms often include, for example, trouble breathing, persistent pain or pressure in the chest, new confusion, inability to wake or stay awake, and/or bluish lips or face. Alternatively or additionally, COVID-19 patients may display low blood oxygenation (e.g., below 98%), and/or one or more symptoms or features of acute respiratory distress syndrome (ARDS) and/or pneumonia.

Reports suggest that individuals over age 60, and/or those with underlying immune conditions, may have particularly high risk of developing COVID-19 after exposure to and infection with SARS-CoV-2.

SARS-CoV-2 is a virus in the coronavirus family. Members of the coronavirus family are lipid membrane viruses with a positive sense single stranded RNA genome.

SARS-CoV-2 genomes have been sequenced from multiple human samples; such sequences are generally available, for example, through publication and/or deposit in publically-accessible databases. See NCBI Reference Sequence: NC_045512.2; Severe acute respiratory syndrome coronavirus 2 data hub (www.ncbi.nlm.nih.gov/labs/virus/vssi/#/virus?SeqType_s=Nucleotide&VirusLineage_ss=SARS-CoV-2,%20taxid:2697049); www.ncbi.nlm.nih.gov/genbank/sars-cov-2-seqs/#reference-genome.

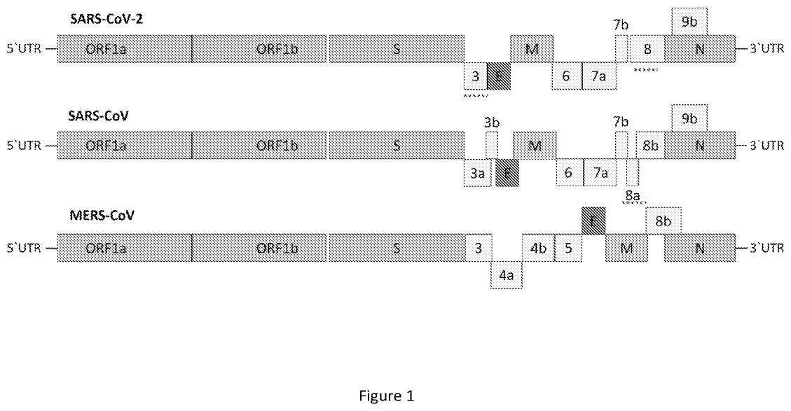

FIG. 1 presents a representation of the SARS-CoV genomes and the open reading frames in includes. As can be seen, the genome encodes concated protein that is processed by the virally encoded protease.

### CRISPR Cas Collateral Activity

Recently, certain CRISPR/Cas enzymes have been identified that have an ability to non-specifically cleave collateral nucleic acid(s) when activated by binding to a target site recognized by the guide RNA with which they are complexed. Representative examples of Cas12, Cas13, and Cas14 enzymes have been shown to have such collateral cleavage activity. See, for example, Swarts and Jinek Mol Cell. 2019 Feb. 7; 73(3):589-600.e4; Harrington L. B. et al. Science. 2018; 362: 839-842; Li S. Y. et al. Cell Res. 2018; 28: 491-493; Chen J. S. et al., Science. 2018; 360: 436-439; Abudayyeh O. O. et al., Science. 2016; 353aaf5573; East-Seletsky A et al., Nature. 2016; 538: 270-273; Gootenberg J S et al.; Science 2017; 356:438-442; Myhrvold C, et al., Science 2018; 360:444-448; Gootenberg J S et al., Science 2018; 360:439-444. Some CRISPR/Cas enzyme collateral cleavage activity digests or cleaves single strand nucleic acids. Some CRISPR/Cas enzyme collateral cleavage activity digests or cleaves double stranded nucleic acids. Some CRISPR/Cas enzyme collateral cleavage activity digests or cleaves RNA. Some CRISPR/Cas enzyme collateral cleavage activity digests or cleaves DNA. Some CRISPR/Cas enzyme collateral cleavage activity digests or cleaves both RNA and DNA.

This collateral activity has been harnessed to develop detection (e.g., diagnostic) technologies that achieve detection of nucleic acids containing the relevant target site (or its complement) in biological and/or environmental sample(s). See, for example Gootenberg J S et al.; Science 2017; 356:438-442; WO2019/011022; U.S. Pat. Nos. 10,494,664B2; 10,337,051B2; 10,266,887; sherlock.bio/better-faster-affordable-diagnostic-testing.

The present disclosure provides particularly effective technology for detecting SARS-CoV-2 in biological and/or environmental samples, including by providing examples of effective such detection. For example, the present disclosure exemplifies detection of SARS-CoV-2 in nucleic acid isolated from nasopharyngeal swabs, utilizing certain Cas13 enzyme(s).

The present disclosure describes particular reagents—e.g., target sites, guide RNA sequences, amplification and/or signal generation technologies, and combinations thereof that together achieve important and surprising sensitivity and/or specificity for SARS-CoV-2 detection. The present disclosure also describes, for example, samples, formats, and various conditions (e.g., temperature, time, concentration of components etc) surprisingly effective in detecting SARS-CoV-2.

The present disclosure also identifies the source of certain problems and/or provides key insights that permit such achievement.

Provided Detection Technologies

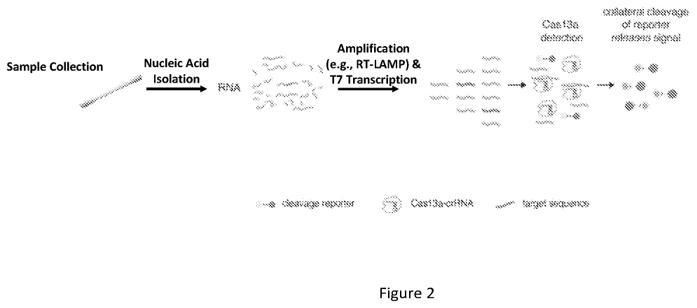

FIG. 2 provides a workflow overview of a detection assay as exemplified herein. The assay depicted in FIG. 2 includes steps of:
(i) sample collection
(ii) target isolation/amplification
(iii) CRISPR/Cas collateral activity

The present disclosure provides insights and/or technologies relevant to each of these steps. In some embodiments, multiple steps described herein can be performed simultaneously. In some embodiments, one or more steps described herein can be performed in a single vessel, e.g., a one-pot reaction. In some embodiments, amplification and CRISPR/Cas collateral activity can occur in a single vessel.

The particular isolation technology used in isolation/amplification step is, in some embodiments, any sample processing that results in nucleic acid. One of skill in the art is aware of many sample processing techniques that result in stable nucleic acid isolation.

The particular target isolation/amplification technology depicted in FIG. 2 involves loop-mediated isothermal amplification (LAMP). In some embodiments, the amplification step comprises reverse transcription LAMP (RT-LAMP). Those skilled in the art will be aware that certain reported CRISPR/Cas Collateral Activity Detection methodologies (see, e.g., utilize alternative amplification technologies such as, for example, Nucleic Acid Sequence Based Amplification (NASBA); Strand Displacement Amplification; Recombinase Polymerase Amplification (RPA); Rolling Circle Amplification (RCA)). In some embodiments, one or more of such alternative amplification technologies may be employed in the practice of the present invention (e.g., together with other aspects and/or features described herein). However, the present disclosure identifies that, in certain embodiments, LAMP may be preferable at least because it provides increased speed and specificity and operates at a single constant temperature.

Those skilled in the art will be aware that certain software packages have been specifically developed for use with LAMP technologies, including to predict sequences of primers that are expected to be useful for any given target nucleic acid. Among other things, the present disclosure identifies the source of a problem with such predictions, and surprisingly finds particular sequences that demonstrate unexpected utility relative to others generated by such predictions.

In some embodiments, the amplification step comprises primers that comprise a promoter sequence. In some embodiments, primers comprise a RNA polymerase promoter sequence. In some embodiments, a RNA polymerase promoter sequence allows for transcription of DNA to RNA prior to the CRISPR/Cas enzyme detection. In some embodiments, a RNA polymerase promoter comprises pol I, pol II, pol III, T7, T3, SP6, U6, H1, retroviral Rous sarcoma virus (RSV) LTR promoter, the cytomegalovirus (CMV) promoter, the SV40 promoter, the dihydrofolate reductase promoter, the .beta.-actin promoter, the phosphoglycerol kinase (PGK) promoter, and the EF1.alpha. promoter.

The particular CRIPR/Cas collateral activity assay depicted in FIG. 2 utilizes a Cas13 enzyme. Those skilled in the art will be aware of numerous Cas13 enzymes useful for the assays described herein. Further, those skilled in the art will be aware of numerous methods, algorithms, and software for guide polynucleotide design. See e.g., sgRNA Designer (Broad) CRISPR Targeted Gene Designer (Horizon Discovery), https://en.wikipedia.org/wiki/CRISPR/Cas_Tools.

Sections below discuss in more detail various features and/or embodiments of certain aspects of provided technologies.

CRISPR/Cas Enzymes

In some embodiments, methods and compositions of the present disclosure utilize CRISPR/Cas enzymes. In some embodiments, methods and compositions of the present disclosure utilize Type V, or Type Type VI CRISPR/Cas enzymes. In some embodiments, methods and compositions of the present disclosure utilize Cas12, Cas13, and/or Cas14 CRISPR/Cas enzymes. In some embodiments, methods and compositions of the present disclosure utilize CRISPR/Cas enzymes described in WO2016/166340; WO2016/205711; WO/2016/205749; WO2016/205764; WO2017/070605; WO/2017/106657. In some embodiments, methods and compositions of the present disclosure utilize Cas13a CRISPR/Cas enzymes. In some embodiments, methods and compositions of the present disclosure utilize LwaCas13a CRISPR/Cas enzymes.

In some embodiments, methods and compositions of the present disclosure utilize thermostable CRISPR/Cas enzymes. In some embodiments, methods and compositions of the present disclosure utilize thermostable CRISPR/Cas enzymes encoded by sequences listed in table 1.

Lengthy table referenced here
US20240052436A1-20240215-T00001
Please refer to the end of the specification for access instructions.

The present disclosure teaches that, in some embodiments, it will be particularly desirable or useful to utilize a thermostable Cas enzyme. In some embodiments, a useful thermostable Cas protein is a Cas12 or Cas13 homolog (e.g., ortholog). In some embodiments, a useful thermostable Cas protein is a Cas enzyme comprising an amino acid sequence having 80%, 85%, 90%, 99% or 100% sequence identity to any one of SEQ ID Nos. 1-71 or 530-741.

Alternatively or additionally, in some embodiments, a useful thermostable Cas protein performs (e.g., its collateral cleavage activity functions sufficiently) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In many embodiments, useful thermostable Cas protein performs (e.g., its collateral cleavage activity functions sufficiently) at temperatures above about 60° C.

In some embodiments, a useful thermostable Cas protein performs (e.g., its collateral cleavage activity functions sufficiently) within a temperature range at which nucleic acid extension and/or amplification reaction(s) are performed; those skilled in the art are well familiar with various such reactions and the temperature ranges at which they are performed, In some embodiments, such a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 75° C.

The present disclosure furthermore teaches that a thermostable Cas enzyme as described herein may be particularly useful when and/or may permit multiple reaction steps to be performed in a single reaction/vessel (e.g., for “one pot” reactions). Thus, in some embodiments, use of a thermostable Cas may reduce or eliminate certain processing and/or transfer steps. In some embodiments, all reaction steps beyond nucleic acid isolation may be performed in a single vessel (e.g., in a “one pot” format).

Guide Polynucleotides

In some embodiments, the present disclosure provides guide polynucleotides. that recognize and bind a target nucleic acid of interest. In some embodiments, a guide polynucleotide is a guide RNA (gRNA, sgRNA). In some embodiments guide polynucleotides of the present disclosure comprise a crRNA. In some embodiments a crRNA is complementary to a target nucleic acid of interest.

Those skilled in the art will be aware of numerous methods to design and identify guide polynucleotides for a target nucleic acid of interest. Those skilled in the art will be aware of numerous algorithms and software useful to design guide polynucleotides for a target nucleic acid of interest.

The present disclosure used available algorithms to design guides based on available SARS-CoV-2 sequences. The present disclosures describes tests to empirically identify which, if any, of those guide polynucleotides suggested by existing algorithms were useful in the presently described methods and compositions. As described further in the Examples, only those guide RNAs specifically identified and empirically tested as described in this disclosure were useful for the detection of SARS-CoV-2 in the presently described CRISPR based detection assay.

In some embodiments, a guide polynucleotides has 60%, 70%, 80%, 90%, 95%, 90% sequence identity to a sequence listed in Table 23 In some embodiments, a guide polynucleotide comprises a crRNA disclosed in Table 17. In some embodiments, a crRNA used in a guide polynucleotide has 60%, 70%, 80%, 90%, 95%, 90% sequence identity to a crRNA listed in Table 17

LAMP

As noted above, among other things, the present disclosure provides certain LAMP technologies, and/or components thereof, whose particular usefulness and/or effectiveness is documented herein. In some embodiments, amplification is performed as described in WO2000/028082; WO2001/034790; WO2001/077317; or WO2002/024902.

One of skill in the art will be aware of numerous method to design primers useful in LAMP. The present disclosures describes tests to empirically identify which, if any, of those LAMP primers suggested by existing algorithms were useful in the presently described methods and compositions. As described further in the Examples, only those LAMP primers specifically identified and empirically tested as described in this disclosure were useful for the detection of SARS-CoV-2 in the presently described CRISPR based detection assay.

In some embodiments, a LAMP primer has 60%, 70%, 80%, 90%, 95%, 90% sequence identity to a sequence listed in Table 20. has 60%, 70%, 80%, 90%, 95%, 90% sequence identity to a primer sequence listed in Table 17.

Labeled Nucleic Acid Reporter Constructs

In some embodiments, the present disclosure provides labeled nucleic acid reporter constructs. In accordance with the present disclosure, cleavage activity (e.g., collateral activity) of a CRISPR/Cas enzyme may be detected by detecting cleavage of an appropriate labeled nucleic acid reporter construct. Typically, a labeled nucleic acid reporter construct for use in accordance with the present disclosure is characterized in that its cleavage can be detected. Those skilled in the art are aware of a variety of strategies for and embodiments of labeled nucleic acid reporter constructs whose collateral cleavage by a particular Cas enzyme is detectable. To give but one example, in some embodiments, a labeled nucleic acid reporter construct may be labeled with a fluorescence-emitting-dye pair (e.g., a FRET pair or a fluor/quencher pair), such that a change (e.g., an increase—such as when cleavage relieves quenching, a decrease, a change in wavelength, or combinations thereof) in fluorescence is observed when the labeled nucleic acid reporter construct is cleaved. Appropriate FRET pairs are known in the art (see, for example, Bajar et al sensors (Basel), 2016; Abraham et al. PLoS One 10:e0134436, 2015).

Various other strategies for detecting cleavage of a labeled nucleic acid reporter construct are also known in the art and include, for example, masking constructs as described with respect to SHERLOCK™ (see, e.g., WO 2018/107129, incorporated herein by reference).

Sample

In some embodiments, methods and compositions of the present disclosure detect target nucleic acids in a sample. In some embodiments a sample is an environmental sample.

In some embodiments, a sample is a biological sample. In some embodiments, a biological sample is collected from a subject (e.g., a human or animal subject). In some embodiments, an animal subject may be a pangolin, bird or a bat. In some embodiments, an animal subject may be a domesticated animal, such as a farm animal or a pet. In some embodiments, an animal subject may be a cat, cow, dog, goat, horse, llama, pig, sheep, etc. In some embodiments, an animal subject may be a rodent. In some embodiments, a subject may be a primate, In some embodiments, a subject may be a human.

In some embodiments, a biological sample is obtained from a subject—e.g., from a fluid or tissue of the subject. In some embodiments, a sample is obtained from a subject by means of a swab, an aspirate, or a lavage. In some embodiments, a sample is obtained from a subject by means of a nasal swab, nasopharyngeal swab, oropharyngeal swab, nasal aspirate, sputum, bronchoalveolar lavage.

In some embodiments, a sample collected using a swab is collected using swabs with a synthetic tip, such as nylon or Dacron®, and an aluminum or plastic shaft. In some embodiments, calcium alginate swabs are not used. In some embodiments, cotton swabs with wooden shafts are not used. In some embodiments, a swab is paces immediately into a sterile tube containing 2-3 ml of viral transport media (i.e. VTM, UTM, M4RT).

In some embodiments a sample is processed. In some embodiments, a sample is processed by dilution, filtration, clarification, distillation, separation; isolation; and/or cryopreservation. In some embodiments, a sample is processed by isolation of specific components. In some embodiments, a sample is processed by isolation of nucleic acid. In some embodiments, RNA is isolated from a sample. In some embodiments, DNA is isolated from a sample. In some embodiments nucleic acid is isolated from a sample using a column. In some embodiments an isolated nucleic acid is diluted after isolation prior to detection of a target nucleic acid. In some embodiments an isolated nucleic acid is serially diluted after isolation isolation prior to detection of a target nucleic acid.

### Limits of Detection

In some embodiments, methods and compositions of the present disclosure provide sensitive detection of a target nucleic acid. In some embodiments, methods and compositions of the present disclosure can detect 3, 5, 7, 9, 11, 13, 15, 17, 19, 21, 23, 25, 27, 29, 31, 33, 35, 37, 39, 41, 43, 45, 47, 49, 51, 53 viral copies of target nucleic acid/μL extracted RNA. In some embodiments, methods and compositions of the present disclosure can detect between 3-11, 5-13, 7-15, 9-17, 11-19, 15-21, 19-23, 21-25, 23-27, 25-31, 27-33, 29-35, 31-37, 33-39, 37-41, 39-45, 45-49, 47-53 viral copies of target nucleic acid/μL extracted RNA.

Formats

In some embodiments, the present disclosure provides particularly useful and/or effective format(s) for detection of SARS-CoV-2.

In some embodiments, nucleic acid isolation may involve, for example, cell disruption, digestion and/or removal of non-nucleic acid cellular components, and/or precipitation of nucleic acid. In some embodiments, reagents for nucleic acid isolation may include thiocyanic acid, compound with guanidine (1:1); Proteinase K; heat; denaturing agents; detergents; carrier RNA (e.g., yeast tRNA). In some embodiments, reagents for nucleic isolation may include phenol/chloroform; BHT; BHA; Surfactin; Capric (8:0); Caprylic (10:0); Lauric acid; Palmitoleic (16:1); Oleic (18:1); Linoleic (18:2); Linolenic (18:3); Arachidonic (20:4); Docosahexaenoic (22:6); Triolein; Monocaprylin; Monocaprin; Monolaurin; Monoolein; Monolinolein; Monolaurin+BHA; Monolaurin+sorbic acid; Decanol; Dodecanol; L-Arginine.

In some embodiments, two or more of target amplification; activation of CRISPR/Cas collateral activity; and detection of signal may be performed in the same reaction vessel. In some embodiments, all of target amplification; activation of CRISPR/Cas collateral activity; and detection of signal are performed in the same reaction vessel.

In some embodiments target amplification involves loop-mediated isothermal amplification (LAMP). Indeed, in some embodiments, the present disclosure provides an insight that LAMP provides certain unexpected advantages relative to alternative amplification technologies (e.g., Nucleic Acid Sequence Based Amplification (NASBA); Strand Displacement Amplification; Recombinase Polymerase Amplification (RPA); Rolling Circle Amplification (RCA)).

In some embodiments, reagents for LAMP may include, for example, Bst 2.0 WarmStart DNA polymerase and WarmStart RTx Reverse transcriptase in a buffer.

### EXAMPLES

### Example 1. General Sherlock SARS-CoV-2 Molecular Diagnostic Assay

The present example describes preparation of diagnostic and detection assays described herein. LAMP primers were obtained from a 100 nmol scale synthesis, using standard desalt purification, and resuspended to 100 μM using nuclease free molecular grade water. A 10×LAMP primer mix was made prior to running the assay. crRNAs were obtained from a 2 nmol scale synthesis, using standard desalt purification, and resuspended to 1 μM using nuclease free molecular grade water.

A 10×LAMP primer Mix was prepared in nuclease-free water. Primer 10× stock solutions of 2 μM F3, 2 μM B3, 16 μM FIP, 16 μM BIP, 4 μM Loop-F, and 4 μM Loop B were prepared. In the 100 uL reaction for SARS-CoV-2 N, the following volumes of the prepared 10× stocks were used: 2 μL of the F3 and B3, 16 μL of the FIP and BIP, and 4 μL of the Loop-F and Loop-B with added 56 μL water. In the 100 μL reaction for SARS-CoV-2 Orf1AB, the following volumes of the prepared 10× stocks were used: 2 μL of the F3 and B3, 16 μL of the FIP and BIP, and 4 μL of the Loop-F with added 60 μL water. In the 100 μL reaction for SARS-CoV-2 RNaseP, the following volumes of the prepared 10× stocks were used: 2 μL of the F3 and B3, 16 μL of the FIP and BIP, and 8 μL of the Loop-F and Loop-B with added 48 μL water.

A carrier RNA was prepared by adding 310 μL RNase-free Water to 310 μg lyophilized Carrier RNA, to obtain 1 μg/L carrier RNA stock solution. A wash buffer was prepared. 60 mL of 96-100% ethanol was added to 15 mL Wash Buffer (WII) concentrate.

A lysis buffer was prepared. The volume of Lysis Buffer/Carrier RNA mix required to process the samples simultaneously was calculated using the following formula: N×0.21 mL (volume of Lysis Buffer/reaction)=A mL, A mL×28 μL/mL=B μL. Where N=number of samples, A=calculated volume of Lysis Buffer (L22), and B=calculated volume of 1 μg/μL Carrier RNA stock solution to add to Lysis Buffer (L22). To 1 μg/μL Carrier RNA stock solution, the volume of Carrier RNA stock solution (B, calculated as above) to the volume of Lysis Buffer (A, calculated as above) was added.

A lysate was prepared. To 25 μL Proteinase K in a microcentrifuge tube, 200 μL of cell-free sample (equilibrated to room temperature) was added. To this tube, 200 μL Lysis Buffer (containing 5.6 μg Carrier RNA) was added and mixed by vortexing at speed 7 to 8 out of 10 for 15 seconds. The tube was incubated in a dry heat block at 56° C. for 15 minutes. Following pulse centrifugation of the sample-lysis mixture tube to remove any drops from the inside of the lid. The Tube was then ready for the binding and washing step.

The prepared RNA/DNA sample was bound and washed by adding 250 μL 96-100% ethanol to the lysate tube to obtain a final ethanol concentration of 37%, followed by vortexing at speed 7-8 out of 10 for 15 seconds. The tube was then incubated for 5 minutes at room temperature (19° C. to 26° C.). The tube was pulse centrifuged to remove any drops from the inside of the lid. The lysate in the ethanol (˜675 μL) was transferred onto a spin column which was subjected to centrifugation at ˜6800×g for 1 minute. The spin column was placed in a clean wash tube and 500 μL Wash Buffer (WII) with ethanol was added to the spin column and subjected to centrifugation at ˜6800×g for 1 minute twice, discarding the collection tube after each centrifugation and discarding the flowthrough. The spin column was dried by centrifugation at >13,000×g. Elution of the RNA/DNA was accomplished by placing the spin column in a clean 1.5-mL recovery tube, and 30 μL of Sterile, RNase-free water was added to the column and incubated at room temperature for about 1 min, then the tubes were subjected to centrifugation at 13,000×g for 1 minute, the eluant contains purified viral nucleic acids.

An amplification reaction having a final volume of 20 uL using LAMP was conducted by preparing a LAMP master mix and 10× primer stock containing the desired primers To the 12 uL LAMP master mix/primer stock, 8 μL of target was added and mixed, spun down. The sample was then placed in a thermocycler/heating block set to 61° C. for 40 minutes.

CRISPR-Cas detection was conducted in a 25 uL volume in a fluorescence microplate at 37° C. A 2 μM RNase alert stock solution was prepared by resuspending individual tubes with 25 μL of nuclease-free water. A Cas Master Mix were prepared. A Cas master mix contained RNase Alert (125 nM), rNTP mix 1 mM, T7 RNA polymerase (1 U/μL), Murine RNase Inhibitor (1 U/μL), LwaCas13a (6.33 ng/μL), crRNA (SARS-CoV-2 N or SARS-CoV-2 Orf1AB or RNaseP) (22.5 nM), and MgCl₂ (9 mM).

20 μL of each Cas Mix was combined with 5 μL of amplified LAMP sample into an 8-tube strip mix, pulse vortexed, and spun down. Then 20 μL from the LAMP-Cas Mix 8 strip tube was added to a 384 Corning black clear bottom well plate and sealed. The plate was placed into the plate 37° C. for 15 minutes with a read at 1-minute intervals.

Data extraction and analysis was performed after the completion of the plate reader run and the data was exported to an excel sheet. For the negative control samples the ratio of the final reading (T15) to the initial reading (T0) for each target analyte and for the positive control samples as well as all patient or contrived samples, was calculated.

Controls were defined as “negative control” when a “no input RNA” reaction was set up as a negative control for amplification. “Positive control” was extracted viral RNA is used as template for LAMP reactions at a concentration of 5000 cp/uL for amplification and detection for each of the SARS-CoV-2 target analytes. Data Analysis and Results Interpretation was conducted such that a sample is considered positive if the final signal is ≥5 fold higher than a valid “no input RNA” sample, and all control assays gave the appropriate results (defined below).

| Result | Interpretation |
|---|---|
| All “Negative Control” sample signals | Negative Control result is “valid” and |
| increase less than 3 folds from the initial | Negative Control signal intensity can be |
| read to the final read (i.e., T0 to T15) | used as background. Test run is valid |
| Any “Negative Control” sample increases ≥3 | Negative Control result is “not valid”; |
| folds from the initial read to the final read | Test run is not valid |
| At T15, signal from all “Positive Control” | Test run is valid |
| samples increase ≥5 fold of valid | |
| “Negative Control” signal | |
| At T15, signal from any “Positive | Test run is not valid |
| control” sample is <5 fold of “Negative | |
| Control” signal | |
| At T15, Patient sample signal is ≥5 fold | Sample is positive for COVID-19 |
| of “Negative Control” signal for one or | |
| both CoV Targets | |
| At T15, Patient sample signal is <5 fold | Sample is negative for COVID-19 |
| of “Negative Control” signal for both | |
| CoV targets AND RNaseP signal is ≥5 | |
| fold of “Negative Control” signal | |
| At T15, Patient sample signal is <5 fold | Test result is not valid and sample |
| of “Negative Control” signal for both | should be retested |
| CoV targets AND RNaseP signal is <5 | |
| fold of “Negative Control” signal | |

### Example 2. Determination and Confirmation of the Limit of Detection (LoD)—Planning

The present example describes preparations for determination of the limit of detection of the SARS-CoV-2 diagnostic.

The SARS-CoV-2 genomic RNA used in the studies originated from a viral culture of SARS-CoV-2 (isolate 2019-nCoV/USA-WA1/2020, MN985325) propagated in Cercopithecus aethiops epithelial kidney cells and stabilized in Trizol. SARS-CoV-2 genomic RNA was purified using PureLink™ Viral DNA/RNA Mini Kit and eluted in 60 μL of nuclease-free water. After quantifying eluted RNA via two independent digital PCR experiments, the concentrated RNA was diluted to 48,000 cp/μL, aliquoted into single use aliquots, stored at −80 C and thawed once immediately before use. This stock of viral RNA was serially diluted in water to create a range of concentrations.

Negative Matrix (NM) was pooled nasopharyngeal swab matrix, collected from 32 symptomatic flu patients, screened by RT-qPCR using the CDC/New York State Department of Health primer probe set (protocol LVD SOP-151.0), and confirmed to be negative for SARS-CoV-2 N1 and N2 target and positive for RNase P was used in this study.

Creation of LoD Panel Members: Each sample tested in this study was created by the addition of 10 microliters of quantified SARS-CoV-2 genomic RNA (positives) or water (negatives) to lysis-treated negative matrix, in order to achieve the desired viral concentration. Ten microliters of viral culture (for contrived clinical positives) or water (for negatives) was added to 200 microliters of the Negative matrix after addition of 225 microliters of PureLink lysis buffer/Proteinase K mixture, and incubation at 56° C. for 15 minutes. This contrived sample was extracted using the PureLink Viral RNA extraction kit, following the manufacturer's instructions with a final elution volume of 30 microliters. Eight microliters of this eluted sample was used as template for each analyte targeted by the CRISPR SARS-CoV-2 Assay (i.e., two SARS-CoV-2 target analytes and the RNaseP control).

Controls were as follows: 1) Extraction Control: RNaseP detection serves as an extraction control in the absence of a SARS-CoV-2 signal. 2) Negative Control: A “no input RNA” reaction was set up as a negative control for amplification and to determine background detection levels for the Cas reaction. This was performed for each LAMP primer set and each guide to be tested. The negative control was created by replacing the 8 ul template volume in the LAMP reaction with an equal volume of nuclease-free water. 3) Positive Control: A positive control for amplification and detection of the SARS-CoV-2 analytes was performed for each Orf1AB and N LAMP primer set and each Orf1AB and N guide to be tested. The positive control was created by replacing the 8 ul template volume in the LAMP reactions with an equal volume of viral RNA extracted from the SARS-CoV-2 Viral RNA Stock Material described above at a concentration of 5000 copies per ul in nuclease free water. The Positive Control was purified using a PureLink Viral RNA extraction kit. Final RNA was eluted in 60 μL of nuclease-free water. Purified viral genomic RNA was quantified by digital PCR and diluted to 5000 copies per microliter in nuclease free water. Positive control aliquots were stored in single use 25 microliter aliquots at a temperature less than negative 70° C. and thawed once immediately before use.

Pooled nasopharyngeal (NP) swab matrix, collected from symptomatic flu patients in the 2019-2020 Flu season and determined to be SARS-CoV-2 negative by RT-qPCR assay using the CDC primer/probe set, was used to perform analytical sensitivity (LoD) studies. LoD was determined by having three operators run the SHERLOCK assay on a series of 7 concentrations of quantified viral material spiked into the pooled NP swab matrix, plus a pooled NP swab matrix without viral material. LoD was confirmed with twenty replicates at the lowest concentration of the series determined to be positive for 3/3 replicates of the worst performing target. Additionally, 20 replicates of 2× the presumptive LoD along with 20 replicates of matrix alone was assayed by SHERLOCK operator. LoD confirmation was run by 4 operators. The final LoD is defined by the lowest concentration displaying at least 19/20 positive replicates for both targets (N and Orf1ab). See Table 2 below. Additionally, if the targets are determined to have different putative LoDs in the initial titration both targets can be confirmed independently if desired, this will involve creating an additional 20 replicates at the lowest concentration of the series determined to be positive for 3/3 replicated of the best performing target.

The Sherlock CRISPR SARS-CoV-2 Test was performed on NP swab samples for every sample processed for the LoD Determination study outlined below.

TABLE 2

| | Analytical | Sub- | Study | 0× | | Concentrations | | | | | | |
|---|---|---|---|---|---|---|---|---|---|---|---|---|
| | Parameter | category | design | con | .25× | .5× | 1× | 1.5× | 2× | 3× | 5× | LOD |
| 1 | LOD | Analytical | Replicates | 3 | 3 | 3 | 3 | 3 | 3 | 3 | 3 | 20 |
| | | Sensitivity | days | 1 | 1 | 1 | 1 | 1 | 1 | 1 | 1 | 1 |
| | | | Operators | 3 | 3 | 3 | 3 | 3 | 3 | 3 | 3 | 4 |

| | Analytical | Sub- | Study | 2× | 3× | | |
|---|---|---|---|---|---|---|---|
| | Parameter | category | design | LOD | LOD | NT | notes |
| 1 | LOD | Analytical | Replicates | 20 | | 20 | Pooled NP clinical negative matrix |
| | | Sensitivity | days | 1 | | 1 | |
| | | | Operators | 4 | | 4 | 4 total operators on 1 day |

LoD Estimation: The viral culture was diluted in nuclease-free water, and then spiked into 200 microliters of the NP matrix AFTER addition of 225 microliters of the PureLink lysis buffer/Proteinase K mixture, and incubation at 56° C. for 15 minutes to achieve concentrations predicted to be 0×, 0.25×, 0.5×, 1×, 1.5×, 2×, 3×, and 5× the LoD based upon previous testing. N=3 replicates of each of the N=7 concentrations along with the Negative matrix only sample was tested by three independent operators. These contrived samples were extracted using the PureLink Viral RNA extraction kit with a final elution volume of 30 μL. 8 μL of this eluate was used as template for each SHERLOCK reaction detecting the CRISPR SARS-CoV-2 Assay target analytes (i.e., N and Orf1AB) as well as the RNaseP control. The estimated LoD for each CRISPR SARS-CoV-2 Assay target analyte was the lowest concentration that is detected as positive for 3 out of 3 replicates.

Sample Extraction: Samples 1-8 were extracted. Sample extraction information for Phase I-LoD Estimation was tracked by the following table 3

TABLE 3

| Sample # | Panel Member ID | Description |
|---|---|---|
| 1 | [initials]-[date]-01 | 5x |
| 2 | [initials]-[date]-02 | 3x |
| 3 | [initials]-[date]-03 | 2x |
| 4 | [initials]-[date]-04 | 1.5x |
| 5 | [initials]-[date]-05 | 1x |
| 6 | [initials]-[date]-06 | 0.5x |
| 7 | [initials]-[date]-07 | 0.25x |
| 8 | [initials]-[date]-08 | 0x |

LAMP Amplification: LAMP reactions was performed according to the layout above using the strip template below for reaction set up.

| Strip | Primer | Position in Strip | | | | | | | |
|---|---|---|---|---|---|---|---|---|---|
| ID | Mix | Well 1 | 2 | 3 | 4 | 5 | 6 | 7 | 8 |
| [initials]- | Orf1AB | Sample | Sample | Sample | Sample | Sample | Sample | Sample | Sample |
| 1 | | 1 | 2 | 3 | 4 | 5 | 6 | 7 | 8 |
| [initials]- | N | Sample | Sample | Sample | Sample | Sample | Sample | Sample | Sample |
| 2 | | 1 | 2 | 3 | 4 | 5 | 6 | 7 | 8 |
| [initials]- | RNaseP | Sample | Sample | Sample | Sample | Sample | Sample | Sample | Sample |
| 3 | | 1 | 2 | 3 | 4 | 5 | 6 | 7 | 8 |
| [initials]- | Orf1AB | Orf1AB | N | <empty> | Orf1AB | N | RNaseP | <empty> | <empty> |
| 4 | or N | Positive | Positive | | Negative | Negative | Negative | | |
| | or | Control | Control | | Control | Control | Control | | |
| | RNaseP | | | | | | | | |

For each extracted sample, one LAMP reaction was performed for each of three primer sets. Additionally, a positive control for LAMP-Cas detection of CoV targets was included (previously extracted viral RNA at 5000 cp/μL). One negative control for LAMP-Cas with water instead of template was performed for each LAMP Primer Set and Cas reaction.

Phase I LoD Estimation/Cas Detection: Cas reactions was set up and performed following the steps listed in the Sherlock CRISPR SARS-CoV-2 Test Instruction. Reactions was performed in a 384 well plate following the template below.

| CasGuide | | | | |
|---|---|---|---|---|
| RNA: | Orf1AB | N | RNaseP | As indicated |
| LAMP | [initials]- | [initials]- | [initials]- | [initials]-4 |
| Strip | 1 | 2 | 3 | |
| ROW/ | 1 | 3 | 5 | 7 |
| Column | | | | |
| A | 8 | 8 | 8 | <empty> |
| C | 7 | 7 | 7 | <empty> |
| E | 6 | 6 | 6 | RNaseP negative |
| | | | | control |
| G | 5 | 5 | 5 | N Negative control |
| I | 4 | 4 | 4 | Orf1AB Negative |
| | | | | control |
| K | 3 | 3 | 3 | <empty> |
| M | 2 | 2 | 2 | N Positive Control |
| O | 1 | 1 | 1 | Orf1AB Positive |
| | | | | Control |

Phase II—LoD Confirmation: Twenty replicates of the estimated LoD (as determined from Phase I testing) or 2× the LoD was spiked into NM. Twenty replicates of matrix alone was assayed simultaneously. Each extraction was tested with the Sherlock™ CRISPR SARS-CoV-2 Test for each of the two SARS-CoV-2 target analytes as well as RNaseP. If ≥19/20 replicates for each of the SARS-CoV-2 targets is positive for SARS-CoV-2, the LoD will have been said to be established. If <19/20 replicates are positive, the study was repeated with at least a 2× higher input of viral RNA until the LoD is determined. Included in all LAMP runs was a positive control as described above and a no template negative control.

For LoD Confirmation Sample information, LAMP set up and Cas set up followed the protocol according to the tables below.

| Sample # | Panel Member ID | Description |
|---|---|---|
| 101 | [initials]-[date]-101 | 1xLoD |
| 102 | [initials]-[date]-102 | 1xLoD |
| 103 | [initials]-[date]-103 | 1xLoD |
| 104 | [initials]-[date]-104 | 1xLoD |
| 105 | [initials]-[date]-105 | 1xLoD |
| 106 | [initials]-[date]-106 | 2xLoD |
| 107 | [initials]-[date]-107 | 2xLoD |
| 108 | [initials]-[date]-108 | 2xLoD |
| 109 | [initials]-[date]-109 | 2xLoD |
| 110 | [initials]-[date]-110 | 2xLoD |
| 111 | [initials]-[date]-111 | NTC |
| 112 | [initials]-[date]-112 | NTC |
| 113 | [initials]-[date]-113 | NTC |
| 114 | [initials]-[date]-114 | NTC |
| 115 | [initials]-[date]-115 | NTC |

| | Primer | | | | | | | | |
|---|---|---|---|---|---|---|---|---|---|
| Strip ID | Mix | Well 1 | 2 | 3 | 4 | 5 | 6 | 7 | 8 |
| [initials]- | Orf1AB | Sample | Sample | Sample | Sample | Sample | <empty> | <empty> | <empty> |
| 01 | | 101 | 102 | 103 | 104 | 105 | | | |
| [initials]- | Orf1AB | Sample | Sample | Sample | Sample | Sample | <empty> | <empty> | <empty> |
| 02 | | 106 | 107 | 108 | 109 | 110 | | | |
| [initials]- | Orf1AB | Sample | Sample | Sample | Sample | Sample | <empty> | <empty> | <empty> |
| 03 | | 111 | 112 | 113 | 114 | 115 | | | |
| [initials]- | Orf1AB | Orf1AB | Orf1AB | <empty> | <empty> | <empty> | <empty> | <empty> | <empty> |
| 04 | | Positive | Negative | | | | | | |
| | | Control | Control | | | | | | |
| [initials]- | N | Sample | Sample | Sample | Sample | Sample | <empty> | <empty> | <empty> |
| 05 | | 101 | 102 | 103 | 104 | 105 | | | |
| [initials]- | N | Sample | Sample | Sample | Sample | Sample | <empty> | <empty> | <empty> |
| 06 | | 106 | 107 | 108 | 109 | 110 | | | |
| [initials]- | N | Sample | Sample | Sample | Sample | Sample | <empty> | <empty> | <empty> |
| 07 | | 111 | 112 | 113 | 114 | 115 | | | |
| [initials]- | N | N | N | <empty> | <empty> | <empty> | <empty> | <empty> | <empty> |
| 08 | | positive | Negative | | | | | | |
| | | control | control | | | | | | |
| [initials]- | RNaseP | Sample | Sample | Sample | Sample | Sample | <empty> | <empty> | <empty> |
| 09 | | 101 | 102 | 103 | 104 | 105 | | | |
| [initials]- | RNaseP | Sample | Sample | Sample | Sample | Sample | <empty> | <empty> | <empty> |
| 10 | | 106 | 107 | 108 | 109 | 110 | | | |
| [initials]- | RNaseP | Sample | Sample | Sample | Sample | Sample | <empty> | <empty> | <empty> |
| 11 | | 111 | 112 | 113 | 114 | 115 | | | |
| [initials]- | RNaseP | <empty> | RNaseP | <empty> | <empty> | <empty> | <empty> | <empty> | <empty> |
| 12 | | | Negative | | | | | | |
| | | | Control | | | | | | |

Statistical/Analysis Methods, Sample Size and Acceptance Criteria:
i) During Phase I (LoD Estimation), the estimated LoD for each CRISPR SARS-CoV-2 Assay target analyte was the lowest concentration that is positive for 3 out of 3 replicates.
ii) During Phase II (LoD Confirmation), the LoD was confirmed if:
   ≥19/20 replicates for each of the CRISPR SARS-CoV-2 Assay target analytes is positive for SARS-CoV-2 detection, and
   20/20 replicates of matrix alone are negative for SARS-CoV-2 detection.

Result Reporting:
a) The negative control reactions must produce a Cas signal that increases less than 3-fold over the course of the 15 minute read (defined by the ratio of T15/T0) for the negative control to be valid for the target.
b) The positive control must be positive for both CoV targets; AND the negative controls must be negative for all three targets for the assay run to be valid.
c) In the event that one or more of the positive or negative controls fails to produce the expected results, the affected assay run must be repeated.
d) A sample is considered positive for a target if the Cas signal increases ≥5-fold at the T15 reading over a valid negative control (“no RNA added”) reaction for that target.
e) A sample is negative for COVID-19 if at T15, a Patient sample signal is <5 fold of the Negative Control signal for both CoV targets AND RNaseP signal is ≥5 fold of Negative Control signal.
f) A sample is invalid if at T15, a Patient sample signal is <5 fold of the Negative Control signal for both CoV targets AND RNaseP signal is <5 fold of Negative Control signal. This sample must be repeated starting at the extraction step.

### Example 3. Determination and Confirmation of the Limit of Detection

### 1. Abstract

The Limit of Detection (LoD) study was performed in two phases. Pooled nasopharyngeal (NP) swab samples (confirmed in a one-step RT-qPCR experiment to be negative for COVID19 using CDC/New York State Department of Health primer and probes) were spiked with either quantitated viral SARS-CoV-2 culture or nuclease-free water and then were processed utilizing the Sherlock™ CRISPR SARS-CoV-2 assay and kit.

In Phase I (“LoD Estimation”), triplicate replicates of limiting dilutions of viral SARS-CoV-2 RNA were extracted in the presence of negative clinical matrix using the PureLink™ Viral RNA/DNA Mini Kit, and the extracted RNA was assayed by the Sherlock™ CRISPR SARS-CoV-2 test for two SARS-CoV-2 target analytes (i.e. ORF1ab and N) as well as an RNase P extraction control. The putative LoD for ORF1ab was 4.5 copies/μL of VTM and the putative LoD of N was 0.9 copies/μL of VTM.

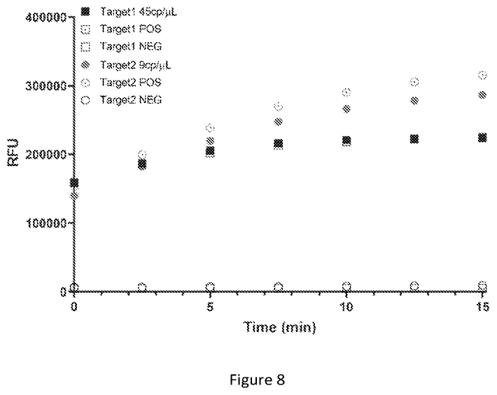

During Phase II (“LoD Confirmation”), LoD was confirmed for ORF1ab and N independently. Twenty (20) replicate samples of the putative 1×LoD concentration for ORF1ab and 20 replicates for 1.5×LoD ORF1ab were tested. Additionally, twenty (20) replicates of a 1×LoD concentration for N and 20 replicates at LoD concentration of 1.5× putative LoD N were assayed simultaneously as described above. See FIG. 8.

The LoD of ORF-1ab was determined to be 6.75 copies/μL of VTM and the LoD of N was determined to be 1.35 copies/μL of VTM. The LoD of the Sherlock™ CRISPR SARS-CoV-02 kit is 6.75 copies/μL.

### 2. Test Method:

a. Test Object:

Negative Matrix (NM): Pooled nasopharyngeal swab matrix, collected from 53 symptomatic flu patients, screened using the CDC/New York State Department of Health primers and probes (LVD SOP-151.0) in a one-step RT-qPCR protocol, and confirmed to be negative for SARS-CoV-2 N1 and N2 target and positive for RNase P was used as the clinical matrix for this study.

Viral genomic RNA from a viral culture of SARS-CoV-2 grown in Vero cell line (stabilized in Trizol) was purified using PureLink™ Viral DNA/RNA Mini Kit and eluted in 60 μL. After quantifying eluted RNA via two independent digital PCR experiments, the concentrated RNA was diluted to 48,000 cp/μL in nuclease free water. This stock of viral RNA was serially diluted in water to create a range of concentrations. Contrived positive samples were generated by spiking in viral dilutions to lysed Negative Matrix (pooled clinical nasopharyngeal samples).

Controls: As described in the LoD Protocol PRO-100-0004 Rev 01, every experimental run of LAMP to Cas reactions contained a positive control for both CoV targets (previously extracted viral RNA at 4,800 copies/μL added directly to the LAMP reaction mix) as well as a negative “no RNA added” control (NTC).

b. Equipment/Instrumentation:

Equipment and instrumentation used in the study are listed in Example 5.

c. Summary of Protocol Steps (Summarize Test Procedure):

LoD Phase I
Three replicates at LoD concentrations of 0×, 0.25×, 0.5×, 0.75×, 1×, 1.25×, 1.5×, 1.75×, 2×, 3×, and 5× were tested to estimate LoD.
Three operators each performed nucleic acid extraction on 11 samples, and eluted purified RNA in 30 μL of water.
Each operator prepared three 2×RT-LAMP mixes (one for each target: ORF1ab, N, RNase P), enough for 11 samples and 2 controls, and aliquoted 12 μL into strip tubes.
Operators added 8 uL of eluted RNA to corresponding strip tubes and incubated the LAMP reaction for 40 minutes at 61° C.
Each operator prepared three CRISPR-Cas mixes (one for each target: ORF1ab, N, RNase P) containing the corresponding CRISPR guide RNA (crRNA), and aliquoted 20 μL into strip tubes.
Operators sealed the Cas plate and placed it into a plate reader at 37° C. and relative fluorescent signal intensity data was collected at 2.5-minute intervals for a total of 15 minutes.

LoD Phase II
Twenty replicate samples of the estimated 1×N LoD (as determined from Phase I testing) were tested along with 20 replicates at the 1×ORF1ab LoD. Additionally, 20 replicates of the estimated N 1.5×LoD (from Phase 1 testing) and 20 replicates at the 1.5×ORF1ab LoD were tested to confirm the LoD for both SARS-CoV-2 targets (ORF1ab and N). Simultaneously 20 replicates at 0×LoD were tested for both ORF1ab and N. LoD was confirmed for ORF1ab and N independently, as described in Table 4 below.

Phase II LoD Sample Replicates

| | Viral copies | Viral copies | Pre-LoD | # of |
|---|---|---|---|---|
| Target | (cp/μL eluate RNA) | (cp/μL VTM) | estimate | Samples |
| ORF1ab | 45 | 6.75 | 1.5x | 20 |
| ORF1ab | 30 | 4.5 | 1x | 20 |
| N | 9 | 1.35 | 1.5x | 20 |
| N | 6 | 0.9 | 1x | 20 |
| ORF1ab/N | 0 | 0 | 0x | 20 |

Five operators each performed nucleic acid extraction on 20 samples, and eluted purified RNA in 30 μL of water.
Each operator prepared three 2×RT-LAMP mixes (one for each target: ORF1ab, N, RNase P), enough for 20 samples and 2 controls, and aliquoted 12 μL into strip tubes.
Operators added 8 uL of eluted RNA to corresponding strip tubes and incubated the LAMP reaction for 40 minutes at 61° C.
Each operator prepared three CRISPR-Cas mixes (one for each target: ORF1ab, N, RNase P), containing the corresponding CRISPR guide RNA (crRNA), and aliquoted 20 μL into strip tubes.
Operators added 5 uL of completed LAMP reaction to corresponding Cas reaction strip tubes, mixed briefly, and transferred 20 uL of Cas-LAMP reaction to a 384 Corning Black Clear Bottom Well Plate.
Operators sealed the Cas plate and placed into a plate reader at 37° C. and relative fluorescent signal intensity data was collected at 2.5-minute intervals for a total of 10 minutes or 15 minutes.

Results interpretation deviation

| Result | Protocol criteria | Study criteria |
|---|---|---|
| Valid Positive | $\frac{{N}_{t = 15}^{spc}}{{N}_{t = 15}^{ntc}} \geq 5 ,$ | $\frac{{N}_{t = 10}^{spc}}{{N}_{t = 10}^{ntc}} \geq 5 ,$ |
| | $\frac{{O}_{t = 15}^{spc}}{{O}_{t = 15}^{ntc}} \geq 5$ | $\frac{{O}_{t = 10}^{spc}}{{O}_{t = 10}^{ntc}} \geq 5$ |
| Valid Negative | $\frac{{N}_{t = 15}^{ntc}}{{N}_{t = 0}^{ntc}} < 3 ,$ | $\frac{{N}_{t = 10}^{ntc}}{{N}_{t = 0}^{ntc}} < 3 ,$ |
| | $\frac{{O}_{t = 15}^{ntc}}{{O}_{t = 0}^{ntc}} < 3 ,$ | $\frac{{O}_{t = 10}^{ntc}}{{O}_{t = 0}^{ntc}} < 3 ,$ |
| | $\frac{{R}_{t = 15}^{ntc}}{{R}_{t = 0}^{ntc}} < 3$ | $\frac{{R}_{t = 10}^{ntc}}{{R}_{t = 0}^{ntc}} < 3$ |
| Positive for COVID-19 | $\frac{{N}_{t = 15}}{{N}_{t = 15}^{ntc}} \geq 5 OR$ | $\frac{{N}_{t = 10}}{{N}_{t = 10}^{ntc}} \geq 5 OR$ |
| | $\frac{{O}_{t = 15}}{{O}_{t = 15}^{ntc}} \geq 5$ | $\frac{{O}_{t = 10}}{{O}_{t = 10}^{ntc}} \geq 5$ |
| Negative for COVID-19 | $\frac{{N}_{t = 15}}{{N}_{t = 15}^{ntc}} < 5 AND$ | $\frac{{N}_{t = 10}}{{N}_{t = 10}^{ntc}} < 5 AND$ |
| | $\frac{{O}_{t = 15}}{{O}_{t = 15}^{ntc}} < 5 AND$ | $\frac{{O}_{t = 10}}{{O}_{t = 10}^{ntc}} < 5 AND$ |
| | $\frac{{R}_{t = 15}}{{R}_{t = 15}^{ntc}} \geq 5$ | $\frac{{R}_{t = 10}}{{R}_{t = 10}^{ntc}} \geq 5$ |
| Invalid | $\frac{{N}_{t = 15}}{{N}_{t = 15}^{ntc}} < 5 AND$ | $\frac{{N}_{t = 10}}{{N}_{t = 10}^{ntc}} < 5 AND$ |
| | $\frac{{O}_{t = 15}}{{O}_{t = 15}^{ntc}} < 5 AND$ | $\frac{{O}_{t = 10}}{{O}_{t = 10}^{ntc}} < 5 AND$ |
| | $\frac{{R}_{t = 15}}{{R}_{t = 15}^{ntc}} < 5$ | $\frac{{R}_{t = 10}}{{R}_{t = 10}^{ntc}} < 5$ |

| | | |
|---|---|---|
| where, | N = | N target reaction fluorescence |
| | O = | Orf1ab target reaction fluorescence |
| | R = | RNaseP target reaction fluorescence |
| | N^{spc} = | N target positive control reaction fluorescence |
| | O^{spc} = | Orf1ab target positive control reaction fluorescence |
| | N^{ntc} = | N target negative control reaction fluorescence |
| | O^{ntc} = | Orf1ab target negative control reaction fluorescence |
| | R^{ntc} = | RNaseP target negative control reaction fluorescence |
| | t = | reaction time |

This protocol deviation does not change the interpretation of any contrived sample or control result.

### 3. Analysis & Discussion:

Phase 1 LoD
LoD Estimation: Values in Tables 4 to 6 for individual replicate results represent fold increase of indicated target over that of the No Template Control (NTC) at T10. A sample was considered positive for the target if that fold-increase is greater than or equal to 5. The LoD is estimated to be the lowest concentration that is detected as positive for the Sherlock™ CRISPR SARS-CoV-2 assay and kit's target analytes (e.g. N and ORF1ab) in 3/3 replicate samples. A LoD estimation was independently determined for the ORF1ab target (Table 4) and the N target (Table 5). Estimated LoD concentrations that were used in Phase II, LoD confirmation, are shown in Table 4 (ORF1ab) and Table 5 (N) below.

TABLE 4
Summary of ORF1AB LoD Estimation

| Viral Copies | | | ORF1AB | | | |
|---|---|---|---|---|---|---|
| (copies/μL | pre-LoD | | | | | Total |
| extracted RNA) | estimate | | rep 1 | rep 2 | rep 3 | Positive |
| 120 | 5x | LoD | 35.0 | 44.2 | 55.8 | 3/3 |
| 72 | 3x | LoD | 28.7 | 41.2 | 59.4 | 3/3 |
| 48 | 2x | LoD | 28.4 | 42.7 | 56.0 | 3/3 |
| 42 | 1.75x | LoD | 35.9 | 43.9 | 59.4 | 3/3 |
| 36 | 1.5x | LoD | 31.0 | 1.2 | 58.2 | 2/3 |
| 30 | 1.25x | LoD | 41.6 | 44.2 | 57.7 | 3/3 |
| 24 | 1x | LoD | 38.2 | 1.1 | 1.1 | 1/3 |
| 18 | 0.75x | LoD | 36.8 | 36.5 | 65.8 | 3/3 |
| 12 | 0.5x | LoD | 1.3 | 43.0 | 1.0 | 1/3 |
| 6 | 0.25x | LoD | 1.1 | 42.9 | 1.0 | 1/3 |
| 0 | 0x | LoD | 1.0 | 1.1 | 1.1 | 0/3 |

| | | | | | |
|---|---|---|---|---|---|
| n/a | Positive Control | 28.5 | 40.0 | 57.9 | 3/3 |
| n/a | Negative Control | 1.0 | 1.0 | 1.0 | 0/3 |

TABLE 5
Summary of N LoD Estimation

| Viral Copies | | | N | | | |
|---|---|---|---|---|---|---|
| (copies/μL | pre-LoD | | | | | Total |
| extracted RNA) | estimate | | rep 1 | rep 2 | rep 3 | Positive |
| 120 | 5x | LoD | 65.5 | 5.6 | 36.9 | 2/3 |
| 72 | 3x | LoD | 66.0 | 19.0 | 38.2 | 3/3 |
| 48 | 2x | LoD | 60.6 | 19.3 | 36.5 | 3/3 |
| 42 | 1.75x | LoD | 57.4 | 17.7 | 37.2 | 3/3 |
| 36 | 1.5x | LoD | 55.5 | 16.4 | 36.1 | 3/3 |
| 30 | 1.25x | LoD | 55.4 | 18.8 | 38.5 | 3/3 |
| 24 | 1x | LoD | 58.0 | 20.1 | 35.7 | 3/3 |
| 18 | 0.75x | LoD | 49.0 | 21.7 | 42.6 | 3/3 |
| 12 | 0.5x | LoD | 51.5 | 19.1 | 39.8 | 3/3 |
| 6 | 0.25x | LoD | 50.1 | 18.8 | 35.3 | 3/3 |
| 0 | 0x | LoD | 0.8 | 0.9 | 1.0 | 0/3 |

| | | | | | |
|---|---|---|---|---|---|
| n/a | Positive Control | 44.5 | 18.0 | 34.9 | 3/3 |
| n/a | Negative Control | 1.8 | 1.2 | 1.1 | 0/3 |

TABLE 6
RNaseP extraction control summary for LoD Estimation

| Viral Copies | | | RNaseP | | | |
|---|---|---|---|---|---|---|
| (copies/μL | pre-LoD | | | | | Total |
| extracted RNA) | estimate | | rep 1 | rep 2 | rep 3 | Positive |
| 120 | 5x | LoD | 22.2 | 29.8 | 32.9 | 3/3 |
| 72 | 3x | LoD | 23.3 | 28.8 | 33.5 | 3/3 |
| 48 | 2x | LoD | 24.3 | 25.2 | 30.4 | 3/3 |
| 42 | 1.75x | LoD | 24.4 | 12.7 | 31.9 | 3/3 |
| 36 | 1.5x | LoD | 24.1 | 24.6 | 30.0 | 3/3 |
| 30 | 1.25x | LoD | 22.3 | 24.7 | 27.3 | 3/3 |
| 24 | 1x | LoD | 28.7 | 22.7 | 30.5 | 3/3 |
| 18 | 0.75x | LoD | 29.8 | 20.3 | 31.9 | 3/3 |
| 12 | 0.5x | LoD | 26.1 | 22.9 | 33.0 | 3/3 |
| 6 | 0.25x | LoD | 24.5 | 26.3 | 31.7 | 3/3 |
| 0 | 0x | LoD | 20.5 | 24.9 | 29.8 | 3/3 |

| | | | | | |
|---|---|---|---|---|---|
| n/a | Negative Control | 1.1 | 1.3 | 1.3 | 0/3 |

In Phase II, 1×LoD ORF1ab (30 copies/μL extracted RNA) and 1.5×LoD ORF1ab (45 copies/nd extracted RNA) were examined to confirm the LoD by running 20 replicates each. For the N target 1×LoD (6 copies/μL extracted RNA) and 1.5×LoD N (9 copies/μL extracted RNA) were examined to confirm the LoD by running 20 replicates of each, Table 7 below. The LoD was confirmed when ≥19/20 replicates for each of the CRISPR SARS-CoV-2 Assay target analytes was positive for SARS-CoV-2 detection, Table 8 below.

TABLE 7
Results for 1x and 1.5x LoD (ORF1ab and N)

| Viral Copies | | | | | Individual Target Result - | | |
|---|---|---|---|---|---|---|---|
| (copies/μL | | Viral Copies | | pre-LoD | # positive/# replicates | | |
| extracted RNA) | | (copies/μL VTM) | | estimate | ORF1AB | N | RNaseP |
| 45 | (ORF1ab) | 6.75 | (ORF1ab) | 1.5x | 19/20 | 20/20 | 20/20 |
| 9 | (N) | 1.35 | (N) | | | | |
| 30 | (ORF1ab) | 4.5 | (ORF1ab) | 1x | 17/20 | 17/20 | 20/20 |
| 6 | (N) | 0.9 | (N) | | | | |

| | | | | | |
|---|---|---|---|---|---|
| n/a | n/a | Positive | 5/5 | 5/5 | n/a |
| | | Control | | | |
| n/a | n/a | NTC | 0/5 | 0/5 | 0/5 |

TABLE 8
Confirmation of the LoD

| | Viral | | | | | |
|---|---|---|---|---|---|---|
| | copies | Viral | | | | |
| | (cp/μL | copies | | | | |
| | eluate | (cp/μL | Pre-LoD | # of | # of | Detection |
| Target | RNA) | VTM) | estimate | Samples | Detected | Rate (%) |
| ORF1ab | 45 | 6.75 | 1.5× | 20 | 19 | 95 |
| N | 9 | 1.35 | 1.5× | 20 | 20 | 100 |

### 4. Conclusions and Recommendations:

The acceptance criteria for confirmation of LoD was met when ≥19/20 replicates were positive for ORF1ab and N targets. The confirmed LoD for the Sherlock™ CRISPR SARS-CoV-2 assay and kit's N target analyte was determined to be:
9 viral copies/μL extracted RNA
1.35 viral copies/μL VTM sample

The confirmed LoD for the Sherlock™ CRISPR SARS-CoV-2 assay and kit's ORF1ab target analyte was determined to be:
45 viral copies/μL extracted RNA
6.75 viral copies/μL VTM sample

### Example 4. Clinical Evaluation of the Assay Using Contrived Clinical Samples

### 1. Abstract (Summarized Procedure & Results):

In the absence of true clinical samples, the clinical evaluation was performed on contrived positive and negative samples, following the procedure specified in PRO-100-0027 Rev 02, Clinical Evaluation for the Sherlock™ CRISPR SARS-CoV-2 Assay Using Contrived Clinical Samples. Nasopharyngeal swab samples confirmed to be negative for COVID-19 by the CDC/New York State Department of Health RT-qPCR primer/probe set (LVD SOP-151.0) were used either unaltered, or spiked with extracted, quantitated SARS-CoV-2 viral RNA to create contrived negative and positive samples, respectively. A total of 30 contrived positive samples spanning 2×, 3×, and 5× the LoD of the Sherlock CRISPR SARS-CoV-2 assay and kit's orf1ab target analyte and 30 contrived negative samples were processed using the Sherlock CRISPR SARS-CoV-2 Test to determine positive percent agreement (sensitivity) and negative percent agreement (specificity) of the test.

### 2. Purpose:

Determine the positive percent agreement (sensitivity) and negative percent agreement (specificity) performance of the Sherlock™ CRISPR SARS-CoV-2 Test using contrived clinical specimens.

### 3. Test Method:

a. Test Object:

Viral genomic RNA from a viral culture of SARS-CoV-2 (stabilized in Trizol) was purified using PureLink™ Viral DNA/RNA Mini Kit and eluted in 60 μL of nuclease-free water. After quantifying eluted RNA via two independent digital PCR experiments, the concentrated RNA was diluted to 48,000 cp/μL. This stock of viral RNA was serially diluted in water to create a range of concentrations.

Contrived positive samples were generated by spiking viral dilutions into lysed nasopharyngeal matrix. Distinct nasopharyngeal swab matrix clinical specimens were used to create contrived clinical samples for this study. All clinical NP swab samples were screened by RT-qPCR for the presence of SARS-CoV-2 using the CDC/New York State Department of Health RT-qPCR primer/probe set for N1, N2 and RNaseP. All NP swab samples used were confirmed to be negative for SARS-CoV-2 N1 and N2 target and positive for RNase P.

Contrived “Negative” clinical samples were taken from unique NP swab samples, screened by RT-qPCR for the presence of SARS-CoV-2 using the CDC/New York State Department of Health RT-qPCR primer/probe set for N1, N2 and RNaseP. (LVD SOP-151.0), and confirmed to be negative for SARS-CoV-2 N1 and N2 target and positive for RNase P, and used unaltered for this study.

Controls: As described in the LoD Protocol PRO-100-0004 Rev 01, every experimental run of LAMP to Cas reactions contained a positive control for both CoV targets (previously extracted viral RNA at 4,800 copies/μL added directly to the LAMP reaction mix) as well as a negative “no RNA added” control (NTC).

b. Equipment/Instrumentation:

Equipment and instrumentation used in the study are listed in Table 9 below.

| Name | Manufacturer | Model # |
|---|---|---|
| Plate Reader | BioTek | NEO2 |
| Thermocycler | Analytika | Biometra TRIO |
| Thermocycler | Analytika | Biometra TONE |
| Microcentrifuge | Axygen | Axyspin R |
| Microcentrifuge | Ohaus | FC5513 |
| Microcentrifuge | Ohaus | FC5513 |
| Dry Bath/Heat Block | Corning | LSE |

Summary of Protocol Steps:
Samples at ORF1ab LoD concentrations of 0×, 2×, 3×, and 5× (corresponding to 0, 90, 135 and 225 viral copies/μL extracted viral RNA) were tested to determine sensitivity and specificity performance.
Five operators each performed nucleic acid extraction on 12 samples, and eluted purified RNA in 30 μL of nuclease-free water.
Each operator prepared three 2×RT-LAMP mixes (one for each target: orf1ab, N, RNase P), enough for 12 samples and 2 controls, and aliquoted 12 μL into 8-well strip tubes.
Operators added 8 μL of eluted RNA to corresponding 8-well strip tubes and incubated LAMP reaction for 40 minutes at 61° C.
Each operator prepared three CRISPR-Cas mixes (one for each target: orf1ab, N, RNase P) containing the corresponding CRISPR guide RNA (crRNA), and aliquoted 20 μL into 8-well strip tubes.
Operators sealed the Cas plate and placed into a plate reader at 37° C. and relative fluorescent signal intensity, caused by cleavage of RNase Alert upon CRISPR complex activation, data was collected at 2.5-minute intervals for a total of 10 minutes or 15 minutes.

TABLE 10
Results interpretation deviation

| Result | Protocol criteria | Study criteria |
|---|---|---|
| Valid Positive | $\frac{{N}_{t = 15}^{spc}}{{N}_{t = 15}^{ntc}} \geq 5 ,$ | $\frac{{N}_{t = 10}^{spc}}{{N}_{t = 10}^{ntc}} \geq 5 ,$ |
| | $\frac{{O}_{t = 15}^{spc}}{{O}_{t = 15}^{ntc}} \geq 5$ | $\frac{{O}_{t = 10}^{spc}}{{O}_{t = 10}^{ntc}} \geq 5$ |
| Valid Negative | $\frac{{N}_{t = 15}^{ntc}}{{N}_{t = 0}^{ntc}} < 3 ,$ | $\frac{{N}_{t = 10}^{ntc}}{{N}_{t = 0}^{ntc}} < 3 ,$ |
| | $\frac{{O}_{t = 15}^{ntc}}{{O}_{t = 0}^{ntc}} < 3 ,$ | $\frac{{O}_{t = 10}^{ntc}}{{O}_{t = 0}^{ntc}} < 3 ,$ |
| | $\frac{{R}_{t = 15}^{ntc}}{{R}_{t = 0}^{ntc}} < 3$ | $\frac{{R}_{t = 10}^{ntc}}{{R}_{t = 0}^{ntc}} < 3$ |
| Positive for COVID-19 | $\frac{{N}_{t = 15}}{{N}_{t = 15}^{ntc}} \geq 5 OR$ | $\frac{{N}_{t = 10}}{{N}_{t = 10}^{ntc}} \geq 5 OR$ |
| | $\frac{{O}_{t = 15}}{{O}_{t = 15}^{ntc}} \geq 5$ | $\frac{{O}_{t = 10}}{{O}_{t = 10}^{ntc}} \geq 5$ |
| Negative for COVID-19 | $\frac{{N}_{t = 15}}{{N}_{t = 15}^{ntc}} < 5 AND$ | $\frac{{N}_{t = 10}}{{N}_{t = 10}^{ntc}} < 5 AND$ |
| | $\frac{{O}_{t = 15}}{{O}_{t = 15}^{ntc}} < 5 AND$ | $\frac{{O}_{t = 10}}{{O}_{t = 10}^{ntc}} < 5 AND$ |
| | $\frac{{RP}_{t = 15}}{{RP}_{t = 15}^{ntc}} \geq 5$ | $\frac{{RP}_{t = 10}}{{RP}_{t = 10}^{ntc}} \geq 5$ |
| Invalid | $\frac{{N}_{t = 15}}{{N}_{t = 15}^{ntc}} < 5 AND$ | $\frac{{N}_{t = 10}}{{N}_{t = 10}^{ntc}} < 5 AND$ |
| | $\frac{{O}_{t = 15}}{{O}_{t = 15}^{ntc}} < 5 AND$ | $\frac{{O}_{t = 10}}{{O}_{t = 10}^{ntc}} < 5 AND$ |
| | $\frac{{R}_{t = 15}}{{R}_{t = 15}^{ntc}} < 5$ | $\frac{{RP}_{t = 10}}{{RP}_{t = 10}^{ntc}} < 5$ |

| | | |
|---|---|---|
| where, | N = | N target reaction fluorescence |
| | O = | Orf1ab target reaction fluorescence |
| | RP = | RNaseP target reaction fluorescence |
| | N^{spc} = | N target positive control reaction fluorescence |
| | O^{spc} = | Orf1ab target positive control reaction fluorescence |
| | N^{ntc} = | N target negative control reaction fluorescence |
| | O^{ntc} = | Orf1ab target negative control reaction fluorescence |
| | R^{ntc} = | RNaseP target negative control reaction fluorescence |
| | t = | reaction time |

This protocol changes the analysis of sample #10 from the following at n=15 minutes:

$$\frac{{N}_{t = 15}}{{N}_{t = 15}^{n t c}} = 1.3 , \frac{{O}_{t = 15}}{{O}_{t = 15}^{n t c}} = 8.2 , \frac{R {P}_{t = 15}}{R {P}_{t = 15}^{n t c}} = 56.3$$

to the following at n=10 minutes:

$$\frac{{N}_{t = 10}}{{N}_{t = 10}^{n t c}} = 1.2 , \frac{{O}_{t = 10}}{{O}_{t = 10}^{n t c}} = 4.8 , \frac{R {P}_{t = 10}}{R {P}_{t = 10}^{n t c}} = 59.3$$

This protocol deviation results in a more accurate interpretation of sample #10 being a true negative rather than a weak false positive. This protocol deviation does not change the interpretation of any other contrived sample or control result.

### 4. Detailed Test Results (Data):

Ratio calculations for all samples are included in Tables 11-13 below.

TABLE 11
Orf1AB target fluorescence ratios

| | Viral copies | | | | | | |
|---|---|---|---|---|---|---|---|
| | per μL | | | | | | |
| | extracted | Sample | | | | | |
| Sample | RNA | type | rep 1 | rep 2 | rep 3 | rep 4 | rep 5 |
| 1 | 0 | NS | 0.9 | 1.0 | 0.6 | 1.0 | 0.9 |
| 2 | 0 | NS | 1.2 | 1.0 | 0.7 | 1.0 | 0.9 |
| 3 | 90 | 2× | 101.1 | 28.3 | 18.6 | 59.2 | 43.6 |
| 4 | 225 | 5× | 105.9 | 28.6 | 22.1 | 55.1 | 45.3 |
| 5 | 0 | NS | 1.2 | 1.0 | 1.0 | 1.1 | 0.9 |
| 6 | 0 | NS | 1.2 | 0.9 | 1.0 | 1.1 | 0.9 |
| 7 | 90 | 2× | 118.3 | 28.1 | 25.1 | 54.6 | 47.5 |
| 8 | 0 | NS | 1.0 | 0.9 | 1.0 | 1.1 | 0.9 |
| 9 | 135 | 3× | 106.2 | 33.4 | 27.3 | 52.5 | 50.8 |
| 10 | 0 | NS | 1.0 | 1.1 | 4.8 | 1.0 | 1.0 |
| 11 | 90 | 2× | 93.3 | 29.4 | 24.5 | 50.9 | 42.5 |
| 12 | 90 | 2× | 83.2 | 30.1 | 24.5 | 50.7 | 44.3 |
| | | Positive | 99.6 | 30.9 | 22.6 | 48.1 | 53.6 |
| | | Control | | | | | |
| | | Negative | 1.1 | 1.1 | 1.1 | 1.0 | 1.1 |
| | | Control | | | | | |

TABLE 12
N target fluorescence ratios

| | Viral copies | | | | | | |
|---|---|---|---|---|---|---|---|
| | per μL | | | | | | |
| | extracted | Sample | | | | | |
| Sample | RNA | type | rep 1 | rep 2 | rep 3 | rep 4 | rep 5 |
| 1 | 0 | NS | 1.1 | 0.9 | 1.3 | 1.1 | 0.8 |
| 2 | 0 | NS | 1.2 | 0.9 | 1.5 | 1.0 | 0.9 |
| 3 | 90 | 2× | 69.1 | 45.4 | 71.3 | 49.1 | 20.4 |
| 4 | 225 | 5× | 62.6 | 44.0 | 55.6 | 36.0 | 23.0 |
| 5 | 0 | NS | 1.1 | 0.8 | 1.3 | 1.1 | 0.9 |
| 6 | 0 | NS | 1.3 | 0.8 | 1.4 | 1.2 | 0.9 |
| 7 | 90 | 2× | 72.3 | 42.4 | 72.0 | 44.9 | 23.6 |
| 8 | 0 | NS | 1.2 | 0.6 | 1.3 | 1.2 | 0.9 |
| 9 | 135 | 3× | 67.2 | 57.5 | 69.2 | 39.5 | 23.7 |
| 10 | 0 | NS | 1.5 | 1.0 | 1.2 | 1.1 | 1.0 |
| 11 | 90 | 2× | 62.9 | 44.1 | 65.9 | 34.0 | 22.2 |
| 12 | 90 | 2× | 67.2 | 47.6 | 54.8 | 36.2 | 22.2 |
| | | Positive | 61.5 | 45.6 | 70.2 | 30.7 | 20.9 |
| | | Control | | | | | |
| | | Negative | 1.3 | 1.6 | 1.0 | 1.1 | 1.2 |
| | | Control | | | | | |

TABLE 13
RNaseP target fluorescence ratios

| | Viral copies | | | | | | |
|---|---|---|---|---|---|---|---|
| | per μL | | | | | | |
| | extracted | Sample | | | | | |
| Sample | RNA | type | rep 1 | rep 2 | rep 3 | rep 4 | rep 5 |
| 1 | 0 | NS | 28.0 | 34.1 | 42.6 | 29.8 | 10.7 |
| 2 | 0 | NS | 29.2 | 29.1 | 47.2 | 26.1 | 10.8 |
| 3 | 90 | 2× | 29.2 | 24.0 | 41.7 | 1.0 | 10.6 |
| 4 | 225 | 5× | 26.6 | 25.4 | 41.3 | 22.5 | 11.1 |
| 5 | 0 | NS | 30.0 | 26.2 | 44.4 | 26.1 | 10.4 |
| 6 | 0 | NS | 27.2 | 24.8 | 47.7 | 24.0 | 10.6 |
| 7 | 90 | 2× | 30.6 | 24.6 | 55.1 | 26.9 | 10.5 |
| 8 | 0 | NS | 31.5 | 24.5 | 51.8 | 28.3 | 11.3 |
| 9 | 135 | 3× | 32.9 | 51.3 | 64.7 | 24.0 | 9.0 |
| 10 | 0 | NS | 31.5 | 49.1 | 59.3 | 28.4 | 10.8 |
| 11 | 90 | 2× | 25.7 | 41.7 | 33.7 | 23.7 | 7.6 |
| 12 | 90 | 2× | 22.6 | 41.5 | 29.4 | 25.5 | 8.1 |
| | | Negative | 1.3 | 1.3 | 1.3 | 1.3 | 1.0 |
| | | Control | | | | | |

### 5. Analysis & Discussion:

Values in Tables 11 to 13 for individual contrived samples and controls represent the ratio of fluorescence of indicated target reaction over that of the corresponding negative control reaction at t=10 minutes, with the exception of the Negative Control which represents the ratio of the fluorescence of the negative control reaction at t=10 minutes and t=0 minutes. Results interpretation are described in Table 3 above and summarized in Tables 14 and 15 below. Confidence intervals (95%) were calculated using the Wilson score interval.

TABLE 14
Sherlock CRISPR SARS-CoV-2 Test agreement with
expected results by target concentration

| | Number of | % Agreement |
|---|---|---|
| Sample Concentration | samples | (95% confidence interval) |
| 5x LoD | 5 | 100% |
| | | (NA*) |
| 3x LoD | 5 | 100% |
| | | (NA*) |
| 2x LoD | 20 | 100% |
| | | (83.9%-100%) |
| Negative specimens (NS) | 30 | 100% |
| | | (88.6%-100%) |

NA*, confidence intervals not calculated for sample sizes of 5 or less

TABLE 15
Calculation of PPA and NPA for the
Sherlock CRISPR SARS-CoV-2 Test

| | | Contrived | |
|---|---|---|---|
| | | Reference Samples | |
| | | + | − |
| Sherlock CRISPR SARS-CoV-2 Test Result | + | 30 | 0 |
| | − | 0 | 30 |
| Total | | 30 | 30 |

Positive percent agreement (Sensitivity) = 100%
Negative percent agreement (Specificity) = 100%

### 6. Conclusions and Recommendations:

Acceptance criteria of the study were met—specifically,
≥19/20 replicates for the 2×LoD samples are positive for SARS-CoV-2 detection,
10/10 replicates for the combined 3× and 5×LoD samples are positive for SARS-CoV-2 detection, and
30/30 replicates of Negative Specimen (NS) samples are negative for SARS-CoV-2 detection
   The positive percent agreement (sensitivity) of the Sherlock SARS-CoV-2 Test is 100% and the negative percent agreement (specificity) of the Sherlock SARS-CoV-2 Test is 100%.

### Example 5 Equipment and Reagents

The present example provides a list of reagents and equipment useful for performing a Sherlock SARS-CoV-2 Test.

TABLE 16
List of Reaction Reagents

| | | Catalog | Stock | | μL required for | |
|---|---|---|---|---|---|---|
| Reagent | Supplier | Number | Concentration | | 100 reactions | |
| RNAaseP 10x LAMP | Sherlock (see | n/a | 10x | | 200 | μL |
| primer mix | section 7.5.1) | | | | | |
| SARS-CoV-2 N 10x | Sherlock (see | n/a | 10x | | 200 | μL |
| LAMP Primer Mix | section 7.5.1) | | | | | |
| SARS-CoV-2 ORF1AB | Sherlock (see | n/a | 10x | | 200 | μL |
| 10x LAMP Primer Mix | section 7.5.1) | | | | | |
| crRNA | IDT | n/a | 1000 | nM | 56 | μL |
| LwaCas13 enzyme | IDT | n/a | 0.5 | mg/mL | 32 | μL |
| 2x WarmStart RT LAMP | NEB | E1700 | 2X | | 1000 | μL |
| T7 RNA pol | NEB | M0251 | 50 | U/μL | 50 | μL |
| Murine Rnase inhibitor | NEB | M0314 | 40 | U/μL | 62.5 | μL |
| MgCl₂ | ThermoFisher | AM9530G | 1 | M | 23 | μL |
| RNase Alert | IDT | 11-04-03-03 | 2 | μM | 156 | μL |

| | | | | |
|---|---|---|---|---|
| PureLink ™ Viral | ThermoFisher | 12280050 | n/a | n/a |
| RNA/DNA Mini Kit | | | | |

TABLE 17
List of LAMP primer and crRNA sequences

| Target | Description | Sequence |
|---|---|---|
| orf1ab | orf1ab-F3 | TGAAAATAGGACCTGAGCG |
| | | (SEQ ID NO. 72) |
| | orf1ab-B3 | ACACCTAGTCATGATTGCA |
| | | (SEQ ID NO. 73) |
| | orf1ab-FIP | CCAATAGAATGATGCCAACA |
| | | GGCGATAGACGTGCCACATG |
| | | C |
| | | (SEQ ID NO. 74) |
| | orf1ab-BIP | GATTGATGTTCAACAATGGG |
| | | GTTTCATTACCATGGACTTG |
| | | ACAAT |
| | | (SEQ ID NO. 75) |
| | orf1ab- | gaaatTAATACGACTCACTA |
| | LF-T7 | TAGGGAAGTGTCTGAAGCAG |
| | | TGGAAAA |
| | | (SEQ ID NO. 76) |
| | crRNA | gatttagactaccccaaaaa |
| | | cgaaggggactaaaacGATC |
| | | ATGGTTGCTTTGTAGGTTAC |
| | | CTGT |
| | | (SEQ ID NO. 77) |
| N | N-5F3 | GCTTCTACGCAGAAGGGA |
| | | (SEQ ID NO. 78) |
| | N-5B3 | GTGACAGTTTGGCCTTGT |
| | | (SEQ ID NO. 79) |
| | N-5FIP | TACTGCTGCCTGGAGTTGAA |
| | | TTCCTCTTCTCGTTCCTCAT |
| | | C |
| | | (SEQ ID NO. 80) |
| | N-5BIP | GCTTTGCTGCTGCTTGACAG |
| | | TGTTGTTGGCCTTTACCA |
| | | (SEQ ID NO. 81) |
| | N-5LB | ATTGAACCAGCTTGAGAGCA |
| | | AA |
| | | (SEQ ID NO. 82) |
| | N-5LF-T7 | gaaatTAATACGACTCACTA |
| | | TAGGGCTTGAACTGTTGCGA |
| | | CTACGT |
| | | (SEQ ID NO. 83) |
| | crRNA | gatttagactaccccaaaaa |
| | | cgaaggggactaaaacGGTG |
| | | ATGCTGCTCTTGCTTTGCTG |
| | | CTGC |
| | | (SEQ ID NO. 84) |
| RNaseP | RP-F3 | TTGATGAGCTGGAGCCA |
| POP7* | | (SEQ ID NO. 85) |
| | RP-B3 | CACCCTCAATGCAGAGTC |
| | | (SEQ ID NO. 86) |
| | RP-FIP | GTGTGACCCTGAAGACTCGG |
| | | TTTTAGCCACTGACTCGGAT |
| | | C |
| | | (SEQ ID NO. 87) |
| | RP-BIP | CCTCCGTGATATGGCTCTTC |
| | | GTTTTTTTCTTACATGGCTC |
| | | TGGTC |
| | | (SEQ ID NO. 88) |
| | RP-LF | ATGTGGATGGCTGAGTTGTT |
| | | (SEQ ID NO. 89) |
| | RP-LB-T7 | GAATTAATACGACTCACTAT |
| | | AGGGCATGCTGAGTACTGGA |
| | | CCTC |
| | | (SEQ ID NO. 90) |
| | crRNA | gatttagactaccccaaaaa |
| | | cgaaggggactaaaacAGTG |
| | | GAGGAGTGTCTTTTCAATTA |
| | | CTTG |
| | | (SEQ ID NO. 91) |

* RNaseP POP7 LAMP primers published in Curtis et al. (2018) J Virol Methods. 255:91-97

TABLE 18
List of Additional Materials & Supplies
Name

0.2 mL strip tubes
1.5 mL snap cap tubes
Molecular grade water (DNase/RNase free)
Filter pipette tips
384 Corning Black Clear Bottom Low Volume Plate
Plate Optical Seal
Extracted RNA from clinical sample/synthetic RNA target
Serological Pipettes

TABLE 19
List of Equipment/Instrumentation

| Item | Manufacturer | Model Number | SHLK# |
|---|---|---|---|
| Microcentrifuge | Axygen | Axyspin R | SHLK-0031 |
| Microcentrifuge | Ohaus | — | SHLK-0033 |
| PCR Workstation-Dead Air Box | Air Clean | AC632DBC | SHLK-0034 |
| PCR Workstation | Air Clean | AC648DBC | SHLK-0035 |
| PCR Workstation | Air Clean | AC648DBC | SHLK-0036 |
| PCR Workstation | Air Clean | AC648DBC | SHLK-0037 |
| PCR Workstation-Dead Air Box | Air Clean | AC632DBC | SHLK-0038 |
| PCR Workstation-Dead Air Box | Air Clean | AC632DBC | SHLK-0039 |
| PCR Workstation | Air Clean | AC648DBC | SHLK-0040 |
| PCR Workstation-Dead Air Box | Air Clean | AC632DBC | SHLK-0041 |
| Plate Reader | BioTek | NEO2 | SHLK-0045 |
| Plate Reader | BioTek | NEO2 | SHLK-0046 |
| Dry Bath/Heat Block | Corning | LSE | SHLK-0047 |
| PCR Workstation-Dead Air Box | Air Clean | AC632DBC | SHLK-0051 |
| PCR Workstation | Air Clean | AC648DBC | SHLK-0052 |
| PCR Machine (3x 48well) | Analytika | Biometra TRIO | SHLK-0056 |
| Biosafety Cabinet Class II Type A2 4 ft | Baker | Sterilgard III | SHLK-0057 |
| Biosafety Cabinet Class II Type A2 4 ft | PHCBI | NHE-N4002A | SHLK-0058 |
| Biosafety Cabinet Class II Type A2 4 ft | PHCBI | NHE-N4002A | SHLK-0059 |
| PCR Workstation | Air Clean | AC648DBC | SHLK-0060 |
| PCR Workstation-Dead Air Box | Air Clean | AC632DBC | SHLK-0061 |
| PCR Workstation | Air Clean | AC648DBC | SHLK-0062 |
| Plate Reader | BioTek | NEO2 | SHLK-0063 |
| PCR Machine (3x 48well) | Analytika | Biometra TRIO | SHLK-0067 |
| PCR Machine (1x 96well) | Analytika | Biometra TONE | SHLK-0068 |
| Tabletop Centrifuge | Beckman | Avanti J15 | SHLK-0069 |
| Biosafety Cabinet Class II Type A2 6 ft | NuAire | NU-425-600 | SHLK-0070 |
| Biosafety Cabinet Class II Type A2 4 ft | NuAire | NU-425-400 | SHLK-0071 |
| PCR Machine (1x 96well) | Analytika | Biometra TONE | SHLK-0072 |
| Microcentrifuge | Ohaus | FC5513 | SHLK-0073 |
| RODI Water Purification System | EMD-Millipore | Milli-Q | SHLK-0075 |
| Vortex | Corning | 6775 | SHLK-0086 |
| Vortex | Ohaus | VXMNAL | SHLK-0087 |
| Minifuge | Ohaus | FC5306 | SHLK-0088 |
| Minifuge | Ohaus | FC5306 | SHLK-0089 |
| Minifuge | Ohaus | FC5306 | SHLK-0090 |
| Minifuge | Ohaus | FC5306 | SHLK-0091 |
| Minifuge | Ohaus | FC5306 | SHLK-0092 |
| Minifuge | Ohaus | FC5306 | SHLK-0093 |
| Minifuge | Ohaus | FC5306 | SHLK-0094 |
| Vortex | Ohaus | VXMNAL | SHLK-0095 |
| Vortex | Ohaus | VXMNAL | SHLK-0096 |
| Vortex | Ohaus | VXMNAL | SHLK-0097 |
| Vortex | Ohaus | VXMNAL | SHLK-0098 |
| Vortex | Ohaus | VXMNAL | SHLK-0099 |
| Vortex | Ohaus | VXMNAL | SHLK-0100 |
| Vortex | Corning | 6775 | SHLK-0101 |
| Vortex | Corning | 6775 | SHLK-0102 |
| Vortex | Corning | 6775 | SHLK-0103 |
| Vortex | Corning | 6775 | SHLK-0104 |
| Minifuge | Corning | 6770 | SHLK-0105 |
| Minifuge | Corning | 6770 | SHLK-0106 |
| Minifuge | Corning | 6770 | SHLK-0107 |
| Vortex | Ohaus | VXMNAL | SHLK-0115 |
| Minifuge | Ohaus | FC5306 | SHLK-0116 |
| Heatblock | Fisher | 14955218 | SHLK-0117 |
| Heatblock | Fisher | 14955218 | SHLK-0118 |
| Single Channel Pipette, P3 (5 each) | Sartorius | — | — |
| Single Channel Pipette, P10 (5 each) | Sartorius | — | — |
| Single Channel Pipette, P20 (5 each) | Sartorius | — | — |
| Single Channel Pipette, P100 (5 each) | Sartorius | — | — |
| Single Channel Pipette, P200 (5 each) | Sartorius | — | — |
| Single Channel Pipette, P300 (5 each) | Sartorius | — | — |
| Single Channel Pipette, P1000 (5 each) | Sartorius | — | — |
| Multi Channel Pipette M100 (2 each) | Sartorius | — | — |
| Multi Channel Pipette M10 (2 each) | Sartorius | — | — |
| Electronic Pipette E10 (4 each) | Sartorius | — | — |
| Electronic Pipette E100 (4 each) | Sartorius | — | — |
| Electronic Pipette E300 (4 each) | Sartorius | — | — |
| Electronic Pipette E1000 (4 each) | Sartorius | — | — |
| Serological Pipette | | | |

### Example 6 LAMP Primer and Guide Polynucleotide Design

The present examples describes a process by which LAMP primers and guide polynucleotides were selected for a Sherlock SARS-CoV-2 Test.

LAMP primers to amplify portions of SARS-CoV-2 were designed using LAMP Primer design software (e.g., PrimerExplorer). Over 80 primer sets covering multiple targets within the SARS-CoV-2 genome were designed. LAMP primers were designed to generate amplicons covering nearly every open reading frame in the SARS-CoV-2 genome including those that are presently used in PCR based SARS-CoV-2 diagnostic or detection systems.

The sequences of LAMP primers generated are shown in Table 20

TABLE 20

| | | | SEQ |
|---|---|---|---|
| | | | ID |
| Name | Sequences | Note | NO. |
| XL-500 | CAACGTGTTGTAGCTTGTC | b1-F3 | 92 |
| XL-501 | ACCATCAGTAGATAAAAGTGCA | b1-B3 | 93 |
| XL-502 | GAACCGCCACACATGACCATCTATAGATTAGCTAATGAGTGTGCT | b1-FIP | 94 |
| XL-503 | GGTGGAACCTCATCAGGAGATGTAACATTGGCCGTGACAG | b1-BIP | 95 |
| XL-504 | CCACAACTGCTTATGCTAATAGTGT | b1-LB | 96 |
| XL-505 | CGTTTCTATAGATTAGCTAATGAGT | b2-F3 | 97 |
| XL-506 | GGCAATTTTGTTACCATCAGT | b2-B3 | 98 |
| XL-507 | GAGGTTCCACCTGGTTTAACATATAGCTCAAGTATTGAGTGAAATGG | b2-FIP | 99 |
| XL-508 | CAGGAGATGCCACAACTGCTTATAGATAAAAGTGCATTAACATTGG | b2-BIP | 100 |
| XL-509 | TAACATTTGTCAAGCTGTCACGG | b2-LB | 101 |
| XL-510 | ATGGCCTCACTTGTTCTT | b3-F3 | 102 |
| XL-511 | TAACATTGGCCGTGACAG | b3-B3 | 103 |
| XL-512 | ACTTGAGCACACTCATTAGCTAATCGCTCGCAAACATACAACG | b3-FIP | 104 |
| XL-513 | GTCATGTGTGGCGGTTCACTACACTATTAGCATAAGCAGTTG | b3-BIP | 105 |
| XL-514 | ACGGTGTGACAAGCTACAACA | b3-LF | 106 |
| XL-515 | ATGTTAAACCAGGTGGAACCTCATC | b3-LB | 107 |
| XL-516 | GCTCGCAAACATACAACG | b4-F3 | 108 |
| XL-517 | GCATTAACATTGGCCGTG | b4-B3 | 109 |
| XL-518 | ACCATTTCACTCAATACTTGAGCAGTAGCTTGTCACACCGTT | b4-FIP | 110 |
| XL-519 | ATATGTTAAACCAGGTGGAACCTCGCTTGACAAATGTTAAAAACACT | b4-BIP | 111 |
| XL-520 | TCAGGAGATGCCACAACTGCTTAT | b4-LB | 112 |
| XL-521 | CCTAACATGCTTAGAATTATGGC | b5-F3 | 113 |
| XL-522 | TAACATTGGCCGTGACAG | b5-B3 | 114 |
| XL-523 | ACTTGAGCACACTCATTAGCTAATCCACTTGTTCTTGCTCGCA | b5-FIP | 115 |
| XL-524 | GTCATGTGTGGCGGTTCACTCACTATTAGCATAAGCAGTTGT | b5-BIP | 116 |
| XL-525 | GACAAGCTACAACACGTTGTATGTT | b5-LF | 117 |
| XL-526 | TAAACCAGGTGGAACCTCATCA | b5-LB | 118 |
| XL-527 | TGTTCTTGCTCGCAAACA | b6-F3 | 119 |
| XL-528 | TAACATTGGCCGTGACAG | b6-B3 | 120 |
| XL-529 | ACTCAATACTTGAGCACACTCATTATACAACGTGTTGTAGCTTGTC | b6-FIP | 121 |
| XL-530 | ATGGTCATGTGTGGCGGTTCCTATTAGCATAAGCAGTTGTGG | b6-BIP | 122 |
| XL-531 | GTTAAACCAGGTGGAACCTCATC | b6-LB | 123 |
| XL-532 | CAACGTGTTGTAGCTTGTC | b7-F3 | 124 |
| XL-533 | ACCATCAGTAGATAAAAGTGCA | b7-B3 | 125 |
| XL-534 | GGTTTAACATATAGTGAACCGCCACGCTAATGAGTGTGCTCAAGT | b7-FIP | 126 |
| XL-535 | GGTGGAACCTCATCAGGAGATGTAACATTGGCCGTGACAG | b7-BIP | 127 |
| XL-536 | CCACAACTGCTTATGCTAATAGTGT | b7-LB | 128 |
| XL-537 | GCTAATGAGTGTGCTCAAGT | b8-F3 | 129 |
| XL-538 | ACTTATCGGCAATTTTGTTACC | b8-B3 | 130 |
| XL-539 | CCTGATGAGGTTCCACCTGGTGAGTGAAATGGTCATGTGT | b8-FIP | 131 |
| XL-540 | GATGCCACAACTGCTTATGCTGTAGATAAAAGTGCATTAACATTGG | b8-BIP | 132 |
| XL-541 | TAACATTTGTCAAGCTGTCACGG | b8-LB | 133 |
| XL-542 | CATGGCTTTGAGTTGACATC | hk1-F3 | 134 |
| XL-543 | ACCTGTAAAACCCCATTGT | hk1-B3 | 135 |
| XL-544 | ATGTGGCACGTCTATCACATAGATGAAGTATTTTGTGAAAATAGGACC | hk1-FIP | 136 |
| XL-545 | TCCACTGCTTCAGACACTTATGCTGAACATCAATCATAAACGGATT | hk1-BIP | 137 |
| XL-546 | TGATGTTCAACAATGGGGT | hk2-F3 | 138 |
| XL-547 | TTAATCTTCAGTTCATCACCAA | hk2-B3 | 139 |
| XL-548 | GCTACATGTGCATTACCATGGACTTTTACAGGTAACCTACAAAGCAA | hk2-FIP | 140 |
| XL-549 | AGTTGTGATGCAATCATGACTAGGTTTATAGGATATTCAATAGTCCAGTC | hk2-BIP | 141 |
| XL-550 | CCACGAGTGCTTTGTTAAGCG | hk2-LB | 142 |
| XL-551 | CCATGATCTGTATTGTCAAGTC | hk3-F3 | 143 |
| XL-552 | GAACTGGGAATTTGTCTGC | hk3-B3 | 144 |
| XL-553 | CGTGGACAGCTAGACACCTAGCATGGTAATGCACATGTAGC | hk3-FIP | 145 |
| XL-554 | GCGTGTTGACTGGACTATTGAATATAACCATGTGTTGAACCTTTC | hk3-BIP | 146 |
| XL-555 | ATGAACTGAAGATTAATGCGGCTTG | hk3-LB | 147 |
| XL-556 | CATCATTCTATTGGATTTGATTACG | hk4-F3 | 148 |
| XL-557 | TCAATAGTCCAGTCAACACG | hk4-B3 | 149 |
| XL-558 | AGATCATGGTTGCTTTGTAGGTTACAATCCGTTTATGATTGATGTTCA | hk4-FIP | 150 |
| XL-559 | GTCAAGTCCATGGTAATGCACATCTTAACAAAGCACTCGTGG | hk4-BIP | 151 |
| XL-560 | TGTGATGCAATCATGACTAGGTGT | hk4-LB | 152 |
| XL-561 | GTCTATAATCCGTTTATGATTGATG | hk5-F3 | 153 |
| XL-562 | CCGCATTAATCTTCAGTTCAT | hk5-B3 | 154 |
| XL-563 | TACATGTGCATTACCATGGACTTGAAACAATGGGGTITTACAGGTA | hk5-FIP | 155 |
| XL-564 | AGTTGTGATGCAATCATGACTAGGTTTATAGGATATTCAATAGTCCAGTC | hk5-BIP | 156 |
| XL-565 | CAGATCATGGTTGCTTTGTAGGT | hk5-LF | 157 |
| XL-566 | AGCTGTCCACGAGTGCTTT | hk5-LB | 158 |
| XL-567 | ACACTTATGCCTGTTGGC | hk6-F3 | 159 |
| XL-568 | ACACGCTTAACAAAGCACT | hk6-B3 | 160 |
| XL-569 | AGGTTACCTGTAAAACCCCATTGTGATTTGATTACGTCTATAATCCGT | hk6-FIP | 161 |
| XL-570 | AAGCAACCATGATCTGTATTGTCATAGACACCTAGTCATGATTGC | hk6-BIP | 162 |
| XL-571 | TGGTAATGCACATGTAGCTAGTTGT | hk6-LB | 163 |
| XL-572 | TGAAAATAGGACCTGAGCG | hk7-F3 | 164 |
| XL-573 | ACACCTAGTCATGATTGCA | hk7-B3 | 165 |
| XL-574 | CCAATAGAATGATGCCAACAGGCGATAGACGTGCCACATGC | hk7-FIP | 166 |
| XL-575 | GATTGATGTTCAACAATGGGGTTTCATTACCATGGACTTGACAAT | hk7-BIP | 167 |
| XL-576 | AAGTGTCTGAAGCAGTGGAAAA | hk7-LF | 168 |
| XL-577 | GGTAACCTACAAAGCAACCATGAT | hk7-LB | 169 |
| XL-578 | AGTCCATGGTAATGCACAT | hk8-F3 | 170 |
| XL-579 | GGGTTACCAATGTCGTGAA | hk8-B3 | 171 |
| XL-580 | GCTTAACAAAGCACTCGTGGAGTAGCTAGTTGTGATGCAATC | hk8-FIP | 172 |
| XL-581 | GATGAACTGAAGATTAATGCGGCTTGAACTGGGAATTTGTCTGC | hk8-BIP | 173 |
| XL-582 | TCAACACATGGTTGTTAAAGCTGCA | hk8-LB | 174 |
| MKW0112 | ATCAGAGGCACGTCAACATC | JP1F3 | 175 |
| MKW0113 | TCACCACTACGACCGTACTG | JP1B3 | 176 |
| MKW0114 | AGGGCTGTTCAAGTTGAGGCAAAGATGGCACTTGTGGCTTAG | JP1FIP | 177 |
| MKW0115 | ACGTTCGGATGCTCGAACTGCATGCCTTCGAGTTCTGCTAC | JP1BIP | 178 |
| MKW0116 | AACGCCTTTTTCAACTTCTA | JP1LF | 179 |
| MKW0117 | AGATGGCACTTGTGGCTTAG | JP2F3 | 180 |
| MKW0118 | CGAAGAAGAACCTTGCGGTA | JP2B3 | 181 |
| MKW0119 | GCAGTTCGAGCATCCGAACGTTGGCGTTTTGCCTCAACTTG | JP2FIP | 182 |
| MKW0120 | TCGAAGGCATTCAGTACGGTCGACTGGTATTTCGCCCACATG | JP2BIP | 183 |
| MKW0121 | GGTGAGACACTTGGTGTCCTTGTC | JP2LB | 184 |
| MKW0122 | CCTCAACTTGAACAGCCCT | JP5F3 | 185 |
| MKW0123 | ACGAAGAAGAACCTTGCGG | JP5B3 | 186 |
| MKW0124 | GCCTTCGAGTTCTGCTACCAGCTCATCAAACGTTCGGATGCT | JP5FIP | 187 |
| MKW0125 | ACGGTCGTAGTGGTGAGACACTTAAGCCACTGGTATTTCGCC | JP5BIP | 188 |
| MKW0126 | TGACCATGAGGTGCAGTTCG | JP5LF | 189 |
| MKW0127 | TGGTGTCCTTGTCCCTCATGT | JP5LB | 190 |
| MKW0128 | CCTCAACTTGAACAGCCCT | JP6F3 | 191 |
| MKW0129 | TATGGCCACCAGCTCCTT | JP6B3 | 192 |
| MKW0130 | CGACCGTACTGAATGCCTTCGACGTTCGGATGCTCGAACTG | JP6FIP | 193 |
| MKW0131 | GACACTTGGTGTCCTTGTCCCTAGAAGAACCTTGCGGTAAGC | JP6BIP | 194 |
| MKW0132 | CATGTGGGCGAAATACCAGTG | JP6LB | 195 |
| MKW0133 | CTTATCAGAGGCACGTCAA | JP9F3 | 196 |
| MKW0134 | TACGACCGTACTGAATGC | JP9B3 | 197 |
| MKW0135 | TCAAGTTGAGGCAAAACGCCTCTTAAAGATGGCACTTGTGG | JP9FIP | 198 |
| MKW0136 | AGCCCTATGTGTTCATCAAACGTTCTTCGAGTTCTGCTACCA | JP9BIP | 199 |
| MKW0137 | TTTTTCAACTTCTACTAAG | JP9LF | 200 |
| MKW0138 | CATCTTAAAGATGGCACTTGT | JP10F3 | 201 |
| MKW0139 | AGTGTCTCACCACTACGA | JP10B3 | 202 |
| MKW0140 | GTTTGATGAACACATAGGGCTGTTGGCTTAGTAGAAGTTGAAAAAGG | JP10FIP | 203 |
| MKW0141 | GTTCGGATGCTCGAACTGCACCGTACTGAATGCCTTCG | JP10BIP | 204 |
| MKW0142 | CAAGTTGAGGCAAAACG | JP10LF | 205 |
| MKW0143 | GATGGCACTTGTGGCTTA | JP11F3 | 206 |
| MKW0144 | AGGACACCAAGTGTCTCA | JP11B3 | 207 |
| MKW0145 | CCGAACGTTTGATGAACACATAGGGTAGAAGTTGAAAAAGGCGT | JP11FIP | 208 |
| MKW0146 | ATGCTCGAACTGCACCTCATCCACTACGACCGTACTGAA | JP11BIP | 209 |
| MKW0147 | GCTGTTCAAGTTGAGGCAAA | JP11LF | 210 |
| MKW0148 | GTAGAAGTTGAAAAAGGCGTT | JP12F3 | 211 |
| MKW0149 | GAAGAAGAACCTTGCGGT | JP12B3 | 212 |
| MKW0150 | ACCATGAGGTGCAGTTCGAGGCCTCAACTTGAACAGCC | JP12FIP | 213 |
| MKW0151 | AGAACTCGAAGGCATTCAGTACGAAGCCACTGGTATTTCGC | JP12BIP | 214 |
| MKW0152 | TCCGAACGTTTGATGAACACATAG | JP12LF | 215 |
| MKW0153 | GTCGTAGTGGTGAGACACTTGG | JP12LB | 216 |
| MKW0154 | TCAAACGTTCGGATGCTC | JP13F3 | 217 |
| MKW0155 | CCAGCTCCTTTATTACCGT | JP13B3 | 218 |
| MKW0156 | CGTACTGAATGCCTTCGAGTTCTGAACTGCACCTCATGGTC | JP13FIP | 219 |
| MKW0157 | GTCGTAGTGGTGAGACACTTGGACGAAGAAGAACCTTGCG | JP13BIP | 220 |
| MKW0158 | GCTACCAGCTCAACCATAACAT | JP13LF | 221 |
| MKW0159 | CTTGTCCCTCATGTGGGCGAA | JP13LB | 222 |
| XL-600 | TGTTATGGAGTGTCTCCTACT | LnS1-F3 | 223 |
| XL-601 | CCAACCTTAGAATCAAGATTGT | LnS1-B3 | 22 |
| XL-602 | CGATTTGTCTGACTTCATCACCTCTAAATGATCTCTGCTTTACTAATGTC | LnS1-FIP | 225 |
| XL-603 | CTCCAGGGCAAACTGGAAAGCAAGCTATAACGCAGCCT | LnS1-BIP | 226 |
| XL-604 | CAACAGAATCTATTGTTAGATTTCC | LnS2-F3 | 227 |
| XL-605 | AGACATTAGTAAAGCAGAGATCA | LnS2-B3 | 228 |
| XL-606 | TTCTCTTCCTGTTCCAAGCATAAACCTTTTGGTGAAGTTTTTAACG | LnS2-FIP | 229 |
| XL-607 | ACTGTGTTGCTGATTATTCTGTCCAATTTAGTAGGAGACACTCCAT | LnS2-BIP | 230 |
| XL-608 | CCGCATCATTTTCCACTTTTAAGTG | LnS2-LB | 231 |
| XL-609 | CAACAGAATCTATTGTTAGATTTCC | LnS3-F3 | 232 |
| XL-610 | AGACATTAGTAAAGCAGAGATCA | LnS3-B3 | 233 |
| XL-611 | GTTCCAAGCATAAACAGATGCAAATCAAACTTGTGCCCTTTTGG | LnS3-FIP | 234 |
| XL-612 | ACTGTGTTGCTGATTATTCTGTCCAATTTAGTAGGAGACACTCCAT | LnS3-BIP | 235 |
| XL-613 | TGATCTCTGCTTTACTAATGTCT | LnS4-F3 | 236 |
| XL-614 | TGAGATTAGACTTCCTAAACAATC | LnS4-B3 | 237 |
| XL-615 | CAGTTTGCCCTGGAGCGATTATGCAGATTCATTTGTAATTAGAGG | LnS4-FIP | 238 |
| XL-616 | TACCAGATGATTTTACAGGCTGCCCACCAACCTTAGAATCAAGA | LnS4-BIP | 239 |
| XL-617 | ACTTTTAAGTGTTATGGAGTGTC | LnS5-F3 | 240 |
| XL-618 | CCAACCTTAGAATCAAGATTGT | LnS5-B3 | 241 |
| XL-619 | CGATTTGTCTGACTTCATCACCTCTTCCTACTAAATTAAATGATCTCTGC | LnS5-FIP | 242 |
| XL-620 | CTCCAGGGCAAACTGGAAAGTCCAAGCTATAACGCAGC | LnS5-BIP | 243 |
| XL-621 | TGTTATGGAGTGTCTCCTACT | LnS6-F3 | 244 |
| XL-622 | CCACCAACCTTAGAATCAAGA | LnS6-B3 | 245 |
| XL-623 | GAGCGATTTGTCTGACTTCATCATGCTTTACTAATGTCTATGCAGAT | LnS6-FIP | 246 |
| XL-624 | GGCAAACTGGAAAGATTGCTGATGTTAGAATTCCAAGCTATAACG | LnS6-BIP | 247 |
| XL-625 | GAGTTGATTTTTGTGGAAAGG | JpS1-F3 | 248 |
| XL-626 | GTTACAAACCAGTGTGTGC | JpS1-B3 | 249 |
| XL-627 | AGGGACATAAGTCACATGCAAGAAGCTATCATCTTATGTCCTTCCCTC | JpS1-FIP | 250 |
| XL-628 | ACAAGAAAAGAACTTCACAACTGCTTTGAAACAAAGACACCTTCA | JpS1-BIP | 251 |
| XL-629 | ACACCATGAGGTGCTGACT | JpS1-LF | 252 |
| XL-630 | TGCCATTTGTCATGATGGAAAAGC | JpS1-LB | 253 |
| XL-631 | GTGACTTATGTCCCTGCA | JpS2-F3 | 254 |
| XL-632 | ACAATTCCTATTACAACATCACAG | JpS2-B3 | 255 |
| XL-633 | ACGAGGAAAGTGTGCTTTTCCAGAAAAGAACTTCACAACTGC | JpS2-FIP | 256 |
| XL-634 | GTTTCAAATGGCACACACTGGTACACAAATGTGTTGTCTGTAG | JpS2-BIP | 257 |
| XL-635 | CCAATTTAATAGTGCTATTGGCA | JpS3-F3 | 258 |
| XL-636 | GCACTTCAGCCTCAACTT | JpS3-B3 | 259 |
| XL-637 | GCATTTTGGTTGACCACATCTTGAAAATTCAAGACTCACTTTCTTCC | JpS3-FIP | 260 |
| XL-638 | GCTTTAAACACGCTTGTTAAACAACTGTCAAGACGTGAAAGGAT | JpS3-BIP | 261 |
| XL-639 | GTTTTCCAAGTGCACTTGCTGT | JpS3-LF | 262 |
| XL-640 | ACAAGAAAAGAACTTCACAACT | JpS5-F3 | 263 |
| XL-641 | TGACAATTCCTATTACAACATCA | JpS5-B3 | 264 |
| XL-642 | GTGCCATTTGAAACAAAGACACCTTGCTCCTGCCATTTGTCAT | JpS5-FIP | 265 |
| XL-643 | ACACTGGTTTGTAACACAAAGGACAGTTACCAGACACAAATGTG | JpS5-BIP | 266 |
| XL-644 | CACGAGGAAAGTGTGCTTTTCCAT | JpS5-LF | 267 |
| XL-645 | GCTATTGGCAAAATTCAAGAC | JpS6-F3 | 268 |
| XL-646 | GCACTTCAGCCTCAACTT | JpS6-B3 | 269 |
| XL-647 | AGCTTGTGCATTTTGGTTGACCTCACTTTCTTCCACAGCA | JpS6-FIP | 270 |
| XL-648 | AACACGCTTGTTAAACAACTTAGCTTGTCAAGACGTGAAAGGAT | JpS6-BIP | 271 |
| XL-649 | CTTATGTCCTTCCCTCAGTC | JpS7-F3 | 272 |
| XL-650 | TGTAGTAATGATTTGTGGTTCAT | JpS7-B3 | 273 |
| XL-651 | GCAGGAGCAGTTGTGAAGTTCTGTAGTCTTCTTGCATGTGACTT | JpS7-FIP | 274 |
| XL-652 | TGTCATGATGGAAAAGCACACTCCTTTGTGTTACAAACCAGTG | JpS7-BIP | 275 |
| XL-653 | CGTGAAGGTGTCTTTGTTTCAAATG | JpS7-LB | 276 |
| XL-654 | ACACAGAATGTTCTCTATGAGA | JpS8-F3 | 277 |
| XL-655 | CGTGAAAGGATATCATTTAAAACAC | JpS8-B3 | 278 |
| XL-656 | ACTTGCTGTGGAAGAAAGTGAGTATTGCCAACCAATTTAATAGTGC | JpS8-FIP | 279 |
| XL-657 | TTGGAAAACTTCAAGATGTGGTCAGCACCAAAATTGGAGCTAAG | JpS8-BIP | 280 |
| XL-658 | TGCACAAGCTTTAAACACGCTTGTT | JpS8-LB | 281 |
| XL-659 | TCAAGACTCACTTTCTTCCA | JpS9-F3 | 282 |
| XL-660 | ATGTCTGCAAACTTTGAAGT | JpS9-B3 | 283 |
| XL-661 | GCGTGTTTAAAGCTTGTGCATTTCAGCAAGTGCACTTGGAA | JpS9-FIP | 284 |
| XL-662 | TGATATCCTTTCACGTCTTGACAAACTGCCTGTGATCAACCTA | JpS9-BIP | 285 |
| XL-663 | TTGAGGCTGAAGTGCAAATTGA | JpS9-LB | 286 |
| XL-664 | ACCTCATGGTGTAGTCTTCT | JpS10-F3 | 287 |
| XL-665 | CACAAATGTGTTGTCTGTAGT | JpS10-B3 | 288 |
| XL-666 | ATGACAAATGGCAGGAGCAGTGCATGTGACTTATGTCCCT | JpS10-FIP | 299 |
| XL-667 | AAGCACACTTTCCTCGTGAAGGTGTGGTTCATAAAAATTCCTTTG | JpS10-BIP | 300 |
| XL-668 | GTTTCAAATGGCACACACTGGTTTG | JpS10-LB | 301 |
| MKW0167 | TCGTGTTGTTTTAGATTTCATCT | N1-1F3 | 302 |
| MKW0168 | TTGAGTGAGAGCGGTGAA | N1-1B3 | 303 |
| MKW0169 | TCCACCAAACGTAATGCGGGCAAACTAAAATGTCTGATAATGGAC | N1-1FIP | 304 |
| MKW0170 | ACTGGCAGTAACCAGAATGGAGCAGTATTATTGGGTAAACCTTG | N1-1BIP | 305 |
| MKW0171 | GTGCATTTCGCTGATTTTGGG | N1-1LF | 306 |
| MKW0172 | ACGAACAAACTAAAATGTCTGA | N1-2F3 | 307 |
| MKW0173 | TTGAGTGAGAGCGGTGAA | N1-2B3 | 308 |
| MKW0174 | TGAATCTGAGGGTCCACCAAAATGGACCCCAAAATCAGC | N1-2FIP | 309 |
| MKW0175 | ACTGGCAGTAACCAGAATGGAGCAGTATTATTGGGTAAACCTTG | N1-2BIP | 310 |
| MKW0176 | CGTAATGCGGGGTGCATTTC | N1-2LF | 311 |
| MKW0177 | GATTTCATCTAAACGAACAAACT | N1-3F3 | 312 |
| MKW0178 | GGGAATTTAAGGTCTTCCTTG | N1-3B3 | 313 |
| MKW0179 | AGGGTCCACCAAACGTAATGCATGTCTGATAATGGACCCCA | N1-3FIP | 314 |
| MKW0180 | GGCGCGATCAAAACAACGTCCATGTTGAGTGAGAGCGG | N1-3BIP | 315 |
| MKW0181 | AAGGTTTACCCAATAATACTGCGTC | N1-3LB | 316 |
| MKW0182 | ACGAACAAACTAAAATGTCTGA | N1-4F3 | 317 |
| MKW0183 | CGAGGGAATTTAAGGTCTTCC | N1-4B3 | 318 |
| MKW0184 | TTACTGCCAGTTGAATCTGAGGGACCCCAAAATCAGCGAAAT | N1-4FIP | 319 |
| MKW0185 | CGATCAAAACAACGTCGGCCTTGCCATGTTGAGTGAGA | N1-4BIP | 320 |
| MKW0186 | AAACGTAATGCGGGGTGC | N1-4LF | 321 |
| MKW0187 | CAAGGTTTACCCAATAATACTGCGT | N1-4LB | 322 |
| MKW0188 | ACTAAAATGTCTGATAATGGACC | N1-5F3 | 323 |
| MKW0189 | CTCGAGGGAATTTAAGGTCT | N1-5B3 | 324 |
| MKW0190 | GTTACTGCCAGTTGAATCTGAGGCCAAAATCAGCGAAATGCA | N1-5FIP | 325 |
| MKW0191 | CGCGATCAAAACAACGTCGGCCATGTTGAGTGAGAGCG | N1-5BIP | 326 |
| MKW0192 | CACCAAACGTAATGCGGGG | N1-5LF | 327 |
| MKW0193 | CAAGGTTTACCCAATAATACTGCGT | N1-5LB | 328 |
| MKW0194 | ACGAACAAACTAAAATGTCTGA | N1-6F3 | 329 |
| MKW0195 | CCTCGAGGGAATTTAAGGT | N1-6B3 | 330 |
| MKW0196 | TTACTGCCAGTTGAATCTGAGGGATGGACCCCAAAATCAGC | N1-6FIP | 331 |
| MKW0197 | GGCCCCAAGGTTTACCCAATCTTCCTTGCCATGTTGAGT | N1-6BIP | 332 |
| MKW0198 | CGTAATGCGGGGTGCATTTC | N1-6LF | 333 |
| MKW0199 | AATACTGCGTCTTGGTTCACCG | N1-6LB | 334 |
| MKW0200 | CAAACTAAAATGTCTGATAATGGAC | N1-7F3 | 335 |
| MKW0201 | CTCGAGGGAATTTAAGGTCT | N1-7B3 | 336 |
| MKW0202 | GTTACTGCCAGTTGAATCTGAGGCCCAAAATCAGCGAAATGC | N1-7FIP | 337 |
| MKW0203 | GGCCCCAAGGTTTACCCAATTCCTTGCCATGTTGAGTG | N1-7BIP | 338 |
| MKW0204 | ACCAAACGTAATGCGGGGT | N1-7LF | 339 |
| MKW0205 | AATACTGCGTCTTGGTTCACC | N1-7LB | 340 |
| MKW0206 | AACACAAGCTTTCGGCAG | N2-1F3 | 341 |
| MKW0207 | TCTTTGTCATCCAATTTGATGG | N2-1B3 | 342 |
| MKW0208 | CGGCCAATGTTTGTAATCAGTTCCCAGAACAAACCCAAGGAAAT | N2-1FIP | 343 |
| MKW0209 | GTTCTTCGGAATGTCGCGCACCTGTGTAGGTCAACCAC | N2-1BIP | 344 |
| MKW0210 | TGATTAGTTCCTGGTCCCCAAA | N2-1LF | 345 |
| MKW0211 | TTGGCATGGAAGTCACACCT | N2-1LB | 346 |
| MKW0212 | AACACAAGCTTTCGGCAG | N2-2F3 | 347 |
| MKW0213 | TTGGATCTTTGTCATCCAATT | N2-2B3 | 348 |
| MKW0214 | CGGCCAATGTTTGTAATCAGTTCCCAGAACAAACCCAAGGAAAT | N2-2FIP | 349 |
| MKW0215 | GTTCTTCGGAATGTCGCGCATGATGGCACCTGTGTAGG | N2-2BIP | 350 |
| MKW0216 | TGATTAGTTCCTGGTCCCCAAA | N2-2LF | 351 |
| MKW0217 | TTGGCATGGAAGTCACACCT | N2-2LB | 352 |
| MKW0218 | TCCAGAACAAACCCAAGG | N2-3F3 | 353 |
| MKW0219 | ATGACTTGATCTTTGAAATTTGG | N2-3B3 | 354 |
| MKW0220 | GCAATTTGCGGCCAATGTTTGAAATTTTGGGGACCAGGA | N2-3FIP | 355 |
| MKW0221 | TGGCATGGAAGTCACACCTTTCCAATTTGATGGCACCTG | N2-3BIP | 356 |
| MKW0222 | CGGGAACGTGGTTGACCT | N2-3LB | 357 |
| MKW0223 | AACACAAGCTTTCGGCAG | N2-4F3 | 358 |
| MKW0224 | GACTTGATCTTTGAAATTTGGATCT | N2-4B3 | 359 |
| MKW0225 | TGCGGCCAATGTTTGTAATCAGCCAAGGAAATTTTGGGGAC | N2-4FIP | 360 |
| MKW0226 | TGGCATGGAAGTCACACCTTATCCAATTTGATGGCACCT | N2-4BIP | 361 |
| MKW0227 | CGGGAACGTGGTTGACCT | N2-4LB | 362 |
| MKW0228 | AACACAAGCTTTCGGCAG | N2-5F3 | 363 |
| MKW0229 | TCTTTGTCATCCAATTTGATGG | N2-5B3 | 364 |
| MKW0230 | TGCGGCCAATGTTTGTAATCAGCAGAACAAACCCAAGGAAAT | N2-5FIP | 365 |
| MKW0231 | GTTCTTCGGAATGTCGCGCACACCTGTGTAGGTCAACC | N2-5BIP | 366 |
| MKW0232 | TGATTAGTTCCTGGTCCCCAAA | N2-5LF | 367 |
| MKW0233 | TTGGCATGGAAGTCACACCT | N2-5LB | 368 |
| MKW0234 | AACACAAGCTTTCGGCAG | N2-6F3 | 369 |
| MKW0235 | GATCTTTGTCATCCAATTTGATG | N2-6B3 | 370 |
| MKW0236 | CGGCCAATGTTTGTAATCAGTTCCAGAACAAACCCAAGGAAAT | N2-6FIP | 371 |
| MKW0237 | GTTCTTCGGAATGTCGCGCACCTGTGTAGGTCAACCAC | N2-6BIP | 372 |
| MKW0238 | TGATTAGTTCCTGGTCCCCAAA | N2-6LF | 373 |
| MKW0239 | TTGGCATGGAAGTCACACCT | N2-6LB | 374 |
| MKW0240 | TAACACAAGCTTTCGGCA | N2-7F3 | 375 |
| MKW0241 | GAAATTTGGATCTTTGTCATCC | N2-7B3 | 376 |
| MKW0242 | GCAATTTGCGGCCAATGTTTGCCAAGGAAATTTTGGGGAC | N2-7FIP | 377 |
| MKW0243 | GTTCTTCGGAATGTCGCGCATTTGATGGCACCTGTGTAG | N2-7BIP | 378 |
| MKW0244 | CAGTTCCTTGTCTGATTAGTTCCTG | N2-7LF | 379 |
| MKW0245 | TTGGCATGGAAGTCACACCT | N2-7LB | 380 |
| MKW0246 | AACACAAGCTTTCGGCAG | N2-8F3 | 381 |
| MKW0247 | CTTTGAAATTTGGATCTTTGTCA | N2-8B3 | 382 |
| MKW0248 | TGCGGCCAATGTTTGTAATCAGCCAAGGAAATTTTGGGGAC | N2-8FIP | 383 |
| MKW0249 | TGGCATGGAAGTCACACCTTTCCAATTTGATGGCACCTG | N2-8BIP | 384 |
| MKW0250 | CGGGAACGTGGTTGACCT | N2-8LB | 385 |
| MKW0251 | AACACAAGCTTTCGGCAG | N2-9F3 | 386 |
| MKW0252 | AGCAAAATGACTTGATCTTTGA | N2-9B3 | 387 |
| MKW0253 | GCAATTTGCGGCCAATGTTTAAGGAAATTTTGGGGACCAG | N2-9FIP | 388 |
| MKW0254 | ATGGAAGTCACACCTTCGGGTCTTTGTCATCCAATTTGATGG | N2-9BIP | 389 |
| MKW0255 | AACGTGGTTGACCTACACAGG | N2-9LB | 390 |
| MKW0256 | GAAATCTGCTGCTGAGGC | N2-10F3 | 391 |
| MKW0257 | AAGGTGTGACTTCCATGC | N2-10B3 | 392 |
| MKW0258 | GGTTTGTTCTGGACCACGTCTAAACGTACTGCCACTAAAGC | N2-10FIP | 393 |
| MKW0259 | ACTGATTACAAACATTGGCCGCGACATTCCGAAGAACGCT | N2-10BIP | 394 |
| MKW0260 | GCCGAAAGCTTGTGTTACATTGTAT | N2-10LF | 395 |
| MKW0261 | TCTTCTCGTTCCTCATCAC | CN-1F3 | 396 |
| MKW0262 | CTTAGAAGCCTCAGCAGC | CN-1B3 | 397 |
| MKW0263 | TCTAGCAGGAGAAGTTCCCCTAGTAGTCGCAACAGTTCAAGAA | CN-1FIP | 398 |
| MKW0264 | CTGCTTGACAGATTGAACCAGCGTGACAGTTTGGCCTTGTT | CN-1BIP | 399 |
| MKW0265 | AAAATGTCTGGTAAAGGCCAACAAC | CN-1LB | 400 |
| MKW0266 | AGTAGGGGAACTTCTCCTG | CN-2F3 | 401 |
| MKW0267 | CTGCCGAAAGCTTGTGTT | CN-2B3 | 402 |
| MKW0268 | GCTGGTTCAATCTGTCAAGCAGTAGAATGGCTGGCAATGG | CN-2FIP | 403 |
| MKW0269 | GCCAACAACAACAAGGCCAAAGTACGTTTTTGCCGAGG | CN-2BIP | 404 |
| MKW0270 | GCAAGAGCAGCATCACCG | CN-2LF | 405 |
| MKW0271 | AGTAGGGGAACTTCTCCTG | CN-3F3 | 406 |
| MKW0272 | CTGCCGAAAGCTTGTGTT | CN-3B3 | 407 |
| MKW0273 | GCTGGTTCAATCTGTCAAGCAGTAGAATGGCTGGCAATGG | CN-3FIP | 408 |
| MKW0274 | AACAAGGCCAAACTGTCACTAACAGTACGTTTTTGCCGAG | CN-3BIP | 409 |
| MKW0275 | GCAAGAGCAGCATCACCG | CN-3LF | 410 |
| MKW0276 | TCCTCATCACGTAGTCGC | CN-4F3 | 411 |
| MKW0277 | GGCTTCTTAGAAGCCTCAG | CN-4B3 | 412 |
| MKW0278 | CCAGCCATTCTAGCAGGAGAAAACAGTTCAAGAAATTCAACTCC | CN-4FIP | 413 |
| MKW0279 | TTGACAGATTGAACCAGCTTGAGACAGCAGATTTCTTAGTGACAGT | CN-4BIP | 414 |
| MKW0280 | GTTCCCCTACTGCTGCCT | CN-4LF | 415 |
| MKW0281 | GCAAAATGTCTGGTAAAGGCCAACA | CN-4LB | 416 |
| MKW0282 | GCTTCTACGCAGAAGGGA | CN-5F3 | 417 |
| MKW0283 | GTGACAGTTTGGCCTTGT | CN-5B3 | 418 |
| MKW0284 | CTACTGCTGCCTGGAGTTGAATTCCTCTTCTCGTTCCTCATC | CN-5FIP | 419 |
| MKW0285 | GCTTTGCTGCTGCTTGACAGTGTTGTTGGCCTTTACCA | CN-5BIP | 420 |
| MKW0286 | CTTGAACTGTTGCGACTACGT | CN-5LF | 421 |
| MKW0287 | ATTGAACCAGCTTGAGAGCAAA | CN-5LB | 422 |
| MKW0288 | GCTGCAATCGTGCTACAACT | CN-8F3 | 423 |
| MKW0289 | TTTGCTCTCAAGCTGGTTCA | CN-8B3 | 424 |
| MKW0290 | TGCGACTACGTGATGAGGAACGTTGCCAAAAGGCTTCTACGC | CN-8FIP | 425 |
| MKW0291 | TCTCCTGCTAGAATGGCTGGCATCTGTCAAGCAGCAGCAAAG | CN-8BIP | 426 |
| MKW0292 | TTGACTGCCGCCTCTGC | CN-8LF | 427 |
| | gaaatTAATACGACTCACTATAGGGTGATTAGTTCCTGGTCCCCAAA | N2-2LFT7 | 528 |
| | gaaatTAATACGACTCACTATAGGGACACCATGAGGTGCTGACT | Jps 1LFT7 | 529 |

The primer sets were tested and each set was ranked as either 4: No amplification/Extremely poor amplification; 3: Poor sensitivity and slow amplification OR 2/2 NTC positive; 2: Good sensitivity and slow amplification or poor sensitivity and fast amplification; or 1: Good sensitivity and speed. Good speed: ave 3000<18: ave 30<25; Good sensitivity: 2/2 for 30 cp. The results of LAMP primer screening are demonstrated in Tables 21 and 22.

TABLE 21
LAMP Primers

| Amplicon | | | | | | | Primer Set |
|---|---|---|---|---|---|---|---|
| ID | F3 | B3 | FIP | BIP | LF | LB | ID |
| B1 | XL-500 | XL-501 | XL-502 | XL-503 | | XL-504 | B1 |
| B2 | XL-505 | XL-506 | XL-507 | XL-508 | | XL-509 | B2 |
| B3 | XL-510 | XL-511 | XL-512 | XL-513 | XL-514 | XL-515 | B3 |
| B4 | XL-516 | XL-517 | XL-518 | XL-519 | | XL-520 | B4 |
| B5 | XL-521 | XL-522 | XL-523 | XL-524 | XL-525 | XL-526 | B5 |
| B6 | XL-527 | XL-528 | XL-529 | XL-530 | | XL-531 | B6 |
| B7 | XL-532 | XL-533 | XL-534 | XL-535 | | XL-536 | B7 |
| B8 | XL-537 | XL-538 | XL-539 | XL-540 | | XL-541 | B8 |
| HK1 | XL-542 | XL-543 | XL-544 | XL-545 | | | HK1 |
| HK2 | XL-546 | XL-547 | XL-548 | XL-549 | | XL-550 | HK2 |
| HK3 | XL-551 | XL-552 | XL-553 | XL-554 | | XL-555 | HK3 |
| HK4 | XL-556 | XL-557 | XL-558 | XL-559 | | XL-560 | HK4 |
| HK5 | XL-561 | XL-562 | XL-563 | XL-564 | XL-565 | XL-566 | HK5 |
| HK6 | XL-567 | XL-568 | XL-569 | XL-570 | | XL-571 | HK6 |
| HK7 | XL-572 | XL-573 | XL-574 | XL-575 | XL-576 | XL-577 | HK7 |
| HK8 | XL-578 | XL-579 | XL-580 | XL-581 | | XL-582 | HK8 |
| JP1 | MKW0112 | MKW0113 | MKW0114 | MKW0115 | MKW0116 | | JP1 |
| JP2 | MKW0117 | MKW0118 | MKW0119 | MKW0120 | | MKW0121 | JP2 |
| JP5 | MKW0122 | MKW0123 | MKW0124 | MKW0125 | MKW0126 | MKW0127 | JP5 |
| JP6 | MKW0128 | MKW0129 | MKW0130 | MKW0131 | | MKW0132 | JP6 |
| JP9 | MKW0133 | MKW0134 | MKW0135 | MKW0136 | MKW0137 | | JP9 |
| JP10 | MKW0138 | MKW0139 | MKW0140 | MKW0141 | MKW0142 | | JP10 |
| JP11 | MKW0143 | MKW0144 | MKW0145 | MKW0146 | MKW0147 | | JP11 |
| JP12 | MKW0148 | MKW0149 | MKW0150 | MKW0151 | MKW0152 | MKW0153 | JP12 |
| JP13 | MKW0154 | MKW0155 | MKW0156 | MKW0157 | MKW0158 | MKW0159 | JP13 |
| LnS1 | XL-600 | XL-601 | XL-602 | XL-603 | | | LnS1 |
| LnS2 | XL-604 | XL-605 | XL-606 | XL-607 | | XL-608 | LnS2 |
| LnS3 | XL-609 | XL-610 | XL-611 | XL-612 | | | LnS3 |
| LnS4 | XL-613 | XL-614 | XL-615 | XL-616 | | | LnS4 |
| LnS5 | XL-617 | XL-618 | XL-619 | XL-620 | | | LnS5 |
| LnS6 | XL-621 | XL-622 | XL-623 | XL-624 | | | LnS6 |
| JpS1 | XL-625 | XL-626 | XL-627 | XL-628 | XL-629 | XL-630 | JpS1 |
| JpS2 | XL-631 | XL-632 | XL-633 | XL-634 | | | JpS2 |
| JpS3 | XL-635 | XL-636 | XL-637 | XL-638 | XL-639 | | JpS3 |
| JpS5 | XL-640 | XL-641 | XL-642 | XL-643 | XL-644 | | JpS5 |
| JpS6 | XL-645 | XL-646 | XL-647 | XL-648 | | | JpS6 |
| JpS7 | XL-649 | XL-650 | XL-651 | XL-652 | | XL-653 | JpS7 |
| JpS8 | XL-654 | XL-655 | XL-656 | XL-657 | | XL-658 | JpS8 |
| JpS9 | XL-659 | XL-660 | XL-661 | XL-662 | | XL-663 | JpS9 |
| JpS10 | XL-664 | XL-665 | XL-666 | XL-667 | | XL-668 | JpS10 |
| N1-1 | MKW0167 | MKW0168 | MKW0169 | MKW0170 | MKW0171 | | N1-1 |
| N1-2 | MKW0172 | MKW0173 | MKW0174 | MKW0175 | MKW0176 | | N1-2 |
| N1-3 | MKW0177 | MKW0178 | MKW0179 | MKW0180 | | MKW0181 | N1-3 |
| N1-4 | MKW0182 | MKW0183 | MKW0184 | MKW0185 | MKW0186 | MKW0187 | N1-4 |
| N1-5 | MKW0188 | MKW0189 | MKW0190 | MKW0191 | MKW0192 | MKW0193 | N1-5 |
| N1-6 | MKW0194 | MKW0195 | MKW0196 | MKW0197 | MKW0198 | MKW0199 | N1-6 |
| N1-7 | MKW0200 | MKW0201 | MKW0202 | MKW0203 | MKW0204 | MKW0205 | N1-7 |
| N2-1 | MKW0206 | MKW0207 | MKW0208 | MKW0209 | MKW0210 | MKW0211 | N2-1 |
| N2-2 | MKW0212 | MKW0213 | MKW0214 | MKW0215 | MKW0216 | MKW0217 | N2-2 |
| N2-3 | MKW0218 | MKW0219 | MKW0220 | MKW0221 | | MKW0222 | N2-3 |
| N2-4 | MKW0223 | MKW0224 | MKW0225 | MKW0226 | | MKW0227 | N2-4 |
| N2-5 | MKW0228 | MKW0229 | MKW0230 | MKW0231 | MKW0232 | MKW0233 | N2-5 |
| N2-6 | MKW0234 | MKW0235 | MKW0236 | MKW0237 | MKW0238 | MKW0239 | N2-6 |
| N2-7 | MKW0240 | MKW0241 | MKW0242 | MKW0243 | MKW0244 | MKW0245 | N2-7 |
| N2-8 | MKW0246 | MKW0247 | MKW0248 | MKW0249 | | MKW0250 | N2-8 |
| N2-9 | MKW0251 | MKW0252 | MKW0253 | MKW0254 | | MKW0255 | N2-9 |
| N2-10 | MKW0256 | MKW0257 | MKW0258 | MKW0259 | MKW0260 | | N2-10 |
| CN-1 | MKW0261 | MKW0262 | MKW0263 | MKW0264 | | MKW0265 | CN-1 |
| CN-2 | MKW0266 | MKW0267 | MKW0268 | MKW0269 | MKW0270 | | CN-2 |
| CN-3 | MKW0271 | MKW0272 | MKW0273 | MKW0274 | MKW0275 | | CN-3 |
| CN-4 | MKW0276 | MKW0277 | MKW0278 | MKW0279 | MKW0280 | MKW0281 | CN-4 |
| CN-5 | MKW0282 | MKW0283 | MKW0284 | MKW0285 | MKW0286 | MKW0287 | CN-5 |
| CN-8 | MKW0288 | MKW0289 | MKW0290 | MKW0291 | MKW0292 | | CN-8 |

TABLE 22
Ct values

| Primer Set | | | | | | | | | |
|---|---|---|---|---|---|---|---|---|---|
| ID | ~3000 cp | | ~30 cp | | NTC | | Ave 3000 cp | Ave 30 cp | Ranking |
| B1 | 21.5 | n/d | n/d | 21.6 | 12.0 | 30.2 | 21.5 | 21.6 | 4 |
| B2 | 17.3 | 17.3 | 23.9 | 29.2 | n/d | n/d | 17.3 | 26.6 | 2 |
| B3 | 12.2 | 11.7 | 30.8 | n/d | n/d | n/d | 12.0 | 30.8 | 2 |
| B4 | 19.3 | 20.6 | 33.0 | 22.9 | n/d | n/d | 19.9 | 28.0 | 3 |
| B5 | 18.0 | 16.4 | 29.4 | n/d | n/d | n/d | 17.2 | 29.4 | 2 |
| B6 | 24.9 | 26.2 | n/d | n/d | n/d | n/d | 25.6 | #DIV/0! | 3 |
| B7 | 21.4 | 23.2 | n/d | 37.2 | n/d | n/d | 22.3 | 37.2 | 3 |
| B8 | 18.9 | 19.1 | n/d | n/d | 34.1 | 35.5 | 19.0 | #DIV/0! | 3 |
| HK1 | 37.4 | n/d | n/d | n/d | n/d | n/d | 37.4 | #DIV/0! | 4 |
| HK2 | 26.1 | 34.2 | n/d | n/d | n/d | n/d | 30.1 | #DIV/0! | 3 |
| HK3 | 17.1 | 17.1 | 24.2 | 25.8 | n/d | n/d | 17.1 | 25.0 | 1 |
| HK4 | 19.2 | 18.8 | n/d | n/d | n/d | n/d | 19.0 | #DIV/0! | 2 |
| HK5 | 11.9 | 12.0 | 16.8 | 17.0 | n/d | n/d | 11.9 | 16.9 | 1 |
| HK6 | 20.1 | 19.5 | n/d | n/d | n/d | n/d | 19.8 | #DIV/0! | 2 |
| HK7 | 10.8 | 11.0 | n/d | n/d | n/d | n/d | 10.9 | #DIV/0! | 2 |
| HK8 | 24.4 | 21.5 | 34.6 | n/d | n/d | n/d | 22.9 | 34.6 | 3 |
| JP1 | 20.9 | 21.7 | 30.6 | 26.8 | n/d | n/d | 21.3 | 28.7 | 2 |
| JP2 | 18.4 | 18.4 | 29.1 | 29.3 | n/d | n/d | 18.4 | 29.2 | 2 |
| JP5 | 9.7 | 9.4 | 13.7 | 12.6 | 47.6 | n/d | 9.6 | 13.1 | 1 |
| JP6 | 17.8 | 19.4 | 27.6 | 30.1 | n/d | 39.9 | 18.6 | 28.8 | 2 |
| JP9 | 37.2 | 39.2 | 51.7 | n/d | n/d | n/d | 38.2 | 51.7 | 3 |
| JP10 | 48.8 | 45.9 | n/d | n/d | n/d | n/d | 47.4 | #DIV/0! | 3 |
| JP11 | 31.4 | 30.3 | n/d | 49.9 | n/d | n/d | 30.8 | 49.9 | 3 |
| JP12 | 16.1 | 16.8 | n/d | 21.9 | n/d | n/d | 16.5 | 21.9 | 2 |
| JP13 | 13.6 | 14.4 | 22.9 | 18.6 | n/d | n/d | 14.0 | 20.7 | 2 |
| LnS1 | n/d | n/d | 53.3 | 38.7 | n/d | n/d | #DIV/0! | 46.0 | 4 |
| LnS2 | 27.7 | 26.9 | 33.2 | 44.7 | n/d | n/d | 27.3 | 39.0 | 3 |
| LnS3 | 38.6 | 35.6 | n/d | n/d | n/d | n/d | 37.1 | #DIV/0! | 3 |
| LnS4 | 26.4 | 26.6 | 36.5 | 34.0 | n/d | n/d | 26.5 | 35.2 | 3 |
| LnS5 | n/d | n/d | n/d | n/d | n/d | n/d | #DIV/0! | #DIV/0! | 4 |
| LnS6 | n/d | n/d | n/d | n/d | n/d | 51.1 | #DIV/0! | #DIV/0! | 4 |
| JpS1 | 11.8 | 12.0 | 15.8 | 24.0 | n/d | n/d | 11.9 | 19.9 | 1 |
| JpS2 | 29.2 | 28.5 | 57.8 | 56.3 | n/d | n/d | 28.9 | 57.0 | 3 |
| JpS3 | 21.9 | 22.3 | n/d | 26.3 | n/d | n/d | 22.1 | 26.3 | 3 |
| JpS5 | 17.9 | 18.0 | 26.0 | 21.7 | n/d | n/d | 17.9 | 23.9 | 1 |
| JpS6 | 33.3 | 33.2 | 38.0 | 52.7 | 44.9 | 45.3 | 33.2 | 45.4 | 3 |
| JpS7 | 30.4 | 30.4 | 39.8 | 38.7 | n/d | n/d | 30.4 | 39.3 | 3 |
| JpS8 | 15.7 | 16.5 | 20.9 | 19.3 | n/d | n/d | 16.1 | 20.1 | 1 |
| JpS9 | 20.1 | 19.3 | 26.5 | 26.8 | n/d | n/d | 19.7 | 26.7 | 2 |
| JpS10 | 19.9 | 19.8 | n/d | 27.4 | n/d | n/d | 19.9 | 27.4 | 2 |
| N1-1 | 21.946 | 25.496 | n/d | n/d | n/d | n/d | 23.7 | #DIV/0! | 3 |
| N1-2 | 27.221 | 24.863 | 34.189 | n/d | 31.641 | n/d | 26.0 | 34.2 | 3 |
| N1-3 | 24.853 | 25.655 | n/d | 27.966 | n/d | n/d | 25.3 | 28.0 | 3 |
| N1-4 | 15.157 | 14.626 | 34.418 | n/d | n/d | n/d | 14.9 | 34.4 | 2 |
| N1-5 | 12.444 | 11.89 | n/d | 24.671 | n/d | n/d | 12.2 | 24.7 | 2 |
| N1-6 | 11.749 | 11.83 | 18.703 | 19.939 | n/d | n/d | 11.8 | 19.3 | 1 |
| N1-7 | 12.871 | 13.762 | 21.513 | 29.409 | n/d | n/d | 13.3 | 25.5 | 2 |
| N2-1 | 16.2 | 15.2 | 23.9 | 20.0 | n/d | n/d | 15.7 | 21.9 | 1 |
| N2-2 | 16.5 | 16.6 | 23.0 | 22.5 | n/d | n/d | 16.5 | 22.7 | 1 |
| N2-3 | 19.1 | 18.3 | 25.5 | 23.3 | n/d | n/d | 18.7 | 24.4 | 2 |
| N2-4 | 16.8 | 17.7 | 24.5 | 25.2 | n/d | 29.4 | 17.2 | 24.8 | 1 |
| N2-5 | 15.4 | 14.5 | 20.9 | 18.9 | 34.5 | n/d | 15.0 | 19.9 | 1 |
| N2-6 | 15.9 | 16.1 | 22.8 | 21.2 | n/d | n/d | 16.0 | 22.0 | 1 |
| N2-7 | 11.1 | 10.5 | 16.2 | 14.3 | n/d | n/d | 10.8 | 15.3 | 1 |
| N2-8 | 15.3 | 15.1 | 19.1 | 17.9 | n/d | n/d | 15.2 | 18.5 | 1 |
| N2-9 | 20.1 | 21.9 | 37.7 | 27.9 | n/d | n/d | 21.0 | 32.8 | 2 |
| N2-10 | 27.5 | 26.6 | 38.4 | 32.2 | n/d | 38.3 | 27.1 | 35.3 | 2 |
| CN-1 | 15.9 | 16.4 | 21.8 | 19.1 | 30.1 | 25.0 | 16.1 | 20.4 | 3 |
| CN-2 | n/d | 39.8 | n/d | n/d | n/d | n/d | 39.8 | #DIV/0! | 4 |
| CN-3 | n/d | n/d | n/d | 34.1 | n/d | n/d | #DIV/0! | 34.1 | 4 |
| CN-4 | 12.9 | 13.3 | 18.4 | 17.7 | n/d | n/d | 13.1 | 18.1 | 1 |
| CN-5 | 12.5 | 13.4 | 16.2 | 15.7 | n/d | n/d | 13.0 | 16.0 | 1 |
| CN-8 | 18.5 | 17.8 | 26.2 | 26.7 | n/d | n/d | 18.1 | 26.5 | 2 |

Notably some algorithm designed primers completely failed to amplify target. Thus, these large scale testing assays were required to empirically identify a LAMP primer set useful for amplifying SARS-CoV-2 nucleic acids.

Having identified viable primer sets a guide RNA comprising a crRNA needed to be constructed that binds to the nucleic acid amplified by the LAMP primer set. Between 2 and 5 guides were designed for a given viable LAMP primer set. To design guides, 28 nt regions located between F2 and F1c, F1c and B1c, or B1c and B2 binding regions were selected for guide screening. Guides were designed by available algorithms to have <10 bp overlap with any LAMP primer in the set. After initial primer screening, the potential guides were tested with the Sherlock reaction. The chosen guides showed high signal to noise ratio comparing the signal observed from LAMP amplification from target versus LAMP amplification without target. Designed guides are listed in Table 23

TABLE 23

| | | Designed, | SEQ |
|---|---|---|---|
| | | Not | ID |
| Name | Sequences | Tested | NO |
| XLcr-600 | gatttagactaccccaaaaacgaaggggactaaaacTCTGGTAATTTATAATTATAATCAGCAA | X | 428 |
| XLcr-601 | gatttagactaccccaaaaacgaaggggactaaaacAAAATCATCTGGTAATTTATAATTATAA | X | 429 |
| XLcr-602 | gatttagactaccccaaaaacgaaggggactaaaacCTGGTAATTTATAATTATAATCAGCAAT | X | 430 |
| XLcr-603 | gatttagactaccccaaaaacgaaggggactaaaacACACTTAAAAGTGGAAAATGATGCGGAA | X | 431 |
| XLcr-604 | gatttagactaccccaaaaacgaaggggactaaaacAAAAGTGGAAAATGATGCGGAATTATAT | X | 432 |
| XLcr-605 | gatttagactaccccaaaaacgaaggggactaaaacACACTTAAAAGTGGAAAATGATGCGGAA | X | 433 |
| XLcr-606 | gatttagactaccccaaaaacgaaggggactaaaacTAAAAGTGGAAAATGATGCGGAATTATA | X | 434 |
| XLcr-607 | gatttagactaccccaaaaacgaaggggactaaaacGAAAGATTGCTGATTATAATTATAAATT | X | 435 |
| XLcr-608 | gatttagactaccccaaaaacgaaggggactaaaacTGCGTTATAGCTTGGAATTCTAACAATC | X | 436 |
| XLcr-609 | gatttagactaccccaaaaacgaaggggactaaaacATTACAAATGAATCTGCATAGACATTAG | X | 437 |
| XLcr-610 | gatttagactaccccaaaaacgaaggggactaaaacTCTGGTAATTTATAATTATAATCAGCAA | X | 438 |
| XLcr-611 | gatttagactaccccaaaaacgaaggggactaaaacCCTGTAAAATCATCTGGTAATTTATAAT | X | 439 |
| XLcr-612 | gatttagactaccccaaaaacgaaggggactaaaacGCCTGTAAAATCATCTGGTAATTTATAA | X | 440 |
| XLcr-613 | gatttagactaccccaaaaacgaaggggactaaaacAAAGTGTGCTTTTCCATCATGACAAATG | X | 441 |
| XLcr-614 | gatttagactaccccaaaaacgaaggggactaaaacGTGTGCTTTTCCATCATGACAAATGGCA | X | 442 |
| XLcr-615 | gatttagactaccccaaaaacgaaggggactaaaacTGGTTCATAAAAATTCCTTTGTGTTACA | X | 443 |
| XLcr-616 | gatttagactaccccaaaaacgaaggggactaaaacATTTGTGGTTCATAAAAATTCCTTTGTG | X | 444 |
| XLcr-617 | gatttagactaccccaaaaacgaaggggactaaaacCATTTAAAACACTTGAAATTGCACCAAA | X | 445 |
| XLcr-618 | gatttagactaccccaaaaacgaaggggactaaaacAAACACTTGAAATTGCACCAAAATTGGA | X | 446 |
| XLcr-619 | gatttagactaccccaaaaacgaaggggactaaaacTCTGTAGTAATGATTTGTGGTTCATAAA | X | 447 |
| XLcr-620 | gatttagactaccccaaaaacgaaggggactaaaacTGATGGAAAAGCACACTTTCCTCGTGAA | X | 448 |
| XLcr-621 | gatttagactaccccaaaaacgaaggggactaaaacAAGTGCACTTGGAAAACTTCAAGATGTG | X | 449 |
| XLcr-622 | gatttagactaccccaaaaacgaaggggactaaaacCATTTAAAACACTTGAAATTGCACCAAA | X | 450 |
| XLcr-623 | gatttagactaccccaaaaacgaaggggactaaaacCCATTTGAAACAAAGACACCTTCACGAG | X | 451 |
| XLcr-624 | gatttagactaccccaaaaacgaaggggactaaaacGTGCCATTTGAAACAAAGACACCTTCAC | X | 452 |
| XLcr-625 | gatttagactaccccaaaaacgaaggggactaaaacACAAGCGTGTTTAAAGCTTGTGCATTTT | X | 453 |
| XLcr-626 | gatttagactaccccaaaaacgaaggggactaaaacGCACCAAAATTGGAGCTAAGTTGTTTAA | X | 454 |
| XLcr-627 | gatttagactaccccaaaaacgaaggggactaaaacTGAAATTGCACCAAAATTGGAGCTAAGT | X | 455 |
| XLcr-628 | gatttagactaccccaaaaacgaaggggactaaaacCCAGTGTGTGCCATTTGAAACAAAGACA | X | 456 |
| XLcr-629 | gatttagactaccccaaaaacgaaggggactaaaacACAAACCAGTGTGTGCCATTTGAAACAA | X | 457 |
| MKW0293 | gatttagactaccccaaaaacgaaggggactaaaacCCGACGTTGTTTTGATCGCGCCCCACTG | X | 458 |
| MKW0294 | gatttagactaccccaaaaacgaaggggactaaaacGTGGGGCGCGATCAAAACAACGTCGGCC | X | 459 |
| MKW0295 | gatttagactaccccaaaaacgaaggggactaaaacGAACGCAGTGGGGCGCGATCAAAACAAC | X | 460 |
| MKW0296 | gatttagactaccccaaaaacgaaggggactaaaacGGCAGTAACCAGAATGGAGAACGCAGTG | X | 461 |
| MKW0297 | gatttagactaccccaaaaacgaaggggactaaaacCGCCCCACTGCGTTCTCCATTCTGGTTA | — | 462 |
| MKW0298 | gatttagactaccccaaaaacgaaggggactaaaacGCTGAAGCGCTGGGGGCAAATTGTGCAA | — | 463 |
| MKW0299 | gatttagactaccccaaaaacgaaggggactaaaacGAAGCGCTGGGGGCAAATTGTGCAATTT | — | 464 |
| MKW0300 | gatttagactaccccaaaaacgaaggggactaaaacCAATTTGCCCCCAGCGCTTCAGCGTTCT | — | 465 |
| MKW0301 | gatttagactaccccaaaaacgaaggggactaaaacCCCCCAGCGCTTCAGCGTTCTTCGGAAT | — | 466 |
| MKW0302 | gatttagactaccccaaaaacgaaggggactaaaacAAATTGCACAATTTGCCCCCAGCGCTTC | — | 467 |
| MKW0303 | gatttagactaccccaaaaacgaaggggactaaaacAATGGCGGTGATGCTGCTCTTGCTTTGC | — | 468 |
| MKW0304 | gatttagactaccccaaaaacgaaggggactaaaacGCAAAGCAAGAGCAGCATCACCGCCATT | — | 469 |
| MKW0305 | gatttagactaccccaaaaacgaaggggactaaaacTGAGAGCAAAATGTCTGGTAAAGGCCAA | X | 470 |
| MKW0306 | gatttagactaccccaaaaacgaaggggactaaaacGGTGATGCTGCTCTTGCTTTGCTGCTGC | — | 471 |
| MKW0307 | gatttagactaccccaaaaacgaaggggactaaaacGCAGCAGCAAAGCAAGAGCAGCATCACC | — | 472 |
| XLcr-500 | gatttagactaccccaaaaacgaaggggactaaaacTGACAAATGTTAAAAACACTATTAGCAT | X | 473 |
| XLcr-501 | gatttagactaccccaaaaacgaaggggactaaaacAATGTTAAAAACACTATTAGCATAAGCA | X | 474 |
| XLcr-502 | gatttagactaccccaaaaacgaaggggactaaaacCAAATGTTAAAAACACTATTAGCATAAG | X | 475 |
| XLcr-503 | gatttagactaccccaaaaacgaaggggactaaaacTGACAGCTTGACAAATGTTAAAAACACT | X | 476 |
| XLcr-504 | gatttagactaccccaaaaacgaaggggactaaaacCGTGACAGCTTGACAAATGTTAAAAACA | X | 477 |
| XLcr-505 | gatttagactaccccaaaaacgaaggggactaaaacTGTTGTAGCTTGTCACACCGTTTCTATA | — | 478 |
| XLcr-506 | gatttagactaccccaaaaacgaaggggactaaaacCATCTCCTGATGAGGTTCCACCTGGTTT | — | 479 |
| XLcr-507 | gatttagactaccccaaaaacgaaggggactaaaacCTCCTGATGAGGTTCCACCTGGTTTAAC | — | 480 |
| XLcr-508 | gatttagactaccccaaaaacgaaggggactaaaacTAGCATAAGCAGTTGTGGCATCTCCTGA | X | 481 |
| XLcr-509 | gatttagactaccccaaaaacgaaggggactaaaacTAGAAACGGTGTGACAAGCTACAACACG | — | 482 |
| XLcr-510 | gatttagactaccccaaaaacgaaggggactaaaacACGGTGTGACAAGCTACAACACGTTGTA | — | 483 |
| XLcr-511 | gatttagactaccccaaaaacgaaggggactaaaacCATCTCCTGATGAGGTTCCACCTGGTTT | — | 484 |
| XLcr-512 | gatttagactaccccaaaaacgaaggggactaaaacCCTGATGAGGTTCCACCTGGTTTAACAT | — | 485 |
| XLcr-513 | gatttagactaccccaaaaacgaaggggactaaaacCCTGATGAGGTTCCACCTGGTTTAACAT | X | 486 |
| XLcr-514 | gatttagactaccccaaaaacgaaggggactaaaacTGAGGTTCCACCTGGTTTAACATATAGT | X | 487 |
| XLcr-515 | gatttagactaccccaaaaacgaaggggactaaaacTGACAAATGTTAAAAACACTATTAGCAT | X | 488 |
| XLcr-516 | gatttagactaccccaaaaacgaaggggactaaaacAATGTTAAAAACACTATTAGCATAAGCA | X | 489 |
| XLcr-517 | gatttagactaccccaaaaacgaaggggactaaaacTGACAGCTTGACAAATGTTAAAAACACT | X | 490 |
| XLcr-518 | gatttagactaccccaaaaacgaaggggactaaaacCAGCTTGACAAATGTTAAAAACACTATT | X | 491 |
| XLcr-519 | gatttagactaccccaaaaacgaaggggactaaaacAATCAAATCCAATAGAATGATGCCAACA | X | 492 |
| XLcr-520 | gatttagactaccccaaaaacgaaggggactaaaacATCAAATCCAATAGAATGATGCCAACAG | X | 493 |
| XLcr-521 | gatttagactaccccaaaaacgaaggggactaaaacTAATCAAATCCAATAGAATGATGCCAAC | X | 494 |
| XLcr-522 | gatttagactaccccaaaaacgaaggggactaaaacACACGCTTAACAAAGCACTCGTGGACAG | X | 495 |
| XLcr-523 | gatttagactaccccaaaaacgaaggggactaaaacCGCTTAACAAAGCACTCGTGGACAGCTA | X | 496 |
| XLcr-524 | gatttagactaccccaaaaacgaaggggactaaaacCCGCATTAATCTTCAGTTCATCACCAAT | X | 497 |
| XLcr-525 | gatttagactaccccaaaaacgaaggggactaaaacACAAGCCGCATTAATCTTCAGTTCATCA | X | 498 |
| XLcr-526 | gatttagactaccccaaaaacgaaggggactaaaacACACCTAGTCATGATTGCATCACAACTA | X | 499 |
| XLcr-527 | gatttagactaccccaaaaacgaaggggactaaaacAGACACCTAGTCATGATTGCATCACAAC | X | 500 |
| XLcr-528 | gatttagactaccccaaaaacgaaggggactaaaacCAGCTAGACACCTAGTCATGATTGCATC | X | 501 |
| XLcr-529 | gatttagactaccccaaaaacgaaggggactaaaacACCTACAAAGCAACCATGATCTGTATTG | — | 502 |
| XLcr-530 | gatttagactaccccaaaaacgaaggggactaaaacACACGCTTAACAAAGCACTCGTGGACAG | — | 503 |
| XLcr-531 | gatttagactaccccaaaaacgaaggggactaaaacACGCTTAACAAAGCACTCGTGGACAGCT | — | 504 |
| XLcr-532 | gatttagactaccccaaaaacgaaggggactaaaacTCACAACTAGCTACATGTGCATTACCAT | X | 505 |
| XLcr-533 | gatttagactaccccaaaaacgaaggggactaaaacCACAACTAGCTACATGTGCATTACCATG | — | 506 |
| XLcr-534 | gatttagactaccccaaaaacgaaggggactaaaacATTTGATTACGTCTATAATCCGTTTATG | — | 507 |
| XLcr-535 | gatttagactaccccaaaaacgaaggggactaaaacGATCATGGTTGCTTTGTAGGTTACCTGT | — | 508 |
| XLcr-536 | gatttagactaccccaaaaacgaaggggactaaaacCAGATCATGGTTGCTTTGTAGGTTACCT | — | 509 |
| XLcr-537 | gatttagactaccccaaaaacgaaggggactaaaacGCTTTTCCACTGCTTCAGACACTTATGC | — | 510 |
| XLcr-538 | gatttagactaccccaaaaacgaaggggactaaaacATTATAGGATATTCAATAGTCCAGTCAA | X | 511 |
| XLcr-539 | gatttagactaccccaaaaacgaaggggactaaaacCCAATTATAGGATATTCAATAGTCCAGT | X | 512 |
| XLcr-540 | gatttagactaccccaaaaacgaaggggactaaaacGCTTTAACAACCATGTGTTGAACCTTTC | X | 513 |
| XLcr-541 | gatttagactaccccaaaaacgaaggggactaaaacGCAGCTTTAACAACCATGTGTTGAACCT | X | 514 |
| XLcr-542 | gatttagactaccccaaaaacgaaggggactaaaacCTTTAACAACCATGTGTTGAACCTTTCT | X | 515 |
| MKW0160 | gatttagactaccccaaaaacgaaggggactaaaacACCTCATGGTCATGTTATGGTTGAGCTG | — | 516 |
| MKW0161 | gatttagactaccccaaaaacgaaggggactaaaacCAGCTCAACCATAACATGACCATGAGGT | — | 517 |
| MKW0162 | gatttagactaccccaaaaacgaaggggactaaaacTGGTCATGTTATGGTTGAGCTGGTAGCA | — | 518 |
| MKW0163 | gatttagactaccccaaaaacgaaggggactaaaacTGCTACCAGCTCAACCATAACATGACCA | — | 519 |
| MKW0164 | gatttagactaccccaaaaacgaaggggactaaaacCGAACTGCACCTCATGGTCATGTTATGG | — | 520 |
| MKW0165 | gatttagactaccccaaaaacgaaggggactaaaacCCATAACATGACCATGAGGTGCAGTTCG | — | 521 |
| crRNA N1-1 | gatttagactaccccaaaaacgaaggggactaaaacGCACCCCGCATTACGTTTGGTGGACCCT | X | 522 |
| crRNA N1-2 | gatttagactaccccaaaaacgaaggggactaaaacAGGGTCCACCAAACGTAATGCGGGGTGC | X | 523 |
| crRNA N2-1 | gatttagactaccccaaaaacgaaggggactaaaacTGCACAATTTGCCCCCAGCGCTTCAGCG | X | 524 |
| crRNA N2-2 | gatttagactaccccaaaaacgaaggggactaaaacCGCTGAAGCGCTGGGGGCAAATTGTGCA | X | 525 |
| crRNA N3-1 | gatttagactaccccaaaaacgaaggggactaaaacATCACATTGGCACCCGCAATCCTGCTAA | — | 526 |
| crRNA N3-1 | gatttagactaccccaaaaacgaaggggactaaaacTTAGCAGGATTGCGGGTGCCAATGTGAT | — | 527 |

Certain guides were tested in combination with LAMP primer sets. Each guide tested was ranked as either 4: No detection; 3: Poor detection; 2: Good detection; or 1: Best detection. The results of the guide screening are demonstrated in Table 24.

TABLE 24

| target/primer | | | | | | | | | |
|---|---|---|---|---|---|---|---|---|---|
| sets | crRNA | 3000 copies | | 30 copies | | NTC | | S/N | RANK |
| B3 (orf1ab) | XLcr-507 | 381560 | 606255 | 751128 | 479722 | 11912 | 11033 | 53.6435 | 1 |
| B3 (orf1ab) | XLcr-506 | 159355 | 242958 | 362670 | 283619 | 12702 | 12853 | 25.2901 | 2 |
| B3 (orf1ab) | XLcr-505 | 84626 | 27520 | 13134 | 12996 | 11661 | 9565 | 1.23104 | 4 |
| B5 (orf1ab) | XLcr-509 | 596771 | 655653 | 626136 | 700660 | 11049 | 11547 | 58.7182 | 1 |
| B5 (orf1ab) | XLcr-510 | 500593 | 583394 | 624045 | 612371 | 13989 | 13380 | 45.1758 | 2 |
| B5 (orf1ab) | XLcr-512 | 38997 | 50342 | 79652 | 133126 | 12817 | 12506 | 8.40256 | 3 |
| CN-4B7 (N) | MKW0307 | 193718 | 158289 | 163775 | 172492 | 5365 | 5149 | 31.9828 | 2 |
| B5 (orf1ab) | XLcr-511 | 12617 | 15145 | 19248 | 27464 | 11217 | 11329 | 2.07185 | 4 |
| HK5 (orf1ab) | XLcr-530 | 808713 | 702139 | 727598 | 727101 | 8336 | 8819 | 84.7974 | 1 |
| HK5 (orf1ab) | XLcr-531 | 743506 | 735770 | 671386 | 575016 | 9777 | 8956 | 66.5351 | 2 |
| HK5 (orf1ab) | XLcr-529 | 1019348 | 581204 | 750566 | 660271 | 16473 | 16749 | 42.4669 | 2 |
| HK7-B7 | XLcr-534 | 788340 | 744356 | 666083 | 695888 | 12366 | 11219 | 57.7473 | 2 |
| (orf1ab) | | | | | | | | | |
| HK7-F7 | XLcr-535 | 866522 | 757465 | 678074 | 730362 | 9309 | 8150 | 80.6711 | 1 |
| (orf1ab) | | | | | | | | | |
| CN-4B7 (N) | MKW0306 | 358246 | 318146 | 278357 | 29721 | 5229 | 5164 | 29.6428 | 2 |
| HK7-F7 | XLcr-536 | 949197 | 885495 | 786778 | 740809 | 10180 | 9335 | 78.2776 | 1 |
| (orf1ab) | | | | | | | | | |
| CN-4B7 (N) | MKW0304 | 117477 | 132281 | 132603 | 126557 | 5861 | 5830 | 22.1675 | 2 |
| HK7-F7 | XLcr-534 | 960662 | 817735 | 779298 | 689392 | 13810 | 12804 | 55.1849 | 2 |
| (orf1ab) | | | | | | | | | |
| HK7-F7 | XLcr-537 | 864567 | 752316 | 755534 | 679622 | 18715 | 19172 | 37.8799 | 2 |
| (orf1ab) | | | | | | | | | |
| CN-4B7 (N) | MKW0303 | 112354 | 102155 | 71727 | 93316 | 5624 | 5472 | 14.8741 | 3 |
| CN-4F7 (N) | MKW0304 | 235526 | 220675 | 237769 | 251256 | 5856 | 5720 | 42.2447 | 2 |
| CN-4F7 (N) | MKW0306 | 228295 | 211651 | 132373 | 166404 | 5417 | 5155 | 28.2612 | 2 |
| CN-4F7 (N) | MKW0303 | 76253 | 57912 | 66575 | 71504 | 5846 | 5543 | 12.1239 | 3 |
| CN-4F7 (N) | MKW0307 | 244513 | 239678 | 238032 | 293421 | 73770 | 5403 | 6.71255 | 3 |
| JP13 (orf1ab) | MKW0162 | 776366 | 681657 | 9653 | 667011 | 9754 | 9637 | 34.8958 | 2 |
| JP13 (orf1ab) | MKW0160 | 804053 | 860405 | 16386 | 686270 | 13490 | 12905 | 26.6208 | 2 |
| JP2 (orf1ab) | MKW0163 | 785006 | 669887 | 749394 | 698177 | 9265 | 9736 | 76.1839 | 1 |
| CN-5B7 (N) | MKW0303 | 54150 | 42947 | 42340 | 45260 | 5195 | 33100 | 2.2875 | 4 |
| JP2 (orf1ab) | MKW0162 | 900163 | 796992 | 807423 | 792384 | 15817 | 13983 | 53.6848 | 2 |
| CN-5B7 (N) | MKW0306 | 356470 | 342436 | 317549 | 322836 | 5349 | 322337 | 1.95426 | 4 |
| CN-5F7 (N) | MKW0306 | 322802 | 302853 | 294700 | 305699 | 5273 | 5060 | 58.105 | 2 |
| CN-5F7 (N) | MKW0303 | 24608 | 22195 | 25378 | 20977 | 5448 | 5339 | 4.2973 | 3 |
| JP2 (orf1ab) | MKW0160 | 615770 | 445162 | 405453 | 660643 | 12836 | 14247 | 39.364 | 2 |
| N2-3 (N) | MKW0301 | 139665 | 86572 | 87309 | 67359 | 5199 | 59696 | 2.38337 | 4 |
| N2-3 (N) | MKW0300 | 194311 | 185570 | 154457 | 170884 | 5926 | 145851 | 2.14355 | 4 |
| N2-8 (N) | MKW0300 | 309406 | 218624 | 176785 | 232912 | 6234 | 6268 | 32.7705 | 2 |
| JP2 (orf1ab) | MKW0161 | 788645 | 713904 | 702131 | 704646 | 35679 | 35324 | 19.8129 | 3 |
| N2-8 (N) | MKW0301 | 122293 | 91069 | 91177 | 88135 | 5602 | 5495 | 16.1593 | 3 |
| N2-8 (N) | MKW0302 | 80342 | 39161 | 49717 | 42488 | 5640 | 5411 | 8.34392 | 3 |
| N2-8 (N) | MKW0299 | 8398 | 7396 | 7883 | 6907 | 4111 | 4682 | 1.68202 | 4 |
| JP5 (orf1ab) | MKW0165 | 696060 | 682256 | 15141 | 719209 | 13834 | 15635 | 24.9194 | 2 |
| N2-8 (N) | MKW0298 | 14876 | 8856 | 7590 | 3456 | 3843 | 4707 | 1.29193 | 4 |
| N2-9 (N) | MKW0300 | 320588 | 267128 | 270173 | 273597 | 6644 | 7594 | 38.1927 | 2 |
| N2-9 (N) | MKW0301 | 205854 | 162351 | 148890 | 198734 | 5822 | 5303 | 31.2498 | 2 |
| JP5 (orf1ab) | MKW0164 | 63738 | 55591 | 11900 | 52679 | 12010 | 11523 | 2.74419 | 4 |
| N2-9 (N) | MKW0299 | 6750 | 6278 | 6013 | 6067 | 5151 | 5076 | 1.18119 | 4 |
| N2-9 (N) | MKW0298 | 5850 | 5314 | 5016 | 5075 | 4809 | 4695 | 1.06176 | 4 |

This screening demonstrated the empirical identification of unique sets of LAMP primers and guide polynucleotides for detecting the presence of SARS-CoV-2.

### Example 7: Cross Reactivity Study

This example demonstrates that methods described herein are sensitive and specific. Specifically, the present example demonstrates that the methods described herein do not result in false positive detection of SARS-CoV-2 due to cross reactivity.

Cross-Reactivity Pools: The cross-reactivity panel were tested in five pools, each consisting of two organisms. To create the two panel-member pools, the stock concentration of each organism was diluted in nuclease-free water following the scheme in worksheet “Cross Reactivity Calculations.” The final concentration for each organism within the pool will be 2×10⁴ genome equivalents/μL (for bacteria and yeast) or 2×10³ genome equivalents/μL (for viruses), for a final assay concentration of 10⁶ genome equivalents/mL of VTM for bacteria and yeast, or 10⁵ genome equivalents/mL of VTM for viruses. Pools may be prepared in advance of the study and stored at a temperature at or below negative 70° C.

Samples: Each sample tested in this study was created by the addition of 10 microliters of the pooled, diluted organism stock (described above) to 200 microliters of lysis-treated negative matrix (e.g. 200 microliters of the NM AFTER the addition of 225 microliters of the PureLink™ lysis buffer and Proteinase K, and incubation at 56° C. for 15 minutes). This contrived sample was then extracted using the PureLink™ Viral DNA/RNA Mini Kit, following the manufacturer's instructions with a final elution volume of 30 microliters. Eight microliters of this eluted sample was used as template for each of the two SARS-CoV-2 analytes targeted by the Sherlock™ CRISPR SARS-CoV-2 kit (i.e., ORF1ab and N target analytes, and the RNaseP control). Three replicate aliquots of each eluted sample will be tested.

Controls: i. Extraction Control: RNaseP detection serves as an extraction control in the absence of a SARS-CoV-2 signal. ii. No Template Control: A “no input RNA” reaction is set up as a negative control for amplification and to determine background fluorescence levels in the Cas detection reaction. A negative control was performed for each LAMP primer set and each guide to be tested. The negative control was created by replacing the eight microliter template volume in the LAMP reaction with an equal volume of nuclease-free water. iii. Positive Control: A positive control for amplification and detection of the SARS-CoV-2 target analytes will be performed for each ORF1ab and N LAMP primer set and each ORF1ab and N guide to be tested. The positive control is created by replacing the eight microliter template volume in the LAMP reactions with an equal volume of viral genomic RNA extracted from cultured SARS-CoV-2 virus propagated in Vero cells, stabilized in Trizol and transported to Sherlock Biosciences. This viral stock was quantified by digital PCR and diluted to a concentration of 4800 copies per microliter in nuclease free water, aliquoted for single use.

The cross reactivity of each target primer and guide set was independently determined under this protocol. Five organism pools were created and used to perform the in vitro cross-reactivity study. Each organism pool consisted of nucleic acid from two organisms. Samples will be created by spiking SARS-CoV-2 Negative Matrix with quantified, pooled stocks of extracted nucleic acids from two organisms at clinically relevant concentrations. Three replicate aliquots from each organism pool were tested using the Sherlock™ CRISPR SARS-CoV-2 kit.

Organism pool creation: Quantified organism stock pools described in Table 25 below were prepared Ten microliters of each pooled, quantified organism stock was spiked into 200 microliters of negative matrix after addition of 225 microliters of the PureLink™ lysis buffer/Proteinase K, and incubation at 56° C. for 15 minutes.

TABLE 25
Organism Stock Pool Concentrations Organism Pool

| | | | Genome | Genome |
|---|---|---|---|---|
| | | | copies/ul | copies/mL |
| | | | pooled | contrived |
| | Organism | Source | organism stock | clinical sample |
| 1 | Human | ATCC ® VR-740D | 2.0 × 10³ | 1.0 × 10⁵ |
| | coronavirus 229E | | | |
| | Human | ATCC ® VR-1558D | 2.0 × 10³ | 1.0 × 10⁵ |
| | coronavirus OC43 | | | |
| 2 | Human | ATCC ® VR-3262SD | 2.0 × 10³ | 1.0 × 10⁵ |
| | coronavirus HKU1 | | | |
| | Human | ATCC-3263SD | 2.0 × 10³ | 1.0 × 10⁵ |
| | coronavirus NL63 | | | |
| 3 | Influenza A | VR-95DQ | 2.0 × 10³ | 1.0 × 10⁵ |
| | Influenza B | VR-1885DQ | 2.0 × 10³ | 1.0 × 10⁵ |
| 4 | Respiratory | ATCC ® VR-1580DQ | 2.0 × 10³ | 1.0 × 10⁵ |
| | syncytial virus | | | |
| | Pseudomonas | ATCC ® 27853D-5 | 2.0 × 10⁴ | 1.0 × 10⁶ |
| | aeruginosa | | | |
| 5 | Staphylococcus | ATCC ® 12228D-5 | 2.0 × 10⁴ | 1.0 × 10⁶ |
| | epidermis | | | |
| | Candida albicans | ATCC ® 10231D-5 | 2.0 × 10⁴ | 1.0 × 10⁶ |

LAMP reactions were performed. For each extracted sample, one LAMP reaction was performed for each of three primer sets. Additionally, a positive control for detection of SARS-CoV-2 targets was included as described (consisting of previously extracted viral RNA at 4800 cp/μL). One negative control (consisting of nuclease-free water instead of template, as described) was performed for each of the three LAMP Primer Set and Cas reactions.

Interpretation of test sample results: 1. Target (N, Orf1ab, RNaseP) interpretation: A sample was considered positive for a target if the Cas signal increased ≥5-fold at the T10 reading over a valid Negative Control (“no RNA added”) for that target. SARS-CoV-2 (COVID-19) Positive Result interpretation: A sample was positive for COVID-19 if at T10, a contrived sample's fluorescent Cas signal is ≥5-fold at the T10 reading over a valid Negative control's fluorescent Cas signal for one or more of SARS-CoV-2 target analytes (i.e., N or ORF1ab). SARS-CoV-2 (COVID-19) Negative Result interpretations: A sample was negative for COVID-19 if at T10: a. a contrived sample's fluorescent signal was less than 5-fold greater than a valid Negative Control signal for both SARS-CoV-2 target analytes b. AND the RNaseP signal was positive (the RNaseP fluorescent signal is at least 5-fold greater than a valid Negative Control signal at the T10 reading). 4. Invalid Results interpretation: A specimen was invalid if at the T10 reading: a. a contrived sample's fluorescent signal is less than 5-fold greater than a valid Negative Control signal for both SARS-CoV-2 (N and ORF1ab) target analytes at the T10 reading b. AND the RNaseP signal was less than 5-fold greater than a valid Negative Control signal at the T10 reading. Any sample with an invalid test result may be retested starting at the extraction step.

Statistical/Analysis Methods, Sample Size and Acceptance Criteria: If 0/3 replicates for an organism pool were positive for both SARS-CoV-2 targets, all organisms in that pool were said to show no cross-reactivity with the Sherlock™ CRISPR SARS-CoV-2 kit. a. Invalid samples were excluded from the result analysis and retested. b. In the event that a positive signal for one or more SARS-CoV-2 targets was detected for any replicate of a pool, each organism from that pool was tested individually with three (n=3) replicates following the protocol outlined above for the testing of organism pools. i. Organisms that show 0/3 replicates with a positive detection for both SARS-CoV-2 target analytes were said to show no cross-reactivity with the Sherlock™ CRISPR SARS-CoV-2 kit. ii. Organisms that show N=1 to 3 replicates with a positive detection for either SARS-CoV-2 target were said to potentially cross-react with the Sherlock™ CRISPR SARS-CoV-2 kit. Serial dilutions of the “reactive” organism may be tested in triplicate until 0/3 replicates are negative for SARS-CoV-2 detection.

Wet testing against high risk pathogenic organisms of the respiratory tract selected based on disease prevalence, disease risk, homology to assay specific targets and homology to SARS-CoV-2 genome was performed to confirm the results of in silico analysis. Each organism identified below was tested in triplicate with the Sherlock™ CRISPR SARS-CoV-2 kit by spiking diluted organism stock into lysis-treated pooled nasopharyngeal swab matrix. All replicates were negative for SARS-CoV-2.

| Organism | ATCC Cat. Number | Concentration | ORF1ab | N | RNaseP |
|---|---|---|---|---|---|
| Human coronavirus 229E | ATCC ® VR-740D | 1 × 10⁵ copies/mL | 0/3 | 0/3 | 3/3 |
| Human coronavirus OC43 | ATCC ® VR-1558D | 1 × 10⁵ copies/mL | 0/3 | 0/3 | 3/3 |
| Human coronavirus HKU1 | ATCC ® VR-3262SD | 1 × 10⁵ copies/mL | 0/3 | 0/3 | 3/3 |
| Human coronavirus NL63 | ATCC ® 3263SD | 1 × 10⁵ copies/mL | 0/3 | 0/3 | 3/3 |
| Influenza A | VR-95DQ | 1 × 10⁵ copies/mL | 0/3 | 0/3 | 3/3 |
| Influenza B | VR-1885DQ | 1 × 10⁵ copies/mL | 0/3 | 0/3 | 3/3 |
| Respiratory syncytial virus | ATCC ® VR-1580DQ | 1 × 10⁵ copies/mL | 0/3 | 0/3 | 3/3 |
| Pseudomonas aeruginosa | ATCC ® 27853D-5 | 1 × 10⁶ copies/mL | 0/3 | 0/3 | 3/3 |
| Staphylococcus epidermis | ATCC ® 12228D-5 | 1 × 10⁶ copies/mL | 0/3 | 0/3 | 3/3 |
| Candida albicans | ATCC ® 10231D-5 | 1 × 10⁶ copies/mL | 0/3 | 0/3 | 3/3 |

### Example 8: Limit of Detection Testing for Pooled Saliva Samples

The present example describes tests for determination of the limit of detection of the SARS-CoV-2 diagnostic described herein using saliva samples.

To test limits of detection of saliva using diagnostic methods described herein, pooled human saliva samples were added 1:1 to Zymo DNA/RNA Saliva Kit (R1210-1). 200 μl of the pooled saliva was then spiked with SARS-CoV-2 positive control (SeraCare 0505-129). RNA was then extracted from the positive control spiked saliva samples using Purelink Extraction Kit as described herein and eluted in 30 ul. Table 26 demonstrates sensitive detection of SARS-CoV-2 extracted from saliva.

TABLE 26

| Saliva/Zymo Preservative | | | |
|---|---|---|---|
| Sample Concentration | N | Orf | Sherlock Positive |
| 12 | 3/3 | 3/3 | 3/3 |
| 4 | 3/3 | 3/3 | 3/3 |
| 2 | 3/3 | 3/3 | 3/3 |
| 1.5 | 5/6 | 6/6 | 6/6 |
| 1 | 3/3 | 3/3 | 3/3 |
| 0.75 | 6/6 | 4/6 | 6/6 |
| 0.375 | 1/3 | 2/3 | 2/3 |
| 0.1875 | 0/3 | 1/3 | 1/3 |
| 0 | 0/7 | 0/7 | 0/7 |

To further demonstrate the capability of the assay described herein to detect SARS-CoV-2 in saliva the diagnostic methods and compositions described herein were tested on saliva without RNA extraction. Pooled saliva spiked with SARS-CoV-2 positive control was either mixed 1:1 with Quick Extract DNA Buffer (15 ul Quick Extract DNA Buffer to 15 ul of Saliva+SeraCare positive control) heated at 65° C. for 6 min, heated at 98° C. 3 min, and cooled to 4° C. or 3 ul of Proteinase K and 12 ul of H20 was added to 15 ul of Saliva+Preservative+Seracare then heated at 55° C. for 15 min, 98° C. for 3 min, and cooled to 4° C. Tables 27 and 28 demonstrate sensitive detection of SARS-CoV-2 extracted from saliva.

TABLE 27

| | |
|---|---|
| N | Orf |

Quick Extract DNA

| | | | | | |
|---|---|---|---|---|---|
| 50 | cp/ul | 200 | cp/rxn | 3/3 | 2/3 |
| 25 | cp/ul | 100 | cp/rxn | 2/3 | 3/3 |
| 5 | cp/ul | 20 | cp/rxn | 0/3 | 1/3 |

| | | | |
|---|---|---|---|
| 0 | 0 | 0/1 | 0/1 |

Heating + Proteinase K

| | | | | | |
|---|---|---|---|---|---|
| 50 | cp/ul | 200 | cp/rxn | 3/3 | 1/3 |
| 25 | cp/ul | 100 | cp/rxn | 3/3 | 1/3 |
| 5 | cp/ul | 20 | cp/rxn | 2/3 | 1/3 |

| | | | |
|---|---|---|---|
| 0 | 0 | 0/1 | 0/1 |

Heating Only

| | | | | | |
|---|---|---|---|---|---|
| 50 | cp/ul | 400 | cp/rxn | 3/3 | 1/3 |
| 25 | cp/ul | 200 | cp/rxn | 3/3 | 0/3 |
| 5 | cp/ul | 40 | cp/rxn | 0/3 | 0/3 |

| | | | |
|---|---|---|---|
| 0 | 0 | 0/1 | 0/1 |

TABLE 28

Sherlock Positive

| Sample | Quick | |
|---|---|---|
| Concentration | Extract | Heating + |
| copies/ul | DNA | Proteinase K |
| 50 | 3/3 | 3/3 |
| 25 | 3/3 | 3/3 |
| 5 | 1/3 | 2/3 |
| 0 | 0/1 | 0/1 |

### Example 9: Automated Workflow

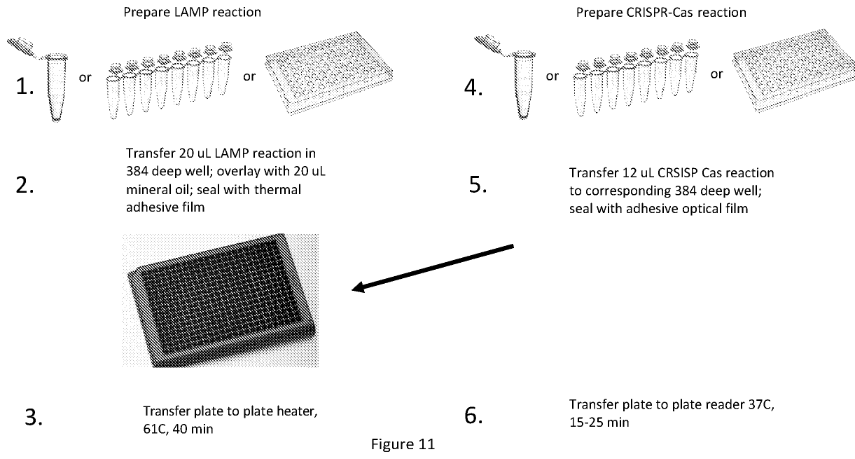

The present example demonstrates, as described herein, that steps of the diagnostic assay of the present disclosure can be combined to improve speed, accuracy and ease of workflow. FIG. 11 demonstrates an efficient workflow in which certain steps are combined and performed sequentially in a single vessel. In this exemplary workflow an amplification reaction (e.g., LAMP) is prepared (1), then aliquoted to a 384 well plate (2). Following the amplification reaction (3) a CRISPR/Cas collateral activity assay is prepared (4) and aliquoted to the same 384 well plate for activation of CRISPR/Cas collateral activity (5) and detection of associated signal (6).

Each sample analyzed in the automated process disclosed herein was plated in duplicate in a 384 well plate. 7 μL of lysis solution (e.g., proteinase K or Quick Extract) was added to each well of the 384 well plate. Subsequently, 7 μL of sample was added to each well of the 384 well plate. The plate was incubated at 55° C. for 15 min followed by a 3 minute incubation at 98° C. 8 μL of the LAMP amplification reagent was added to each well. One of the two duplicate samples received SARS-Cov-2 LAMP amplification reagent and the remaining duplicate received the control LAMP amplification reagent. 20 μL of mineral oil was added to each well of the 384 well plate. The plate was incubated at 61° C. for 40 minutes. 5 μL of SARS-CoV-2 Cas detection reagent (see “Target CRISPR Cas Master Mix Recipe”) was added to SARS-CoV-2 target containing wells and 5 μL of control Cas detection reagent was added to control target containing wells. Signal detection was completed on a fluorescent plate reader at 37° C. with excitation-emission of 485 and 528 nanometers, respectively. Notably, the plate was not cooled to 4° C., but room temperature after the LAMP reaction.

Target CRISPR Cas Master Mix Recipe

| | | |
|---|---|---|
| Reagent | Volume per Reaction | Volume Total |

| | | | |
|---|---|---|---|
| Premix | 7.5 | μL | 7.5 μL × (12 + 1)= |
| crRNA N | 2.25 | uL | 2.25 uL × (12 + 1)= |
| crRNA O | 2.25 | μL | 2.25 μL × (12 + 1)= |
| MgCl2 | 0.23 | μL | 0.23 μL × (12 + 1)= |
| Total Volume | 12.23 | μL | 12.23 μL × (12 + 1)= |

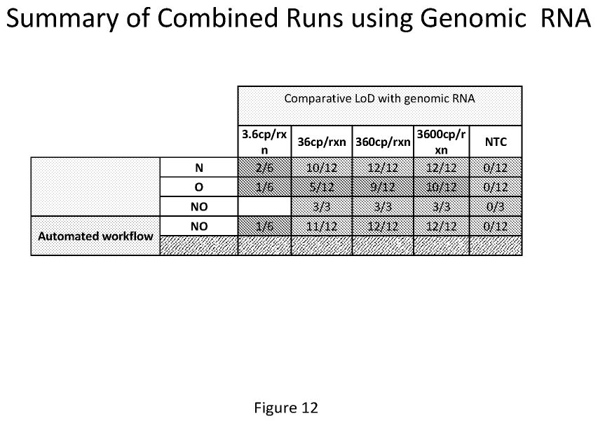

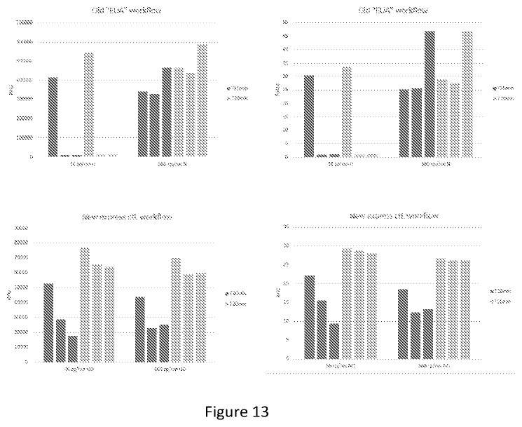

FIG. 12 demonstrates that combining performing the amplification, CRISPR/cas activation and detection on a single plate results in a simpler workflow as well as reliable results. Notably, combining a cRNA detecting N and a cRNA detecting ORF1ab in a single detection reaction results in sensitive detection of SARS-CoV-2. FIG. 13 demonstrates significant differences between RFUs determined 10 and 20 minutes after detection is initiated in the combined workflow which is not observed otherwise.

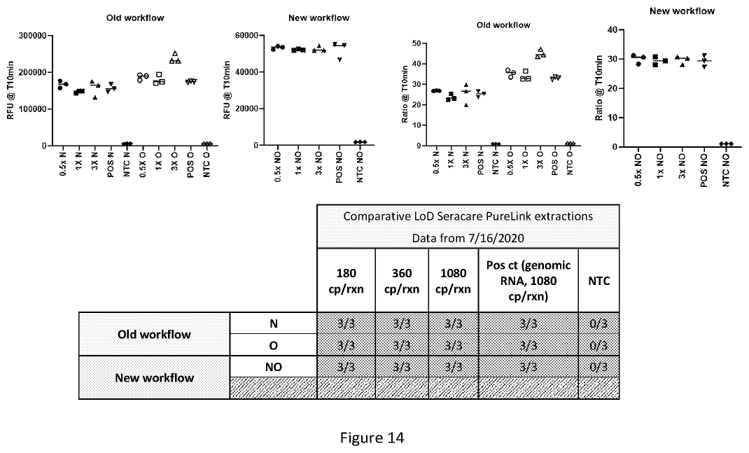

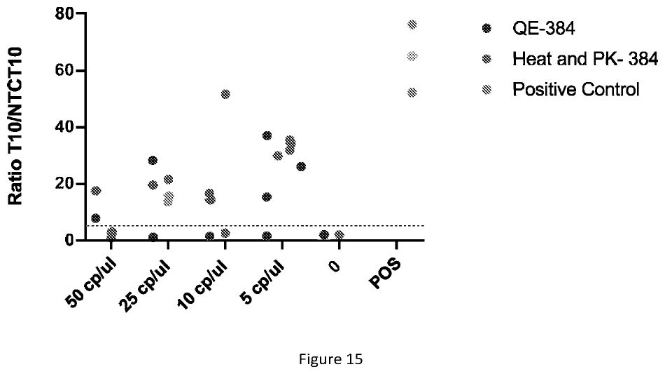

Additionally, FIG. 14 shows a comparison of SARS-CoV-2 containing saliva samples extracted using methods described herein and assayed using the combined workflow (“new workflow”). FIG. 15, shows further confirmation of the sensitivity of the methods described herein. Saliva samples (10 μL of pooled saliva at 50, 25, 10, 5 and 0 copies/μL sample) were assayed using the 384 well plate workflow described herein. Briefly proteinase K (PK) or Quick Extract (QE) we added to the sample and heated at 65° C. for 6 min and 98° C. for 3 min. Then a LAMP master mix was added at heated at 61° C. for 40 min. A Cas master mix was then added and the plate was incubated on a plate reader at 37° C. while signal was detected. Notably the combined workflow provides sensitive detection of SARS-CoV-2.

### Example 10: SARS-CoV-2 Detection from Patient Nasopharyngeal Swabs

The present example further demonstrates, as described herein, the sensitivity and specificity of the SHERLOCK CRISPR SARS-CoV-2 kit. The present example describes detection of SARS-CoV-2 from a total of 20 COVID-19 patient samples (10 positive and 10 negative) from nasopharyngeal swabs. Selected COVID-19 patient samples were tested on previously validated RT-qPCR assays (CDC, Abbott, m2000). Positive samples were selected based on a broad range of cycle threshold (Ct) values, comprising an average of low (μ=7.11), mid (μ=17.2), and high (μ=27.9) Ct values. Nucleic acids extraction from nasopharyngeal swab patient samples were performed using EZ1 Advanced system (Qiagen). Following the SHERLOCK CRISPR SARS-CoV-2 kit instructions, the extracted material was subjected to reverse transcriptase loop-mediated amplification. Amplified products were incubated with Cas13a enzyme complexed with CRISPR guide RNAs specific to SARS-CoV-2 targets. Fluorescent read outs of the cleaved reporter molecules were taken at 2.5 minute intervals for a total of 10 minutes on a microplate reader (BioTek). Data output of relative fluorescent unit ratios were normalized to a no-template control. All 20 COVID-19 patient samples were correctly diagnosed with up to 100% accuracy. All controls, including RNase P, showed expected findings with 5500 copies/μl detected for diluted positive control isolate (BEI). For COVID-19 positive samples, normalized ratios ranged from 16.45-49.17 and 33.82-48.15 for N and ORF1ab gene targets, respectively. Fluorescence ratios on negative samples ranged from 0.54-1.28 and 0.84-4.93 for N and ORF1ab gene targets, respectively. Determined ratios were sufficiently greater or less than the pre-established 5-fold change in fluorescence read output, obviating interpretation of any borderline results.

### Example 11: Real Time Multiplexed SARS-CoV-2 Detection

The present example confirms that thermostable Cas enzymes as described herein permit multiple reaction steps to be performed in a single reaction vessel (e.g., “one pot”). Use of thermostable Cas reduces or eliminates certain processing and/or transfer steps. The present example demonstrates that with use of thermostable Cas all reaction steps beyond nucleic acid isolation may be performed in a single vessel.

The present example demonstrates that use of a new thermostable Cas12 protein described herein (SLK-9 (also referred to as rs9, interchangeably; SEQ ID NO: 15) that is compatible with LAMP provides an improved Real Time SHERLOCK system (RT-SHERLOCK) that dramatically simplified the workflow from a two-step workflow to a single reaction, meanwhile providing real time signal readout. Furthermore, combination of two different CRISPR-Cas systems (SLK-9; SEQ ID NO:15 and AacCas12b; SEQ ID NO: 3) generated the first real time multiplexed CRISPR based diagnostic platform (Duplex Aac/rs9-cas12 Real Time Sherlock; DARTS) that is capable of detecting SARS-CoV-2 RNA and human RnaseP internal control simultaneously.

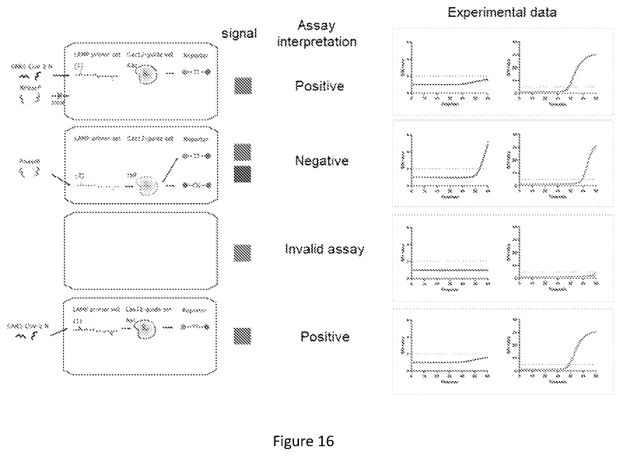

The one step workflow of RT-SHERLOCK and DARTS is performed by adding extracted or unextracted COVID-19 patient anterior nasal swab or saliva samples into a reaction tube containing RT-SHERLOCK or DARTS reaction mix followed by monitoring fluorescence signal change at real time. Extracted samples were purified by Purelink extraction kit according to its protocol and eluted into water. Unextracted samples were simply heat lysed with addition of Proteinase K and RNAsecure (65 C 15 min, 95 C 10 min). An exemplary DARTS design (DARTSv1) is shown in Table 29 wherein DARTSv1 uses AacCas12b system to detect N gene and rs9 system to detect Rnase P (RP) internal control. FIG. 16 shows experimental data demonstrating the ability of the duplexed system to detect both SARS-CoV-2 and RP simultaneously.

TABLE 29

| DARTS design | Aac Cas12b | Rs9 cas12a |
|---|---|---|
| Target | SARS-CoV-2 N gene | RnaseP internal control |
| Reporter | polyT7-FAM | polyT7-FAM |
| | | polyC7-TexasRed |
| Fluorescence channel | x1-m1 | x1-m1 |
| | | x4-m4 |
| Temperature | 56 C. | |
| Guide RNA | Guide 1 (XL-A226) | Guide 2 (XL-374) |
| Primer set | CNFB | RP |

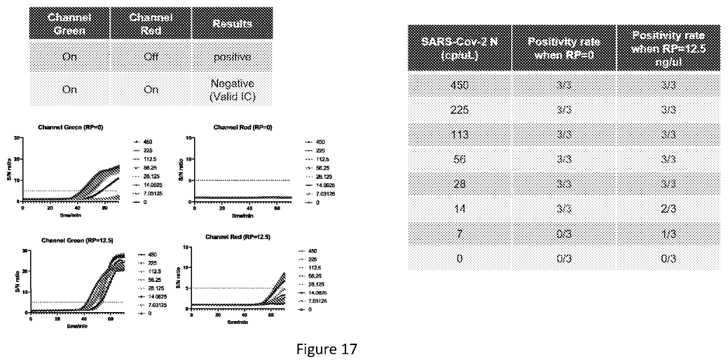

Initial evaluation of the limit of detection of DARTSv1 demonstrated that the LOD is 14-28 cp/μl (FIG. 17).

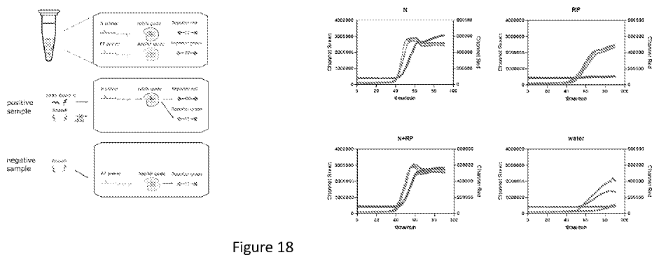

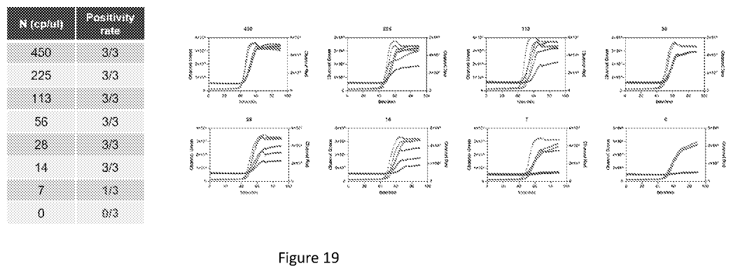

A further exemplary DARTS platform (DARTs v2) contained RT-LAMP reaction mix to provide sufficient reagent for duplexed LAMP amplification, two LAMP primer sets for N and RP, SLK9 enzymes with crRNA targeting N, AacCas enzyme with crRNA targeting RP, FAM-quencher modified T reporter, and HEX-quencher modified C reporter (FIGS. 18 and 19). DARTS is the first demonstration of a multiplexed real-time CRIPSR diagnostic platform. To use the DARTS assay, the only operational step by the user is to add samples into DARTS reaction and put the reaction into a device with fluorescence monitor and temperature control such as plate readers or qPCR instruments. The exemplary DARTS assay was conducted at 56° C., which is lower than optimal SLK9 reaction temperature to comprise for the weaker thermal stability of Aac system. In the reaction, when samples were added into DARTS, N or RP were amplified by corresponding LAMP primer sets, followed by the activation of corresponding Cas enzymes. When SLK9 is activated, it will cleave both C and T reporter, lighting up both FAM and HEX fluorescence. When Aac was activated, it only cleaved T reporter, lighting up only FAM fluorescence. The assay result interpretation follows simple and intuitive “two-line means positive, one-line means negative” rule: positive samples show ON signal in both FAM and HEX channel, negative samples show ON signal in FAM channel and OFF signal in HEX channel. Assays were determined as invalid if OFF signal are seen in FAM channel or both channels. The LOD for DARTSv2 was found to be 7-14 cp/μl (FIG. 19).

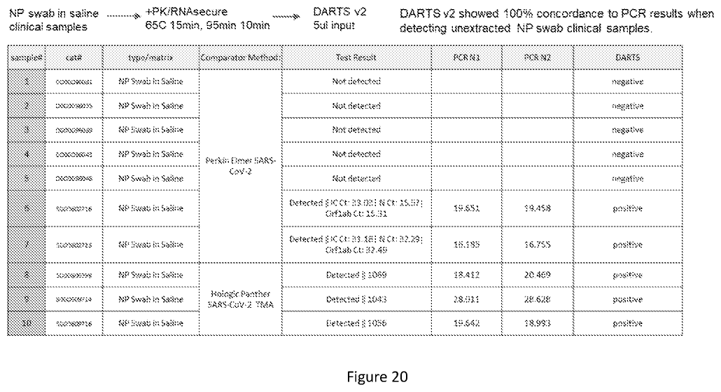

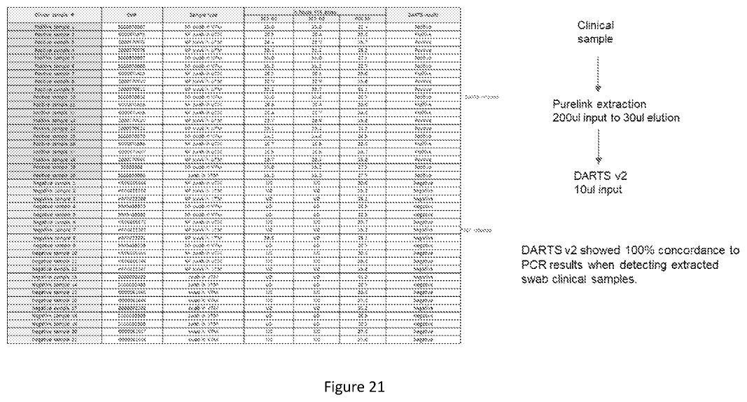

The RT-SHERLOCK and DARTS assays were evaluated on a combined total of 60 positive and negative patient samples with or without extraction, and achieved a 98% concordance to traditional RT-PCR (58 correctly identified out of 60 total; FIGS. 20 and 21). No false-positives were observed. The time-to-result can be as fast as 12 minutes depending on the patient samples and the utilized extraction methods. The RT-SHERLOCK analytical limits of detection are 0.5 copies/uL for extracted samples and 10-20 copies/μL for unextracted samples depending on sample type. The DARTS analytical limits of detection are 10 copies/uL for extracted samples and 60 copies/μL for unextracted samples.

Exemplary DARTS detection of clinical sample was performed by adding 10 μL or 5 μL pretreated clinical sample directly into a DARTS reaction mix and then measured on a QuantStudio 5 qPCR instrument for florescence readout at 56° C. An exemplary DARTS reaction mix is shown in Table 11-1.

TABLE 11-1

| | | | Amount per reaction (uL) | |
|---|---|---|---|---|
| | Stock | | Extracted | Unextracted |
| DARTS reaction mix | concentration | | sample | sample |
| Warmstart LAMP reaction | 2x | | 25 | 25 |
| mix (NEB) | | | | |
| CNFB primer mix | — | | 2.2 | 2.2 |
| RPFB primer mix | — | | 2.2 | 2.2 |
| Aac Cas12b enzyme | 2 | mg/mL | 1.2 | 1.2 |
| SLK9 Cas12a enzyme | 2.83 | mg/mL | 0.65 | 0.65 |
| AacCas12b crRNA for RP | 10 | uM | 2.6 | 2.6 |
| SLK9 Cas12a crRNA for N | 10 | uM | 0.6 | 0.6 |
| Reporter C | 20 | uM | 0.5 | 0.5 |
| Reporter T | 20 | uM | 0.5 | 0.5 |

| | | | |
|---|---|---|---|
| Water | — | 4.55 | 9.55 |
| Sample | — | 10 | 5 |
| Total volume | — | 50 | 50 |

An exemplary primer mix is shown in Table 11-2.

TABLE 11-2

| Primer mix | Stock concentration | Amount (uL) |
|---|---|---|
| F3 | 100 uM | 10 |
| B3 | 100 uM | 10 |
| FIP | 100 uM | 80 |
| BIP | 100 uM | 80 |
| LF | 100 uM | 20 |
| LB | 100 uM | 20 |
| Total volume | — | 220 |

Exemplary sequences for a DARTS reaction are shown in Table 11-3.

TABLE 11-3

| Name | Sequence |
|---|---|
| Aac Cas12b | GTCTAGAGGACAGAATTTTTCAACGGGTGTGCCAAT |
| crRNA for | GGCCACTTTCCAGGTGGCAAAGCCCGTTGAGCTTCT |
| RP | CAAATCTGAGAAGTGGCACAGTGGAGGAGTGTCTTT |
| | TCAA (SEQ ID NO: 742) |
| SLK9 Cas12a | AAUUUCUACUAUUGUAGAUCUCCUGCUAGAAUGGCU |
| crRNA for N | GGCAAUGGC (SEQ ID NO: 743) |
| CNFB | GCTTCTACGCAGAAGGGA (SEQ ID NO: 744) |
| primer: F3 | |
| CNFB | GTGACAGTTTGGCCTTGT (SEQ ID NO: 745) |
| primer: B3 | |
| CNFB | CTACTGCTGCCTGGAGTTGAATTCCTCTTCTCGTTC |
| primer: FIP | CTCATC (SEQ ID NO: 746) |
| CNFB | GCTTTGCTGCTGCTTGACAGTGTTGTTGGCCTTTAC |
| primer: BIP | CA (SEQ ID NO: 747) |
| CNFB | CTTGAACTGTTGCGACTACGT (SEQ ID |
| primer: LF | NO: 748) |
| CNFB | ATTGAACCAGCTTGAGAGCAAA (SEQ ID |
| primer: LB | NO: 749) |
| RPFB primer: | TTGATGAGCTGGAGCCA (SEQ ID NO: 750) |
| F3 | |
| RPFB primer: | CACCCTCAATGCAGAGTC (SEQ ID NO: 751) |
| B3 | |
| RPFB primer: | GTGTGACCCTGAAGACTCGGTTTTAGCCACTGACTC |
| FIP | GGATC (SEQ ID NO: 752) |
| RPFB primer: | CCTCCGTGATATGGCTCTTCGTTTTTTTCTTACATG |
| BIP | GCTCTGGTC (SEQ ID NO: 753) |
| RPFB primer: | ATGTGGATGGCTGAGTTGTT (SEQ ID |
| LF | NO: 754) |
| RPFB primer: | GGCATGCTGAGTACTGGACCTC (SEQ ID |
| LB | NO: 755) |
| Reporter C | /5TexRd-XN/CCCCCCC/3IAbRQSp/ (SEQ ID |
| | NO: 756) |
| Reporter T | /56-FAM/TTTTTTT/3IABkFQ/ (SEQ ID |
| | NO: 757) |

RT-SHERLOCK and DARTS assays based on a novel thermostable cas12a enzyme (SLK-9) can achieve PCR-like high sensitivity and specificity detecting SARS-CoV-2 RNA from clinical samples. The workflow is simple, rapid, high-throughput and automation compatible. The two assays have the potential to reduce current COVID-19 diagnostic assay turnaround time and improve the throughput to all laboratories increasing their testing capacity without sacrificing performance.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

LENGTHY TABLES
The patent application contains a lengthy table section. A copy of the table is available in electronic form from the USPTO web site (https://seqdata.uspto.gov/?pageRequest=docDetail&DocID=US20240052436A1). An electronic copy of the table will also be available from the USPTO upon request and payment of the fee set forth in 37 CFR 1.19(b)(3).

1. A composition comprising
(i) a guide polynucleotide having a nucleotide sequence comprising a crRNA selected from the group consisting of: SEQ ID NO.: 77; SEQ ID NO.: 84; SEQ ID NO.: 91; and
(ii) a CRISPR/Cas enzyme.

2. The composition of claim 1, wherein the CRISPR/Cas enzyme is a Type V CRISPR/Cas enzyme.

3. The composition of claim 1, wherein the CRISPR/Cas enzyme is a Type VI CRISPR/Cas enzyme.

4. The composition of claim 1, wherein the CRISPR/Cas enzyme is a thermostable CRISPR/Cas enzyme.

5. The composition of claim 1, wherein the CRISPR/Cas enzyme is a Cas13 CRISPR/Cas enzyme.

6. A composition comprising:
a reverse transcriptase;
a DNA polymerase;
nucleotides;
RNA;
a primer set selected from the group consisting of:
(a):
orf1ab-F3
(SEQ ID NO. 72)
TGAAAATAGGACCTGAGCG
orf1ab-B3
(SEQ ID NO. 73)
ACACCTAGTCATGATTGCA
orf1ab-FIP
(SEQ ID NO. 74)
CCAATAGAATGATGCCAACAGGCGATAGACGTGCCACATGC
orf1ab-BIP
(SEQ ID NO. 75)
GATTGATGTTCAACAATGGGGTTTCATTACCATGGACTTGACAAT
orf1ab-LF-T7
(SEQ ID NO. 76)
gaaatTAATACGACTCACTATAGGGAAGTGTCTGAAGCAGTGGAAAA;
(b):
N-5F3
(SEQ ID NO. 78)
GCTTCTACGCAGAAGGGA
N-5B3
(SEQ ID NO. 79)
GTGACAGTTTGGCCTTGT
N-5FIP
(SEQ ID NO. 80)
TACTGCTGCCTGGAGTTGAATTCCTCTTCTCGTTCCTCATC
N-5BIP
(SEQ ID NO. 81)
GCTTTGCTGCTGCTTGACAGTGTTGTTGGCCTTTACCA
N-5LB
(SEQ ID NO. 82)
ATTGAACCAGCTTGAGAGCAAA
N-5LF-T7
(SEQ ID NO. 83)
gaaatTAATACGACTCACTATAGGGCTTGAACTGTTGCGACTACGT;
(c)
RP-F3
(SEQ ID NO. 85)
TTGATGAGCTGGAGCCA
RP-B3
(SEQ ID NO. 86)
CACCCTCAATGCAGAGTC
RP-FIP
(SEQ ID NO. 87)
GTGTGACCCTGAAGACTCGGTTTTAGCCACTGACTCGGATC
RP-BIP
(SEQ ID NO. 88)
CCTCCGTGATATGGCTCTTCGTTTTTTTCTTACATGGCTCTGGTC
RP-LF
(SEQ ID NO. 89)
ATGTGGATGGCTGAGTTGTT
RP-LB-T7
(SEQ ID NO. 90)
GAATTAATACGACTCACTATAGGGCATGCTGAGTACTGGACCTC;
and combinations thereof.

7. The composition of claim 6, wherein the RNA is isolated from a sample from a subject.

8. The composition of claim 7, wherein the subject is or is suspected to be or have been exposed to or infected with SARS-CoV-2.

9.-18. (canceled)

19. A method of detecting a SARS-CoV-2 target nucleic acid sequence, the method comprising:
contacting an RNA preparation with a CRISPR/Cas enzyme bound to a guide polynucleotide having a nucleotide sequence comprising a crRNA selected from the group consisting of: SEQ ID NO.: 77; SEQ ID NO.: 84; SEQ ID NO.: 91;
in the presence of rNTPs and a labeled nucleic acid reporter construct;
wherein cleavage of the labeled nucleic acid reporter construct by the CRISPR/Cas enzyme results in a detectable signal;
wherein detection of the detectable signal indicates presence of the SARS-CoV-2 target nucleic acid sequence in the RNA preparation.

20. A method of detecting a SARS-CoV-2 target nucleic acid sequence, the method comprising:
(i) obtaining a sample from a subject
(ii) isolating nucleic acid from the sample
(iii) amplifying target sequences by contacting the isolated nucleic acid with a primer set selected from the group consisting of:
(a):
orf1ab-F3
(SEQ ID NO. 72)
TGAAAATAGGACCTGAGCG
orf1ab-B3
(SEQ ID NO. 73)
ACACCTAGTCATGATTGCA
orf1ab-FIP
(SEQ ID NO. 74)
CCAATAGAATGATGCCAACAGGCGATAGACGTGCCACATGC
orf1ab-BIP
(SEQ ID NO. 75)
GATTGATGTTCAACAATGGGGTTTCATTACCATGGACTTGACAAT
orf1ab-LF-T7
(SEQ ID NO. 76)
gaaatTAATACGACTCACTATAGGGAAGTGTCTGAAGCAGTGGAAAA;
(b):
N-5F3
(SEQ ID NO. 78)
GCTTCTACGCAGAAGGGA
N-5B3
(SEQ ID NO. 79)
GTGACAGTTTGGCCTTGT
N-5FIP
(SEQ ID NO. 80)
TACTGCTGCCTGGAGTTGAATTCCTCTTCTCGTTCCTCATC
N-5BIP
(SEQ ID NO. 81)
GCTTTGCTGCTGCTTGACAGTGTTGTTGGCCTTTACCA
N-5LB
(SEQ ID NO. 82)
ATTGAACCAGCTTGAGAGCAAA
N-5LF-T7
(SEQ ID NO. 83)
gaaatTAATACGACTCACTATAGGGCTTGAACTGTTGCGACTACGT;
(c)
RP-F3
(SEQ ID NO. 85)
TTGATGAGCTGGAGCCA
RP-B3
(SEQ ID NO. 86)
CACCCTCAATGCAGAGTC
RP-FIP
(SEQ ID NO. 87)
GTGTGACCCTGAAGACTCGGTTTTAGCCACTGACTCGGATC
RP-BIP
(SEQ ID NO. 88)
CCTCCGTGATATGGCTCTTCGTTTTTTTCTTACATGGCTCTGGTC
RP-LF
(SEQ ID NO. 89)
ATGTGGATGGCTGAGTTGTT
RP-LB-T7
(SEQ ID NO. 90)
GAATTAATACGACTCACTATAGGGCATGCTGAGTACTGGACCTC;
and combinations thereof.
a reverse transcriptase;
a DNA polymerase;
nucleotides
(iv) contacting the amplified target sequences with:
a CRISPR/Cas enzyme;
a guide polynucleotide having a nucleotide sequence comprising a crRNA selected from the group consisting of: SEQ ID NO.: 77; SEQ ID NO.: 84; SEQ ID NO.: 91
rNTPs;
a labeled nucleic acid reporter construct;
wherein cleavage of the labeled nucleic acid reporter construct by the CRISPR/Cas enzyme results in a detectable signal;
wherein detection of the detectable signal indicates presence of the SARS-CoV-2 target nucleic acid sequence.

21. (canceled)

22. The method of claim 20, wherein the sample is a biological sample.

23. The method of claim 20, wherein the sample comprises nasal swab, nasopharyngeal swab, oropharyngeal swab, nasal aspirate, sputum, bronchoalveolar lavage, blood, serum, feces, and saliva.

24. The method of claim 20, wherein the step of isolating nucleic acid from the sample comprises isolating RNA from the sample.

25. The method of claim 20, wherein the labeled nucleic acid reporter construct is an RNA.

26. The method of claim 20, wherein the labeled nucleic acid reporter construct comprises a fluor/quencher pair.

27. The method of claim 20, wherein the detectable signal is fluorescence detection.

28. The composition of claim 20, wherein the CRISPR/Cas enzyme is a Type V CRISPR/Cas enzyme.

29. The composition of claim 20, wherein the CRISPR/Cas enzyme is a Type VI CRISPR/Cas enzyme.

30. The composition of claim 20, wherein the CRISPR/Cas enzyme is a thermostable CRISPR/Cas enzyme.

31. The composition of claim 20, wherein the CRISPR/Cas enzyme is a Cas13 CRISPR/Cas enzyme.

32. The composition of claim 20, wherein the CRISPR/Cas enzyme exhibits collateral RNase activity.

33.-41. (canceled)

42. A method of detecting a SARS-CoV-2 target nucleic acid sequence in a sample comprising:
contacting nucleic acid from the sample with:
a primer set having at least one primer selected from the group consisting of SEQ ID NOs.: 72-76, 78-83 92-429, 528-529 and JpS1-FIP AGGGACATAAGTCACATGCAAGAAGCTATCATCTTATGTCCTTCCCTC;
a type VI Cas;
at least one guide polynucleotide comprising a crRNA capable of binding the target nucleic acid sequence; and
a labeled nucleic acid reported construct, wherein the type VI Cas exhibits collateral RNase activity and cleaves the labeled nucleic acid reported construct once activated by presence of the target sequence;
detecting a signal from cleavage of labeled nucleic acid reported construct, thereby detecting the SARS-CoV-2 target nucleic acid sequence in the sample.

43. The method of claim 42, wherein the type VI Cas is Cas13.

44. The method of claim 42, further comprising obtaining a biological sample from a subject.

45. The method of claim 42, wherein the biological sample comprises nasal swab, nasopharyngeal swab, oropharyngeal swab, nasal aspirate, sputum, bronchoalveolar lavage, blood, feces, and saliva samples.

46. The method of claim 42, wherein the step of detecting a signal comprises detection of fluorescence, absorbance, spectrometry, lateral flow, migration, chemiluminescence, migration, electrochemical detection.

47.-60. (canceled)
